SEQUENCE LISTING (20 sequences)

SEQ ID NO: 1 - protein, 42 aa, Homo sapiens
  MOD_RES (42)..(42): Gln is modified with an OH group
YAEGTFISDYSIAMDKIHQQDFVNWLLAQKGKKNDWKHNITQ

SEQ ID NO: 2 - protein, 30 aa, Homo sapiens
  MOD_RES (30)..(30): Arg is modified with an NH2 group
HAEGTFTSDVSSYLEGQAAKEFIAWLVKGR

SEQ ID NO: 3 - protein, 39 aa, Heloderma suspectum
  MOD_RES (39)..(39): Ser is modified with an NH2 group
HGEGTFTSDLSKQMEEEAVRLFIEWLKNGGPSSGAPPPS

SEQ ID NO: 4 - protein, 39 aa, Artificial Sequence
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4- (17-carboxyheptadecanoylamino)butanoyl]amino]ethoxy]ethoxy] acetyl]amino]ethoxy]ethoxy]acetyl-Lys
  MOD_RES (20)..(20): Xaa is a 2-Methylalanine
  MOD_RES (39)..(39): Ser is modified with an NH2 group
YXEGTFISDLSIXKDRIHQXEFIEWLLAQGPSSGAPPPS

SEQ ID NO: 5 - protein, 39 aa, Artificial Sequence; identical to SEQ ID NO: 4
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[[2-[[2-[[(4S)-4-carboxy-4-(17-carboxyheptadecanoyl- amino)butanoyl]amino]acetyl]amino]acetyl]amino]acetyl]-Lys
  MOD_RES (20)..(20): Xaa is a 2-Methylalanine
  MOD_RES (39)..(39): Ser is modified with an NH2 group

SEQ ID NO: 6 - protein, 39 aa, Artificial Sequence; identical to SEQ ID NO: 4
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[methyl-[2-[methyl-[2-[methyl-[(4S)-4-carboxy-4- (17-carboxy-heptadecanoylamino)butanoyl]amino]acetyl]amino] acetyl]amino]acetyl]-Lys
  MOD_RES (20)..(20): Xaa is a 2-Methylalanine
  MOD_RES (39)..(39): Ser is modified with an NH2 group

SEQ ID NO: 7 - protein, 39 aa, Artificial Sequence; identical to SEQ ID NO: 4
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-(19- carboxynonadecanoylamino)butanoyl]amino]ethoxy]ethoxy]acetyl] amino]ethoxy]ethoxy]acetyl-Lys
  MOD_RES (20)..(20): Xaa is a 2-Methylalanine
  MOD_RES (39)..(39): Ser is modified with an NH2 group

SEQ ID NO: 8 - protein, 39 aa, Artificial Sequence; identical to SEQ ID NO: 4
  Exendin-4-analogue
  MOD_RES (1)..(1): Tyr is is functionalized at the N-terminus as N-Methyl-Tyr
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-(17- carboxyheptadecanoylamino)butanoyl]amino]ethoxy]ethoxy] acetyl]amino]ethoxy]ethoxy]acetyl-Lys
  MOD_RES (20)..(20): Xaa is a 2-Methylalanine
  MOD_RES (39)..(39): Ser is modified with an NH2 group

SEQ ID NO: 9 - protein, 39 aa, Artificial Sequence
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-(17- carboxyheptadecanoylamino)butanoyl]amino]ethoxy]ethoxy] acetyl]amino]ethoxy]ethoxy]acetyl-Lys
  MOD_RES (39)..(39): Ser is modified with an NH2 group
YXEGTFISDLSIXKDRIHQEEFIEWLLAQGPSSGAPPPS

SEQ ID NO: 10 - protein, 39 aa, Artificial Sequence; identical to SEQ ID NO: 9
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-(19- carboxynonadecanoylamino)butanoyl]amino]ethoxy] ethoxy]acetyl]amino]ethoxy]ethoxy]acetyl-Lys
  MOD_RES (39)..(39): Ser is modified with an NH2 group

SEQ ID NO: 11 - protein, 39 aa, Artificial Sequence; identical to SEQ ID NO: 9
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-(17- carboxyheptadecanoylamino)butanoyl]amino] ethoxy]ethoxy]acetyl]amino]ethoxy]ethoxy]acetyl-Lys
  MOD_RES (39)..(39): Ser is modified with an OH group

SEQ ID NO: 12 - protein, 39 aa, Artificial Sequence; identical to SEQ ID NO: 9
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[2-[2-[2-[(4S)-4-carboxy-4-(17- carboxyheptadecanoylamino)butanoyl]amino]ethoxy]ethoxy]acetyl] amino]ethoxy]ethoxy]acetyl]amino]ethoxy]ethoxy]acetyl-Lys
  MOD_RES (39)..(39): Ser is modified with an NH2 group

SEQ ID NO: 13 - protein, 39 aa, Artificial Sequence; identical to SEQ ID NO: 9
  Exendin-4-analogue
  MOD_RES (1)..(1): Tyr is functionalized at the N-terminus as N-Methyl-Tyr
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-(17- carboxyheptadecanoylamino)butanoyl]amino]ethoxy] ethoxy]acetyl]amino]ethoxy]ethoxy]acetyl-Lys
  MOD_RES (39)..(39): Ser is modified with an NH2 group

SEQ ID NO: 14 - protein, 39 aa, Artificial Sequence; identical to SEQ ID NO: 9
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-[[(4S)-4-carboxy-4- (17-carboxyheptadecanoylamino)butanoyl]amino]butanoyl]amino] ethoxy]-ethoxy]acetyl]amino]ethoxy]ethoxy]acetyl-Lys
  MOD_RES (39)..(39): Ser is modified with an NH2 group

SEQ ID NO: 15 - protein, 39 aa, Artificial Sequence; identical to SEQ ID NO: 9
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-[[(4S)-4-carboxy-4- (19-carboxynonadecanoylamino)butanoyl]amino]butanoyl]amino] ethoxy]-ethoxy]acetyl]amino]ethoxy]ethoxy]acetyl-Lys
  MOD_RES (39)..(39): Ser is modified with an NH2 group

SEQ ID NO: 16 - protein, 39 aa, Artificial Sequence; identical to SEQ ID NO: 9
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[[2-[[2-[[(4S)-4-carboxy-4-(17-carboxyheptadecanoyl- amino)butanoyl]amino]acetyl]amino]acetyl]amino]acetyl]-Lys
  MOD_RES (39)..(39): Ser is modified with an NH2 group

SEQ ID NO: 17 - protein, 39 aa, Artificial Sequence; identical to SEQ ID NO: 9
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[methyl-[2-[methyl-[2-[methyl-[(4S)-4-carboxy-4-(17- carboxy-heptadecanoylamino)butanoyl]amino]acetyl]amino] acetyl]amino]acetyl]-Lys
  MOD_RES (39)..(39): Ser is modified with an NH2 group

SEQ ID NO: 18 - protein, 39 aa, Artificial Sequence; identical to SEQ ID NO: 4
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-[[(4S)-4-carboxy-4- (17-carboxyheptadecanoylamino)butanoyl]amino]butanoyl]amino] ethoxy]-ethoxy]acetyl]amino]ethoxy]ethoxy]acetyl-Lys
  MOD_RES (20)..(20): Xaa is a 2-Methylalanine
  MOD_RES (39)..(39): Ser is modified with an NH2 group

SEQ ID NO: 19 - protein, 39 aa, Artificial Sequence; identical to SEQ ID NO: 4
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-[[(4S)-4-carboxy-4- (19-carboxynonadecanoylamino)butanoyl]amino]butanoyl]amino] ethoxy]-ethoxy]acetyl]amino]ethoxy]ethoxy]acetyl-Lys
  MOD_RES (20)..(20): Xaa is a 2-Methylalanine
  MOD_RES (39)..(39): Ser is modified with an NH2 group

SEQ ID NO: 20 - protein, 39 aa, Artificial Sequence; identical to SEQ ID NO: 4
  Exendin-4-analogue
  MOD_RES (2)..(2): Xaa is a 2-Methylalanine
  MOD_RES (13)..(13): Xaa is a 2-Methylalanine
  MOD_RES (14)..(14): Lys is functionalized at the amino side chain group as [2-[2-[2-[[2-[2-[2-[[(4S)-4-carboxy-4-(17- carboxyheptadecanoylamino)butanoyl]amino]ethoxy]ethoxy] acetyl]amino]ethoxy]ethoxy]acetyl-Lys
  MOD_RES (20)..(20): Xaa is a 2-Methylalanine
  MOD_RES (39)..(39): Ser is modified with an OH group

(19) United States
(12) Patent Application Publication
(10) Pub. No.: US 2023/0103631 A1
(43) Pub. Date: Apr. 6, 2023

(54) PEPTIDES AS SELECTIVE GIP RECEPTOR AGONISTS

(71) Applicant: Sanofi, Paris (FR)

(72) Inventors: Thomas BOEHME, Frankfurt am Main (DE); Andreas EVERS, Frankfurt am Main (DE); Dirk GRETZKE, Frankfurt am Main (DE); Tim KLOECKENER, Frankfurt am Main (DE); Anish KONKAR, Frankfurt am Main (DE); Ziyu LI, Frankfurt am Main (DE); Katrin LORENZ, Frankfurt am Main (DE); Stefania PFEIFFER-MAREK, Frankfurt am Main (DE); Michael WAGNER, Frankfurt am Main (DE)

(73) Assignee: Sanofi, Paris (FR)

(21) Appl. No.: 17/909,689

(22) PCT Filed: Mar. 4, 2021
(86) PCT No.: PCT/EP2021/055430

§ 371 Date: Sep. 6, 2022

(30) Foreign Application Priority Data
Mar. 6, 2020 (EP) 20305238.6

(51) Int. Cl. C07K 14/575; A61P 3/10

(52) U.S. Cl. CPC C07K 14/575; A61P 3/10

(57) ABSTRACT

New peptides as selective GIP receptor agonists The present invention relates to peptidic selective GIP receptor agonists and their medical use, for example in the treatment of disorders of the metabolic syndrome, including diabetes and obesity, hyperglycemia, as well as the treatment of disorders associated with nausea and vomiting.

### FIELD OF THE INVENTION

The present invention relates to new peptidic compounds which are selective GIP receptor agonists and their medical use, for example in the treatment of disorders of the metabolic syndrome, including diabetes and obesity, hyperglycemia, as well as the treatment of disorders associated with nausea and vomiting. The compounds of the invention are structurally derived from exendin-4 and show high solubility and stability at physiological conditions also in the presence of antimicrobial preservatives like m-cresol or phenol which makes them especially suited for combinations with other antidiabetic compounds. The exendin-4 peptide analogues show high in vitro potency at the GIP receptor with excellent selectivity towards other GPCRs, favourable physico-chemical properties, improved pharmacokinetic properties and beneficial in vivo effects in relevant animal models.

### BACKGROUND OF THE INVENTION

GIP and GLP-1 are the two gut enteroendocrine cell-derived hormones accounting for the incretin effect, which accounts for over 70% of the insulin response to an oral glucose challenge (Baggio et al., Gastroenterology 2007, 132, 2131).

GIP (glucose-dependent insulinotropic polypeptide), also referred to as hGIP or hGIP(1-42), is a 42 amino acid peptide that is released from intestinal K-cells following food intake.

GIP's amino acid sequence is shown as SEQ ID NO: 1.

H₂N-YAEGTFISDYSIAMDKIHQQDFVNWLLAQKGKKNDWKHNITQ-OH

GIP and its analogs produce glucose-dependent insulin secretion from beta-cells thus exerting glucose control without risk for hypoglycemia. GIP exhibits glucoregulatory effects as a result of its direct effect on pancreatic islets (Taminato et al., Diabetes 1977, 26, 480; Adrian et al., Diabetologia 1978, 14, 413; Lupi et al., Regul Pept 2010, 165, 129). In addition, GIP analogs produce glucagon secretion from alpha cells in normal and diabetic humans (Chia et al., Diabetes 2009, 58, 1342; Christensen et al., Diabetes 2011, 60, 3103). This effect has the potential to further minimize hypoglycemic risk in diabetic subjects that lack hypoglycemia awareness. GIP peptides have also been shown to produce beneficial effect on bone and neuroprotection in preclinical models, effects if translated to humans may be of value in older diabetic subjects (Ding et al., J Bone Miner Res 2008, 23, 536; Verma et al., Expert Opin Ther Targets 2018, 22, 615; Christensen et al., J Clin Endocrinol Metab 2018, 103, 288). In addition, preclinical data indicates that GIP may have an anti-emetic effect and prevent emesis elicited by mechanisms (e.g. PYY) that induce nausea and vomiting in preclinical animal models (US 2018/0298070).

GLP-1 (Glucagon-like peptide 1) is a 30 amino acid peptide produced in intestinal epithelial endocrine L-cells.

The amino acid sequence of GLP-1(7-36)-amide is shown as SEQ ID NO: 2.

H₂N-HAEGTFTSDVSSYLEGQAAKEFIAWLVKGR-NH₂

Holst (Physiol. Rev. 2007, 87, 1409) and Meier (Nat. Rev. Endocrinol. 2012, 8, 728) show that GLP-1 receptor agonists improve glycemic control in patients with type 2 diabetes mellitus (T2DM) by reducing fasting and postprandial glucose (FPG and PPG) levels.

Exendin-4 (SEQ ID NO: 3) is a 39 amino acid peptide which is produced by the salivary glands of the Gila monster (Heloderma suspectum). Exendin-4 is an activator of the GLP-1 receptor, whereas it shows only very low activation of the GIP receptor and does not activate the glucagon receptor (Finan et al., Sci. Transl. Med. 2013, 5(209). 151).

The amino acid sequence of exendin-4 is shown as SEQ ID NO: 3.

H₂N-HGEGTFTSDLSKQMEEEAVRLFIEWLKNGGPSSGAPPPS-NH₂

Exendin-4 shares many of the glucoregulatory actions observed with GLP-1 (GLP-1(7-36) amide: SEQ ID NO: 2). Clinical and non-clinical studies have shown that exendin-4 has several beneficial antidiabetic properties including a glucose-dependent enhancement in insulin synthesis and secretion, glucose-dependent suppression of glucagon secretion, slowing down gastric emptying, reduction of food intake and body weight, and an increase in beta-cell mass and markers of beta-cell function.

These effects may be beneficial not only for diabetics but also for patients suffering from obesity. Patients with obesity have a higher risk of getting diabetes, hypertension, hyperlipidemia, cardiovascular and musculoskeletal diseases.

Compared to GLP-1, glucagon and oxyntomodulin, exendin-4 has beneficial physicochemical properties, such as solubility and stability in solution and under physiological conditions (including enzymatic stability towards degradation by enzymes, such as DPP4 or NEP), which results in a longer duration of action in vivo.

Nevertheless, also exendin-4 has been shown to be chemically labile due to methionine oxidation in position 14 (Hargrove et al., Regul. Pept. 2007, 141, 113) as well as deamidation and isomerization of asparagine in position 28 (WO 2004/035623 A2). Therefore, stability might be improved by substitution of methionine at position 14 and the avoidance of sequences that are known to be prone to degradation via aspartimide formation, especially Asp-Gly or Asn-Gly at positions 28 and 29.

Co-Activation of GLP-1 and GIP Receptors

It has been described that dual activation of the GLP-1 and GIP receptors, e.g. by combining the actions of GLP-1 and GIP in one preparation, leads to a therapeutic principle with significantly better reduction of blood glucose levels, increased insulin secretion and reduced body weight in mice with T2DM and obesity compared to the marketed GLP-1 agonist liraglutide (e.g. Gault et al., Clin Sci (Lond) 2011, 121, 107), an effect if translated to humans may be of value for treatment of obesity or metabolic disorders. Native GLP-1 and GIP were proven in humans following co-infusion to interact in an additive manner with a significantly increased insulinotropic effect compared to GLP-1 alone (Nauck et al., J. Clin. Endocrinol. Metab. 1993, 76, 912).

Finan et al. (Sci. Transl. Med. 2013, 5, 151), Frias et al. (Cell Metab. 2017, 26, 343), Portron et al (Diabetes Obes. Metab. 2017, 19, 1446) as well as Coskun et al. (Mol. Metab. 2018, 18, 3) describe dual agonists of the GLP-1 and the GIP receptors by combining the actions of GLP-1 and GIP in one molecule. This leads to a therapeutic principle with anti-diabetic action, body weight loss and a pronounced glucose lowering effect superior to pure GLP-1 agonists, among others due to GIP receptor mediated increase in insulin secretion.

Dual peptidic agonists of the GLP-1 receptor and the GIP receptor designed as analogues of exendin-4 and substituted with a fatty acid side chain are described in patent applications WO 2014/096145 A1, WO 2014/096150 A1, WO 2014/096149 A1, and WO 2014/096148 A1, as well as in patent applications WO 2011/119657 A1, WO 2016/111971 A1, WO 2016/131893 A1 and WO2020/023386 A1. GLP-1 and GIP receptor agonists based on exendin-4 and stabilized by non-genetically encoded amino acids are also described in patent applications WO 2015/086730 A1, WO 2015/086729 A1, and WO 2015/086728 A1.

Specific GIP Receptor Agonists

GIP receptor agonists when co-administered with GLP-1 analogs enhance the efficacy of selective GLP-1R agonists on glycemic control and body weight loss in preclinical models. To be able to identify the ideal ratio of GIP receptor and GLP-1 receptor activation, e.g. to achieve maximum effects on body weight loss in patients, and to treat patients with the ideal doses of the respective GPCR receptor agonists, compounds that selectively activate the GIP receptor are needed,

Attempts have been made to find peptides with high affinity for the GIP receptor and high agonistic activity at the GIP receptor but diminished affinity for the GLP-1 receptor, that also have favourable physicochemical properties and an extended half-life. GIP itself is prone to aggregation and fibrillation in aqueous solution and has a very short half-life of 2 min (Meier et al., Diabetes 2004, 53, 654).

Specific GIP receptor agonists stabilized by non-genetically encoded amino acids and/or lipid side chain substitution are described in Tatarkiewicz et al., Diabetes Obes. Metab. 2014, 16, 75. GIP receptor agonists with protracted activity profile via specific lipid side chain substitution and their use as therapeutic agents are described in WO 2012/055770, and WO 2018/181864, GIP receptor agonists based on the natural human GIP sequence are disclosed in patent applications, such as e.g. WO 2019/211451. GIP receptor agonists based on the exendin-4 sequence and their potential medical use are disclosed in Piotr A. Mroz et al., Molecular Metabolism, vol. 20, 2018, 51-62 and in patent applications, such as WO 2016/066744 A2.

However, a high selectivity in binding to the GIP receptor as opposed to the GLP-1 receptor has not been disclosed to have been accomplished in these peptides. To achieve a very high activation of the GIP receptor, high doses of the GIP peptide have to be administered. At high doses, an antagonistic effect of these peptides on the GLP-1 receptor cannot be excluded if the binding affinity towards GLP-1 receptor is not diminished.

Thus, there still is a need for highly selective highly GIP receptor-selective peptide agonists which that are highly soluble, stable in solution and have a long in vivo half-life.

The inventors have surprisingly found that peptides of the invention have a high binding selectivity for the GIP receptor as compared to the GLP-1 receptor, selectively activate the GIP receptor and have good physicochemical properties, such as being highly soluble, and chemically as well as physically stable in aqueous solutions in the absence and presence of antimicrobial preservatives like m-cresol or phenol. Further, the peptides of the invention have glucose-lowering activity and a prolonged half-life in vivo.

In a first aspect, peptides of the invention bind to the GIP receptor with high affinity. In a further aspect, the peptides of the invention are selective in binding to the GIP receptor over the GLP-1 receptor with an at least 100-fold split.

In a further aspect, peptides of the invention are activating the GIP receptor. In a further aspect, peptides of the invention are activating the GIP receptor over the GLP-1 receptor with an at least 1000-fold split.

Also, the peptides of the invention have an improved pharmacokinetic profile in vivo.

Also, peptides of the invention have an improved physical and/or chemical stability in aqueous solutions.

Also, peptides of the invention are active in vivo alone or in combination with a GLP-1 receptor agonist.

### DESCRIPTION OF THE INVENTION

A problem associated with the use of peptidic compounds as a therapeutic in the treatment of diabetes, obesity, metabolic syndrome and other indications is their limited half-life in vivo. Therefore, peptidic sequences are stabilized by introduction of non-genetically encoded amino acids to enhance stability against proteases and/or substituted with fatty acid side chains to allow interaction with plasma proteins as albumin to prolong the residence time in plasma and/or administered in depot formulations to allow sustained levels of active compound in the circulation.

Therefore, in developing new therapeutic molecules, there is a need for variants with improved pharmaceutical properties, in particular increased stability against proteases and/or increased chemical or physical stability and/or a prolonged half-life in vivo and/or increased potency/efficacy in vivo.

There is also a need for additional glucose lowering therapies, particularly with therapeutics that show beneficial physico-chemical properties also in the presence of phenolic preservatives.

Also, there remains a need for glucose lowering therapies that avoid or even alleviate the common gastrointestinal side effects of GLP-1 based therapies (namely nausea and vomiting), thereby achieving a strong glucose lowering effect with improved tolerability.

The prior art cited above discloses peptidic agonists of the GIP receptor for formulation at physiological pH. The present inventors surprisingly found that compounds of this invention show favorable physico-chemical properties, also in the presence of phenolic preservatives, e.g. high solubility as well as good chemical and physical stability, combined with high activity on the GIP receptor, high selectivity versus the GLP-1 receptor, prolonged half-life and good in vivo activity.

Native exendin-4 is a pure GLP-1 receptor agonist without activity on the glucagon receptor and very low activity on the GIP receptor. The compounds of the invention are based on the structure of native exendin-4, but are different at sixteen positions compared to SEQ ID NO: 3. These differences contribute to the enhancement of the agonistic activity at the GIP receptor, diminish affinity towards the GLP-1 receptor and eliminate the agonistic activity at the GLP-1 receptor.

Characteristic structural motifs of the compounds of the invention are: Tyr at position 1, Aib at position 2, Ile at position 7, Leu at position 10, Ile at position 12, Aib at position 13, Asp at position 15, Arg at position 16, Ile at position 17, His at position 18, Gln at position 19, Glu or Aib at position 20, Leu at position 27, Ala at position 28 and Gln at position 29.

Among other substitutions, methionine at position 14 is replaced by an amino acid carrying an —NH₂ group in the sidechain, which is further substituted by a lipophilic residue (e.g. a fatty acid combined with a linker). The additional replacement of the exendin-4 amino acids at positions 1, 2, 7, 12, 13, 15, 16, 17, 18, 19, 20, 21, as well 25 as positions 27, 28 and 29 with Tyr at position 1, Aib at position 2, Ile at position 7, Ile at position 12, Aib at position 13, Asp at position 15, Arg at position 16, Ile at position 17, His at position 18, Gln at position 19, Glu or Aib at position 20, as well as Leu at position 27, Ala at position 28 and Gln at position 29 provides peptides with high activity at the GIP receptor, without agonistic activity at the GLP-1 receptor.

Commonly, high GIP receptor agonistic activity is instilled into a peptidic entity by incorporating consecutive stretches of the natural human GIP hormone (SEQ ID NO: 1), e.g. Tyr-Ser-Ile-Ala at positions 10 to 13 and Lys-Ile-His-Gln at positions 16 to 19. This potentially results in peptides with poor physical stability properties leading to fibrillation in a ThT binding assay (as described in Methods). Surprisingly, it was found that peptides of the present invention that do not contain amino acids from the natural GIP hormone in positions 10, 13, 16, 20 and 21 are peptides with very high GIP receptor agonism and favorable solubility as well as chemical and physical stability also in the presence of phenolic preservatives as shown in the respective examples.

Therefore, the present invention provides novel exendin-4 derived peptides having solely GIP receptor agonist activity. The peptides of this invention show high chemical stability, solubility and physical stability at physiological pH values, such as pH 7.4, also in the presence of phenolic antimicrobial preservatives. Further provided are medical uses of the claimed peptides.

The invention relates to compounds of the formula I

I
R¹HN-Tyr-Aib-Glu-Gly-Thr-Phe-Ile-Ser-Asp-
Leu-Ser-Ile-Aib-X14-Asp-Arg-Ile-His
Gln-X20-Glu-Phe-Ile-Glu-Trp-Leu-Leu-Ala-
Gln-Gly-Pro-Ser-Ser-Gly-Ala-Pro-Pro-Pro-
Ser-R²

wherein
R¹ is H or C₁-C₄-alkyl
X14 represents Lys wherein the —NH₂ side chain group is functionalized by —Z1-Z2-C(O)—R⁵, wherein
   —Z1-Z2- represents a linker in all stereoisomeric forms and
   R⁵ is a moiety comprising up to 70 carbon atoms and heteroatoms selected from N and O,
X20 represents an amino acid residue selected from Glu and Aib,
R² is NH₂ or OH,
or a salt or solvate thereof.

The Lys residue with an —NH₂ side chain group, is functionalized in that at least one H atom of the —NH₂ side chain group is replaced by —Z1-Z2-C(O)—R⁵, wherein R⁵ comprises a lipophilic moiety, e.g. an acyclic linear or branched (C₈-C₃₀) saturated or unsaturated hydrocarbon group, which is unsubstituted or substituted e.g. by halogen (F, Cl, Br, J), —OH and/or —CO₂H and

Z1-Z2 comprises a linker in all stereoisomeric forms, e.g. a linker comprising one or more, e.g. 1 to 5, preferably 1, 2 or 3 amino acid linker groups selected from the group gamma-glutamate (gGlu), glycine (Gly), N-Methyl-glycine (N-MeGly) and 8-amino-3,6-dioxa-octanoic acid (AEEA).

Preferred groups R⁵ comprise a lipophilic moiety, e.g. an acyclic linear or branched (C₁₂-C₂₀) saturated or unsaturated hydrocarbon group, e.g. pentadecanyl, hexadecanyl, heptadecanyl or nonadecanyl, which is unsubstituted or substituted by CO₂H, more preferably 17-carboxy-heptadecanoyl, or 19-carboxynonadecanoyl. In one embodiment the amino acid linker group is AEEA-AEEA-gGlu. In another embodiment the amino acid linker group is AEEA-AEEA-AEEA-gGlu. In another embodiment the amino acid linker group is AEEA-AEEA-gGlu-gGlu. In another embodiment the amino acid linker group is Gly-Gly-Gly-gGlu. In another embodiment the amino acid linker group is (N-MeGly)-(N-MeGly)-(N-MeGly)-gGlu.

Compounds of the invention have GIP activity. This term refers to the ability to bind to the GIP receptor and initiate a signal transduction pathway resulting in insulinotropic action or other physiological effects as is known in the art. For example, compounds of the invention can be tested for GIP receptor affinity or activity using the assays described in Methods and results shown in Examples 10-12 herein.

The compounds of the invention are selective GIP receptor agonists as determined by the observation that they are capable of stimulating intracellular cAMP formation in the assay systems described in Methods (HEK cell agonism).

According to another embodiment the compounds of the invention, particularly with a lysine at position 14 which is further substituted with a lipophilic residue, exhibit at least an activity determined using the method of Example 10 without albumin of 10 pM at the GIP receptor (i.e. EC50≤10 pM), more preferably of 5 pM (i.e. EC50≤5 pM), more preferably of 1 pM (i.e. EC50≤1.0 pM) and even more preferably of 0.36 pM (i.e. EC50≤0.36 pM) in the respective assay system—HEK cell agonism as described in Example 10 without albumin.

Furthermore, the compounds of the invention are GIP receptor agonists as determined by the observation that they are capable of stimulating intracellular cAMP formation in human adipocytes in the assay system described in Methods.

According to another embodiment the compounds of the invention, particularly with a lysine at position 14 which is further substituted with a lipophilic residue, exhibit at least an activity determined using the method of Example 11 of 10 nM at the GIP receptor (i.e. EC50≤10 nM), more preferably of 8 nM (i.e. EC50≤8.0 nM), more preferably of 4.6 nM (i.e. EC50≤4.6 nM) and even more preferably of 2 nM (i.e. EC50≤2.0 nM) in the respective assay system—human adipocytes agonism as described in Example 11.

In a further aspect, the compounds of the invention are selective at activating the human GIP receptor over the human GLP-1 receptor.

According to another embodiment the compounds of the invention, particularly with a lysine at position 14 which is further substituted with a lipophilic residue, exhibit no or weak activity at the GLP-1 receptor with an EC50 as determined using the method of Example 10 without albumin of more than 100 pM (i.e. EC50>100 pM), more preferably of more than 1000 pM (i.e. EC50>1000 pM), more preferably of 5000 pM (i.e. EC50>5000 pM) and even more preferably of 10000 pM (i.e. EC50>10000 pM) in the respective assay system—HEK cell agonism as described in Example 10 without albumin.

The compounds of the invention bind to the GIP receptor as determined by the observation that they are capable of displacing [¹²⁵I]-GIP from the GIP receptor in the assay system described in Methods.

The compounds of the invention bind to the hGIP receptor as determined using the method of Example 12 with an IC50 of 10 nM or less (i.e. IC50≤10 nM), more preferably 8 nM or less (i.e. IC50≤8.0 nM), more preferably 5 nM or less (i.e. IC50≤5.0 nM), more preferably 3.13 nM or less (i.e. IC50≤3.13 nM) and even more preferably 1 nM or less (i.e. IC50≤1.0 nM).

Furthermore, the compounds of the invention bind only weakly to the GLP-1 receptor as determined by the observation that they are capable of displacing [¹²⁵I]GLP-1 from the GLP-1 receptor in the assay system described in Methods.

The compounds of the invention bind weakly to the hGLP-1 receptor as determined using the method of Example 12 with an IC50 of more than 10 nM (i.e. IC50>10 nM), more preferably more than 50 nM (i.e. IC50>50 nM), and even more preferably more than 100 nM (i.e. IC50>100 nM).

In a further aspect, the compounds of the invention are selective at binding to the human GIP receptor over the human GLP-1 receptor.

The term “activity” as used herein preferably refers to the capability of a compound to activate the human GIP receptor or the human GLP-1 receptor, particularly selectively the GIP receptor and not the GLP-1 receptor. More preferably the term “activity” as used herein refers to the capability of a compound to stimulate intracellular cAMP formation. The term “relative activity” as used herein is understood to refer to the capability of a compound to activate a receptor in a certain ratio as compared to another receptor agonist or as compared to another receptor. The activation of the receptors by the agonists (e.g. by measuring the cAMP level) is determined as described herein, e.g. as described in the examples. Sometimes, reference may also be made to the term “potency” or “in vitro potency” instead of “activity”. Accordingly, “potency” is a measure for the ability of a compound to activate the receptors for GLP-1 or GIP in a cell-based assay. Numerically, it is expressed as the “EC50 value” or “EC₅₀ value”, which is the effective concentration of a compound that induces a half-maximal increase of response (e.g. formation of intracellular cAMP) in a concentration-response experiment.

In a further particular embodiment, the derivatives of the invention are capable of activating the GIP receptor selectively over the human GLP-1 receptor. The term “selectively” when used in relation to activation of the GIP receptor over the GLP-1 receptor refers to derivatives that display at least 10-fold, such as at least 50-fold, at least 500-fold, or at least 1000-fold better potency for the GIP receptor over the GLP-1 receptor as measured in vitro in a potency assay for receptor function, such as described in Methods, and compared by EC50 values.

The compounds of the invention preferably have an EC50 for hGIP receptor determined using the method of Example 10 without albumin of 10 pM or less, preferably of 5 pM or less, more preferably of 1 pM or less, and even more preferably of 0.36 pM or less and an EC50 for hGLP-1 receptor of 100 pM or more, preferably of 1000 pM or more, more preferably of 5000 pM or more, and even more preferably of 10000 pM or more. The EC50 for the hGLP-1 receptor and the hGIP receptor may be determined as described in the Methods herein and are used to generate the results described in Example 10.

The compounds of formula I do show high activity at the GIP receptor but not at the GLP-1 receptor. The high activity at the GIP receptor is intended for enhanced efficacy on blood glucose control and body weight loss and to reduce the probability of GLP-1 related side effects like gastrointestinal distress.

The term “binding” as used herein preferably refers to the capability of a compound to bind to the human GIP receptor or the human GLP-1 receptor, particularly selectively to the GIP receptor. Sometimes, reference may also be made to the term “affinity” instead of “binding”. More preferably the term “binding” as used herein refers to the capability of a compound to displace a radioactively labelled compound from the respective receptor in the binding assay, e.g. [¹²⁵I]GIP from the GIP receptor as described in Methods and shown in Examples. Numerically, it is expressed as the “IC50 value”, which is the effective concentration of a compound that displaces half of the radioactively labelled compound from the receptor in a dose-response experiment.

The compounds of the invention preferably have an IC50 for hGIP receptor of 10 nM or less, preferably of 8 nM or less, more preferably of 5 nM or less, more preferably of 3.13 nM or less, and even more preferably of 1 nM or less and an IC50 for hGLP-1 receptor of 10 nM or more, preferably of 50 nM or more, and more preferably of 100 nM or more. The IC50 for the hGLP-1 receptor and the hGIP receptor may be determined as described in the Methods herein and as used to generate the results described in Example 12.

In one embodiment the compounds of the invention have a high solubility at physiological pH values, e.g. at a physiological range from pH 6 to 8, especially at pH 7.0 or pH 7.4 at 25° C., in another embodiment at least 1 mg/ml, in another embodiment at least 5 mg/ml and in a particular embodiment at least 10 mg/ml.

In one embodiment the compounds of the invention have a high solubility at physiological pH values in the presence of an antimicrobial preservative like phenol or m-cresol, e.g. at a physiological range from pH 6 to 8, especially at pH 7.0 or pH 7.4 at 25° C., in another embodiment at least 1 mg/ml, in another embodiment at least 5 mg/ml and in a particular embodiment at least 10 mg/ml.

Furthermore, the compounds of the invention preferably have a high chemical stability when stored in solution. Preferred assay conditions for determining the stability is storage for 28 days at 25° C. or 40° C. in solution at a physiological range from pH 7 to 8, especially pH 7.4. The stability of the compounds of the invention is determined by chromatographic analyses as described in the Methods. Preferably, after 28 days at 40° C. in solution at pH 7.4 the purity loss is no more than 15%, more preferably no more than 10% and even more preferably no more than 8%.

Furthermore, the compounds of the invention preferably have a high chemical stability when stored in solution in the presence of an antimicrobial preservative like phenol or m-cresol. Preferred assay conditions for determining the stability is storage for 28 days at 25° C. or 40° C. in solution at a physiological range from pH 7 to 8, especially pH 7.4. The stability of the compounds of the invention is determined by chromatographic analyses as described in the Methods. Preferably, after 28 days at 40° C. in solution at pH 7.4 the purity loss is no more than 15%, more preferably no more than 10% and even more preferably no more than 8%.

Furthermore, the compounds of the invention preferably have a high physical stability when stored in solution. Preferred assay conditions for determining the stability is storage for 28 days at 25° C. or 40° C. in solution at a physiological range from pH 7 to 8, especially pH 7.4.

In one embodiment the compounds of the invention do not show an increase in fluorescence intensity with Thioflavin T as fluorescence probe at concentrations of 3 mg/ml, e.g. at a physiological range from pH 7 to 8, especially pH 7.4, at 37° C. over 5 hours, more preferably over 10 h, more preferably over 20 h, more preferably over 30 h, more preferably over 40 h and even more preferably over 45 h as assayed by the ThT assay as described in Methods.

In one embodiment the compounds of the invention do not show an increase in fluorescence intensity with Thioflavin T as fluorescence probe at concentrations of 3 mg/ml in the presence of an antimicrobial preservative like phenol or m-cresol, e.g. at an acidity range from pH 7 to 8, especially pH 7.4, at 37° C. over 5 h, more preferably over 10 h, more preferably over 20 h, more preferably over 30 h, more preferably over 40 h and even more preferably over 45 h as assayed by the ThT assay as described in Methods.

In one embodiment the compounds of this invention are more resistant to cleavage by neutral endopeptidase (NEP) and dipeptidyl peptidase-4 (DPP4), resulting in a longer half-life and duration of action in vivo, when compared with native GIP or exendin-4.

In one embodiment the compounds of this invention are strongly bound to human albumin, resulting in a longer half-life and duration of action in vivo when compared with native human GIP.

The pharmacokinetic properties of the compounds of the invention may be determined in vivo in pharmacokinetic (PK) studies. Such studies are conducted to evaluate how pharmaceutical compounds are absorbed, distributed, and eliminated in the body, and how these processes affect the concentration of the compound in the body, over the course of time. In the discovery and preclinical phase of pharmaceutical drug development, animal models such as the mouse, rat, monkey, dog, or pig, may be used to perform this characterisation. Any of these models can be used to test the pharmacokinetic properties of the derivatives of the invention. In such studies, animals are typically administered a single dose of the drug, either intravenously (i.v.), or subcutaneously (s.c.) in a relevant formulation. Blood samples are drawn at predefined time points after dosing, and samples are analysed for concentration of drug with a relevant quantitative assay. Based on these measurements, time-plasma concentration profiles for the compound of study are plotted and a so-called non-compartmental pharmacokinetic analysis of the data is performed.

In one embodiment, the pharmacokinetic properties may be determined as terminal half-life (T_{1/2}) in vivo in minipigs after i. v. and s.c. administration, e. g. as described in Example 13 herein.

In particular embodiments, the terminal half-life in minipigs is at least 24 h, preferably at least 40 h, even more preferably at least 60 h.

In one embodiment, the pharmacokinetic properties may be determined as terminal half-life (T_{1/2}) in vivo in cynomolgous monkeys after i. v. and s.c. administration, e. g. as described in Example 13 herein.

In particular embodiments, the terminal half-life in monkeys is at least 24 h, preferably at least 40 h, even more preferably at least 50 h.

In one embodiment, the compounds of this invention are active in vivo alone or in combination with a GLP-1 receptor agonist.

The effect of compounds of the invention on glucose tolerance may be determined in mouse in vivo experiments by performing an oral or intraperitoneal (i.p) glucose tolerance test (oGTT or ipGTT), e. g. as described in Example 14 herein in C57Bl/6 mice. These tests are performed by administration of a glucose load orally or i. p. to semi-fasted animals and subsequent blood glucose measurement.

Mouse models can also be used to evaluate effects on body weight, food intake and glucose tolerance, e.g. DIO mice.

In one embodiment the compounds of the present invention comprise a peptide moiety which is a linear sequence of 39 amino carboxylic acids, particularly α-amino carboxylic acids linked by peptide, i.e. carboxamide bonds.

One embodiment of the invention are compounds of the formula I

I
R¹HN-Tyr-Aib-Glu-Gly-Thr-Phe-Ile-Ser-Asp-
Leu-Ser-Ile-Aib-X14-Asp-Arg-Ile-His
Gln-X20-Glu-Phe-Ile-Glu-Trp-Leu-Leu-Ala-
Gln-Gly-Pro-Ser-Ser-Gly-Ala-Pro-
Pro-Pro-Ser-R²1

wherein
R¹ is selected from H or methyl, ethyl, propyl, isopropyl, butyl, isobutyl or t-butyl,
X14 represents Lys wherein the —NH₂ side chain group is functionalized by —Z1-Z2-C(O)—R⁵, wherein
   —Z1-Z2- represents a linker in all stereoisomeric forms and
   R⁵ is a moiety comprising up to 70 carbon atoms and heteroatoms selected from N and O,
X20 represents an amino acid residue selected from Glu and Aib,
R² is NH₂ or OH,
or a salt or solvate thereof.

In one embodiment of the compound of formula I, R¹ is H or methyl.

In one embodiment of the compound of formula I, R¹ is H.

In one embodiment of the compound of formula I, R¹ is methyl.

In one embodiment of the compound of formula I, R² is NH₂ or OH.

In one embodiment of the compound of formula I, R² is NH₂.

In one embodiment of the compound of formula I, R² is OH.

A further embodiment relates to compounds of the formula I

I
R¹HN-Tyr-Aib-Glu-Gly-Thr-Phe-Ile-Ser-Asp-
Leu-Ser-Ile-Aib-X14-Asp-Arg-Ile-His
Gln-X20-Glu-Phe-Ile-Glu-Trp-Leu-Leu-Ala-
Gln-Gly-Pro-Ser-Ser-Gly-Ala-Pro-
Pro-Pro-Ser-R²

wherein
R¹ is H or C₁-C₄-alkyl,
X14 represents Lys wherein the —NH₂ side chain group is functionalized by —Z1-Z2-C(O)—R⁵, wherein
—Z1-Z2 represents a linker in all stereoisomeric forms comprising 1 to 5 amino acid linker groups selected from the group gamma-glutamate (gGlu), glycine (Gly), N-Methyl-glycine (N-MeGly) and 8-amino-3,6-dioxa-octanoic acid (AEEA), and.
R⁵ is an acyclic linear or branched (C₈-C₃₀) saturated or unsaturated hydrocarbon group, which is unsubstituted or substituted by halogen, —OH and/or —CO₂H,
X20 represents an amino acid residue selected from Glu and Aib,
R² is NH₂ or OH,
or a salt or solvate thereof.

A further embodiment relates to compounds of formula I, wherein
X14 represents Lys wherein the —NH₂ side chain group is functionalized with a group-Z1-Z2-C(O)R⁵, wherein
Z1 represents a group selected from AEEA, {AEEA}2, {AEEA}3, Gly, Gly-Gly, {Gly}3, N-MeGly, {N-MeGly}2, {N-MeGly}3;
Z2 represents a group selected from gGlu, or gGlu-gGlu; and
R⁵ represents a group —(CH2)x-COOH, wherein x is an integer from 15 to 22.

A further embodiment relates to compounds of formula I, wherein
X14 represents Lys wherein the —NH₂ side chain group is functionalized with a group-Z1-Z2-C(O)R⁵, wherein
Z1 represents a group selected from {AEEA}2, {AEEA}3, {Gly}3, {N-MeGly}3;
Z2 represents a group selected from gGlu, or gGlu-gGlu; and
R⁵ represents a group —(CH2)x-COOH, wherein x is an integer from 15 to 22.

A further embodiment relates to compounds of formula I, wherein
X14 represents Lys wherein the —NH₂ side chain group is functionalized with a group-Z1-Z2-C(O)R⁵, wherein
Z1 represents a group selected from AEEA, {AEEA}2, {AEEA}3, Gly, Gly-Gly, {Gly}3, N-MeGly, {N-MeGly}2, {N-MeGly}3;
Z2 represents a group selected from gGlu, or gGlu-gGlu; and
R⁵ represents 17-carboxy-1-oxoheptadecyl or 19-carboxy-1-oxononadecyl.

A further embodiment relates to compounds of formula I, wherein
X14 represents Lys wherein the —NH₂ side chain group is functionalized with a group-Z1-Z2-C(O)R⁵, wherein
Z1 represents a group selected from {AEEA}2, {AEEA}3, {Gly}3, {N-MeGly}3;
Z2 represents a group selected from gGlu, or gGlu-gGlu; and
R⁵ represents 17-carboxy-1-oxoheptadecyl or 19-carboxy-1-oxononadecyl.

Specific preferred examples for-Z1-Z2-C(O)—R⁵ groups are listed in the following Table 1, which are selected from
[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl-l N-(17-carboxy-1-oxoheptadecyl)-L-γ-glutamyl-2-[2-(2-aminoethoxy)ethoxy]acetyl-2-[2-(2-aminoethoxy)ethoxy]acetyl-,
[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl-l N-(19-carboxy-1-oxononadecyl)-L-γ-glutamyl-2-[2-(2-aminoethoxy)ethoxy]acetyl-2-[2-(2-aminoethoxy)ethoxy]acetyl-,
[2-[2-[2-[[2-[2-[2-[[2-[2-[2-[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl-,
[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]-ethoxy]acetyl]amino]ethoxy]ethoxy]acetyl-,
[2-[2-[2-[[2-[2-[2-[[(4S)-4-carboxy-4-[[(4S)-4-carboxy-4-(19-carboxynonadecanoylamino)butanoyl]amino]butanoyl]amino]ethoxy]-ethoxy]acetyl]amino]ethoxy]ethoxy]acetyl-,
[2-[[2-[[2-[[(4S)-4-carboxy-4-(17-carboxyheptadecanoyl-amino)butanoyl]amino]acetyl]amino]acetyl]amino]acetyl]-,
[2-[methyl-[2-[methyl-[2-[methyl-[(4S)-4-carboxy-4-(17-carboxy-heptadecanoylamino)butanoyl]amino]acetyl]amino]acetyl]amino]acetyl].

Further preferred are stereoisomers, particularly enantiomers of these groups, either S- or R-enantiomers. The term “R” in Table 1 is intended to mean the attachment site of —Z—C(O)—R⁵ at the peptide back bone, for example the epsilon-amino group of Lys.

TABLE 1

| Structure / IUPAC | name |
|---|---|
| 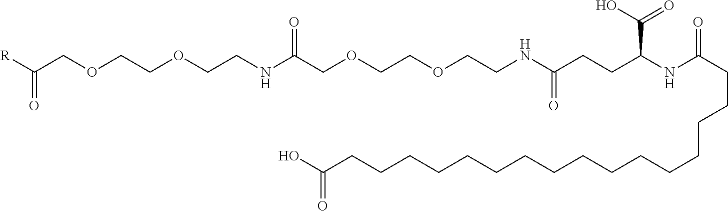 | {AEEA}2- gGlu- C18OH |
| [2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy] | |
| acetyl- | |
| 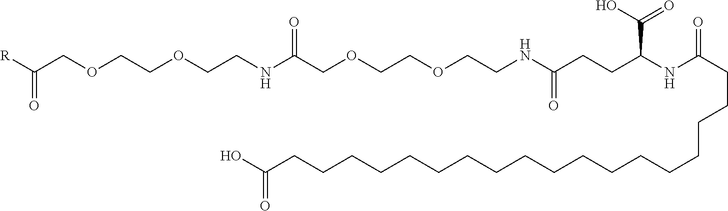 | {AEEA}2- gGlu- C20OH |
| [2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy] | |
| acetyl- | |
| 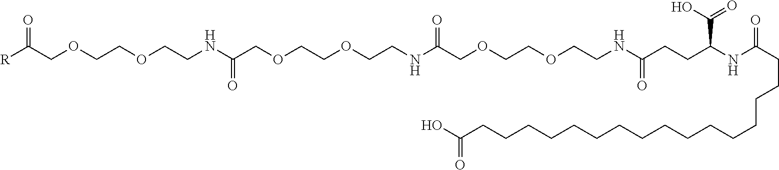 | {AEEA}3- gGlu- C18OH |
| [2-[2-[2-[[2-[2-[2-[[2-[2-[2-[(4S)-4-carboxy-4-(17-carboxyheptadecanoylamino)butanoyl]amino]ethoxy]ethoxy]acetyl]amino]ethoxy] | |
| ethoxy]acetyl]amino]ethoxy]ethoxy]acetyl- | |
| 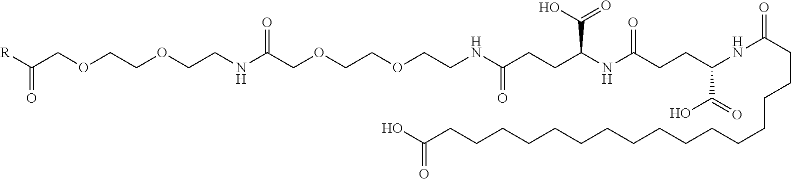 | {AEEA}2- {gGlu}2- C18OH |
| [2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]- | |
| ethoxy]acetyl]amino]ethoxy]ethoxy]acetyl- | |
| 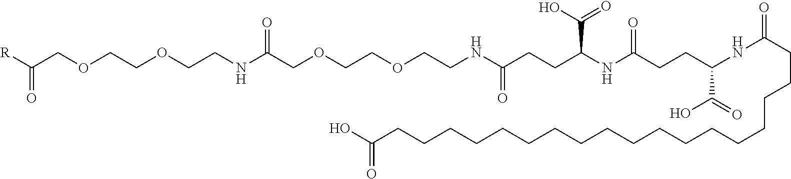 | {AEEA}2- {gGlu}2- C20OH |
| [2-[2-[2-[[2-[2-[2-[[(4S)-4-carboxy-4-[[(4S)-4-carboxy-4-(19-carboxynonadecanoylamino)butanoyl]amino]butanoyl]amino]ethoxy]- | |
| ethoxy]acetyl]amino]ethoxy]ethoxy]acetyl- | |
| 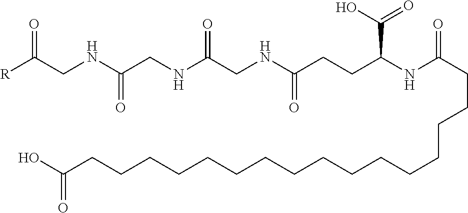 | {Gly}3- gGlu- C18OH |
| [2-[[2-[[2-[[(4S)-4-carboxy-4-(17-carboxyheptadecanoyl-amino)butanoyl]amino]acetyl]amino]acetyl]amino]acetyl]- | |
| 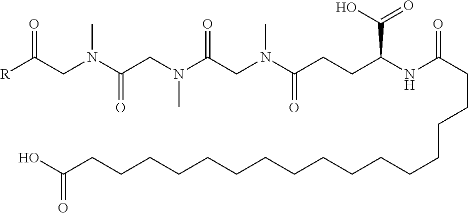 | {N- MeGly}3- gGlu- C18OH |
| [2-[methyl-[2-[methyl-[2-[methyl-[(4S)-4-carboxy-4-(17-carboxy-heptadecanoylamino)butanoyl]amino]acetyl]amino]acetyl]amino] | |
| acetyl]- | |

A further embodiment relates to compounds of formula I, wherein
X14 represents Lys wherein the —NH₂ side chain group is functionalized with a group-Z1-Z2-C(O)R⁵, wherein
Z1-Z2- represents AEEA-AEEA-gGlu and
R⁵ represents a group selected from pentadecenoyl, heptadecenoyl, nonadecanoyl, 17-carboxy-1-oxoheptadecyl, or 19-carboxy-1-oxononadecyl.

A further embodiment relates to compounds of formula I, wherein
X14 represents Lys wherein the —NH₂ side chain group is functionalized with a group-Z1-Z2-C(O)R⁵, wherein
—Z1-Z2- represents AEEA-AEEA-gGlu- and
R⁵ represents a group selected from 17-carboxy-1-oxoheptadecyl, or 19-carboxy-1-oxononadecyl.

A further embodiment relates to compounds of formula I, wherein
X14 represents Lys wherein the —NH₂ side chain group is functionalized by [2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl- or
[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl-.

A further embodiment relates to compounds of formula I, wherein
X14 represents Lys wherein the —NH₂ side chain group is functionalized by [2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl-.

A further embodiment relates to compounds of formula I, wherein
X14 represents Lys wherein the —NH₂ side chain group is functionalized with a group-Z1-Z2-C(O)R⁵, wherein
—Z1-Z2- represents AEEA-AEEA-AEEA-gGlu- and
R⁵ represents a group selected from 17-carboxy-1-oxoheptadecyl, or 19-carboxy-1-oxononadecyl.

A further embodiment relates to compounds of formula I, wherein
X14 represents Lys wherein the —NH₂ side chain group is functionalized with a group-Z1-Z2-C(O)R⁵, wherein
—Z1-Z2- represents AEEA-AEEA-gGlu-gGlu- and
R⁵ represents a group selected from 17-carboxy-1-oxoheptadecyl, or 19-carboxy-1-oxononadecyl.

A further embodiment relates to compounds of formula I, wherein
X14 represents Lys wherein the —NH₂ side chain group is functionalized by a group selected from
[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl-,
[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl-,
[2-[2-[2-[[2-[2-[2-[[2-[2-[2-[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl-,
[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]-ethoxy]acetyl]amino]ethoxy]ethoxy]acetyl-,
[2-[2-[2-[[2-[2-[2-[[(4S)-4-carboxy-4-[[(4S)-4-carboxy-4-(19-carboxynonadecanoylamino)butanoyl]amino]butanoyl]amino]ethoxy]-ethoxy]acetyl]amino]ethoxy]ethoxy]acetyl-,
[2-[[2-[[2-[[(4S)-4-carboxy-4-(17-carboxyheptadecanoyl-amino)butanoyl]amino]acetyl]amino]acetyl]amino]acetyl]-,
[2-[methyl-[2-[methyl-[2-[methyl-[(4S)-4-carboxy-4-(17-carboxy-heptadecanoylamino)butanoyl]amino]acetyl]amino]acetyl]amino]acetyl],
   R² represents NH₂,
   or a salt or solvate thereof.

A further embodiment relates to compounds of formula I, wherein
X14 represents Lys wherein the —NH₂ side chain group is functionalized by a group selected from
[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl-,
[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl-,
[2-[2-[2-[[2-[2-[2-[[2-[2-[2-[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl-,
[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]-ethoxy]acetyl]amino]ethoxy]ethoxy]acetyl-,
[2-[2-[2-[[2-[2-[2-[[(4S)-4-carboxy-4-[[(4S)-4-carboxy-4-(19-carboxynonadecanoylamino)butanoyl]amino]butanoyl]amino]ethoxy]-ethoxy]acetyl]amino]ethoxy]ethoxy]acetyl-,
[2-[[2-[[2-[[(4S)-4-carboxy-4-(17-carboxyheptadecanoyl-amino)butanoyl]amino]acetyl]amino]acetyl]amino]acetyl]-,
[2-[methyl-[2-[methyl-[2-[methyl-[(4S)-4-carboxy-4-(17-carboxy-heptadecanoylamino)butanoyl]amino]acetyl]amino]acetyl]amino]acetyl],
   R² represents OH,
   or a salt or solvate thereof.

One embodiment of the invention are compounds of the formula I,

wherein
R¹ is selected from H or methyl,
X20 is Glu,
R² is NH₂ or OH,
or a salt or solvate thereof.

One embodiment of the invention are compounds of the formula I,

wherein
R¹ is H,
X20 is Glu,
R² is NH₂ or OH,
or a salt or solvate thereof.

One embodiment of the invention are compounds of the formula I,

wherein
R¹ is H,
X20 is Glu,
R² is NH₂,
or a salt or solvate thereof.

One embodiment of the invention are compounds of the formula I,

wherein
R¹ is H,
X20 is Glu,
R² is OH,
or a salt or solvate thereof.

One embodiment of the invention are compounds of the formula I,

wherein
R¹ is methyl,
X20 is Glu,
R² is NH₂ or OH,
or a salt or solvate thereof.

One embodiment of the invention are compounds of the formula I,

wherein
R¹ is methyl,
X20 is Glu,
R² is NH₂,
or a salt or solvate thereof.

One embodiment of the invention are compounds of the formula I,

wherein
R¹ is methyl,
X20 is Glu,
R² is OH,
or a salt or solvate thereof.

One embodiment of the invention are compounds of the formula I,

wherein
R¹ is selected from H or methyl,
X20 is Aib,
R² is NH₂ or OH,
or a salt or solvate thereof.

One embodiment of the invention are compounds of the formula I,

wherein
R¹ is H,
X20 is Aib,
R² is NH₂ or OH,
or a salt or solvate thereof.

One embodiment of the invention are compounds of the formula I,

wherein
R¹ is H,
X20 is Aib,
R² is NH₂,
or a salt or solvate thereof.

One embodiment of the invention are compounds of the formula I,

wherein
R¹ is H,
X20 is Aib,
R² is OH,
or a salt or solvate thereof.

One embodiment of the invention are compounds of the formula I,

wherein
R¹ is methyl,
X20 is Aib,
R² is NH₂ or OH,
or a salt or solvate thereof.

One embodiment of the invention are compounds of the formula I,

wherein
R¹ is methyl,
X20 is Aib,
R² is NH₂,
or a salt or solvate thereof.

One embodiment of the invention are compounds of the formula I,

wherein
R¹ is methyl,
X20 is Aib,
R² is OH,
or a salt or solvate thereof.

Specific examples of compounds of formula I are the compounds of SEQ ID NO: 9-17 as well as salts or solvates thereof.

Specific examples of compounds of formula I are the compounds of SEQ ID NO: 4-8 and 18-20 as well as salts or solvates thereof.

Specific examples of compounds of formula I are the compounds of SEQ ID NO: 11 and 20 as well as salts or solvates thereof.

Specific examples of compounds of formula I are the compounds of SEQ ID NO: 8 and 13 as well as salts or solvates thereof.

Specific examples of compounds of formula I are the compounds of SEQ ID NO: 4-10 and 12-19 as well as salts or solvates thereof.

Specific examples of compounds of formula I are the compounds of SEQ ID NO: 4-17 as well as salts or solvates thereof.

Specific examples of compounds of formula I are the compounds of SEQ ID NO: 4-20 as well as salts or solvates thereof.

A specific example of compounds of formula I is the compound of SEQ ID NO: 4 as well as salts or solvates thereof.

A specific example of compounds of formula I is the compound of SEQ ID NO: 7 as well as salts or solvates thereof.

A specific example of compounds of formula I is the compound of SEQ ID NO: 9 as well as salts or solvates thereof.

A further embodiment relates to compounds of formula I, wherein the peptidic compound has at least the activity of human GIP at the GIP receptor in the HEK cell agonist assay.

A further embodiment relates to compounds of formula I, wherein the peptidic compound exhibits an activity of less than 10% compared to that of GLP-1(7-36)-amide at the GLP-1 receptor in the HEK cell agonist assay.

A further embodiment relates to compounds of formula I, wherein the peptidic compound has at least the activity of human GIP at the GIP receptor in the human adipocyte agonist assay.

A further embodiment relates to compounds of formula I, wherein the peptidic compound has at least the binding affinity of human GIP to the hGIP receptor in the HEK cell binding assay.

In a further aspect, the present invention relates to a composition comprising a compound of the invention as described herein, or a salt or solvate thereof, in admixture with a carrier.

The invention also relates to the use of a compound of the present invention for use as a medicament, particularly for the treatment of a condition as described in the specification.

The invention also relates to a composition wherein the composition is a pharmaceutically acceptable composition, and the carrier is a pharmaceutically acceptable carrier.

Peptidic Compounds of the Invention

Amino acids are referred to herein by either their name, their commonly known three letter symbols or by the one-letter symbols recommended by the IUPAC-IUB Biochemical Nomenclature Commission. Therefore, the amino acid sequences of the present invention contain the conventional one letter and three letter codes for naturally occurring amino acids, as well as generally accepted three letter codes for other amino acids, such as Aib for α-aminoisobutyric acid.

The peptidic compounds of the present invention comprise a linear backbone of amino carboxylic acids linked by peptide, i.e. carboxamide bonds. Preferably, the amino carboxylic acids are α-amino carboxylic acids and more preferably L-α-amino carboxylic acids, unless indicated otherwise, as for example D-Alanine (d-Ala or dAla). The peptidic compounds preferably comprise a backbone sequence of 39 amino carboxylic acids.

The peptidic compounds of the present invention may comprise functionalized amino acids, as for example N-methylated amino acids, e.g. N-Me-L-Tyrosine (N-MeTyr).

Amino acids within the peptide moiety (formula I) can be considered to be numbered consecutively from 1 to 39 in the conventional N-terminal to C-terminal direction. Reference to a “position” within peptidic moiety I should be constructed accordingly, as should reference to positions within native exendin-4 and other molecules, e.g., in exendin-4, His is at position 1, Gly at position 2, . . . , Met at position 14, . . . and Ser at position 39.

Peptide Synthesis

The skilled person is aware of a variety of different methods to prepare peptides. These methods include but are not limited to synthetic approaches and recombinant gene expression. Thus, one way of preparing peptides is the synthesis in solution or on a solid support and subsequent isolation and purification. A different way of preparing the peptides is gene expression in a host cell in which a DNA sequence encoding the peptide has been introduced. Alternatively, the gene expression can be achieved without utilizing a cell system. The methods described above may also be combined in any way.

A preferred way to prepare the compounds of the present invention is solid phase synthesis on a suitable resin. Solid phase peptide synthesis is a well-established methodology (see for example: Stewart and Young, Solid Phase Peptide Synthesis, Pierce Chemical Co., Rockford, III., 1984; E. Atherton and R. C. Sheppard, Solid Phase Peptide Synthesis. A Practical Approach, Oxford-IRL Press, New York, 1989). Solid phase synthesis is initiated by attaching an N-terminally protected amino acid with its carboxy terminus to an inert solid support carrying a cleavable linker. This solid support can be any polymer that allows coupling of the initial amino acid, e.g. a trityl resin, a chlorotrityl resin, a Wang resin or a Rink resin in which the linkage of the carboxy group (or carboxamide for Rink resin) to the resin is sensitive to acid (when Fmoc strategy is used). The polymer support must be stable under the conditions used to deprotect the α-amino group during the peptide synthesis.

After the N-terminally protected first amino acid has been coupled to the solid support, the α-amino protecting group of this amino acid is removed. The remaining protected amino acids are then coupled one after the other or with a preformed dipeptide, tripeptide or tetrapeptide or with an amino acid building block with a modified sidechain as e.g. N-alpha-(9-fluorenylmethyloxycarbonyl)-N-epsilon-(N-alpha′-palmitoyl-L-glutamic-acid alpha′-t-butyl ester)-L-lysine in the order represented by the peptide sequence using appropriate amide coupling reagents, for example BOP, HBTU, HATU or DIC/HOBt/HOAt, wherein BOP, HBTU and HATU are used with tertiary amine bases. Alternatively, the liberated N-terminus can be functionalized with groups other than amino acids, for example carboxylic acids, etc.

Usually, reactive side-chain groups of the amino acids are protected with suitable blocking groups. These protecting groups are removed after the desired peptides have been assembled. They are removed concomitantly with the cleavage of the desired product from the resin under the same conditions. Protecting groups and the procedures to introduce protecting groups can be found in Protective Groups in Organic Synthesis, 3d ed., Greene, T. W. and Wuts, P. G. M., Wiley & Sons (New York: 1999).

In some cases, it might be desirable to have side chain protecting groups that can selectively be removed while other side chain protecting groups remain intact. In this case the liberated functionality can be selectively functionalized. For example, a lysine may be protected with an ivDde ([1-(4,4-dimethyl-2,6-dioxocyclohex-1-ylidene)-3-methylbutyl) protecting group (S. R. Chhabra et al., Tetrahedron Lett. 39, (1998), 1603) which is labile to a very nucleophilic base, for example 4% hydrazine in DMF (dimethyl formamide). Thus, if the N-terminal amino group and all side-chain functionalities are protected with acid labile protecting groups, the ivDde group can be selectively removed using 4% hydrazine in DMF and the corresponding free amino group can then be further modified, e.g. by acylation.

For example, a lysine may be protected with an Mmt [(4-methoxyphenyl)diphenylmethyl] protecting group (G. M. Dubowchik et al., Tetrahedron Lett. 1997, 38(30), 5257) which is labile to very mild acid, for example acetic acid and trifluoroethanol in dichloromethane. Thus, if the N-terminal amino group and all side-chain functionalities are protected with protecting groups only labile to strong acids, the Mmt group can be selectively removed using a mixture of acetic acid and trifluoroethanol in dichloromethane (1:2:7) and the corresponding free amino group can then be further modified, e.g. by acylation.

The lysine can alternatively be coupled to a protected amino acid and the amino group of this amino acid can then be deprotected resulting in another free amino group which can be acylated or attached to further amino acids. Alternatively, the side chain (as described in table 1) can be introduced together with the lysine during peptide synthesis using a prefunctionalized building block, e.g. N-alpha-(9-fluorenylmethyloxycarbonyl)-N-epsilon-(N-alpha′-palmitoyl-L-glutamic-acid alpha′-t-butyl ester)-L-lysine or Fmoc-L-Lys[{AEEA}2-gGlu(OtBu)-C18OtBu]-OH [=(2S)-6-[[2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetyl]amino]-2-(9H-fluoren-9-ylmethoxycarbonyl-amino)hexanoic acid (CAS Registry Number 1662688-20-1)], as coupling partner.

Finally, the peptide is cleaved from the resin. This can be achieved by using King's cocktail (D. S. King, C. G. Fields, G. B. Fields, Int. J. Peptide Protein Res. 36, 1990, 255-266) or similar cleavage cocktails known to the person skilled in the art. For example, EDT can be replaced by DODT or a mixture of TIS, water and TFA can be used. The raw material can then be purified by chromatography, e.g. preparative RP-HPLC, if necessary.

Potency

As used herein, the term “potency” or “in vitro potency” is a measure for the ability of a compound to activate the receptors for GLP-1, GIP or glucagon in a cell-based assay. Numerically, it is expressed as the “EC50 value”, which is the effective concentration of a compound that induces a half maximal increase of response (e.g. formation of intracellular cAMP) in a dose-response experiment.

Therapeutic Uses

The peptidic incretin hormones GLP-1 and GIP are secreted by intestinal enteroendocrine cells in response to food and account for up to 70% of meal-stimulated insulin secretion. The receptor for GIP is broadly expressed in peripheral tissues including pancreatic islets, adipose tissue, stomach, small intestine, heart, bone, lung, kidney, testis, adrenal cortex, pituitary, endothelial cells, trachea, spleen, thymus, thyroid and brain. Consistent with its biological function as incretin hormone, the pancreatic B-cells express the highest levels of the receptor for GIP in humans.

There is some clinical evidence that the GIP-receptor mediated signaling could be impaired in patients with T2DM but the impairment of GIP-action is shown to be reversible and could be restored with improvement of the diabetic status. Of note, the stimulation of insulin secretion by GIP is strictly glucose-dependent ensuring a fail-safe mechanism associated with a low risk for hypoglycemia.

At the pancreatic beta cell level, GIP has been shown to promote glucose sensitivity, neogenesis, proliferation, transcription of proinsulin and hypertrophy, as well as anti-apoptosis.

Further GIP actions in peripheral tissues beyond the pancreas comprise increased bone formation and decreased bone resorption as well as neuroprotective effects which might be beneficial for the treatment of osteoporosis and cognitive defects like Alzheimer's disease.

As GLP-1 and GIP are known for their anti-diabetic effects, and GLP-1 is known for its food intake-suppressing effects, it is conceivable that a combination of the activities of the two hormones can yield a powerful medication for treatment of the metabolic syndrome and in particular its components diabetes and obesity. Stimulating both, the GLP-1 and the GIP receptor could be anticipated to have additive or synergistic anti-diabetic benefit.

Thus, targeting of the GIP receptor with suitable agonists alone or on top of GLP-1R agonists offers an attractive approach for treatment of metabolic disorders, including diabetes.

Accordingly, the compounds of the invention may be used for treatment of glucose intolerance, insulin resistance, pre-diabetes, increased fasting glucose (hyperglycemia), type 2 diabetes, hypertension, dyslipidemia, arteriosclerosis, coronary heart disease, peripheral artery disease, stroke or any combination of these individual disease components.

In addition, they may be used for control of appetite, feeding and caloric intake, prevention of weight gain, promotion of weight loss, reduction of excess body weight and altogether treatment of obesity, including morbid obesity.

The compounds of the invention are agonists of the GIP receptor and may provide therapeutic benefit to address a clinical need for targeting the metabolic syndrome by allowing simultaneous treatment of diabetes and obesity.

Further disease states and health conditions which could be treated with the compounds of the invention are obesity-linked inflammation, obesity-linked gallbladder disease and obesity-induced sleep apnoea.

Although all these conditions could be associated directly or indirectly with obesity, the effects of the compounds of the invention may be mediated in whole or in part via an effect on body weight, or independent thereof.

The compounds of the present invention may be particularly effective in improving glycaemic control and reducing body weight when they are administered in combination with a GLP-1 receptor agonist (as part of the same pharmaceutical formulation or as separate formulations).

Further, diseases to be treated may be neurodegenerative diseases such as Alzheimer's disease or Parkinson's disease, or other degenerative diseases as described above.

The compounds of the present invention may also be used for the treatment and/or prevention of any of the diseases, disorders, or conditions associated with diabetes-related osteoporosis or osteoporosis including increased risk of bone fractures (N. B. Khazai et al, Current Opinion in Endocrinology, Diabetes and Obesity 2009, 16(6), 435).

In one embodiment the compounds are useful in the treatment or prevention of hyperglycemia, type 2 diabetes, and/or obesity.

The compounds of the invention have the ability to reduce blood glucose level, and/or to reduce HbA1c levels of a patient. These activities of the compounds of the invention can be assessed in animal models known to the skilled person and described herein in the Methods and in examples.

The compounds of the invention may have the ability to reduce body weight of a patient. These activities of the compounds of the invention can be assessed in animal models known to the skilled person.

The compounds of the invention may be useful in the treatment or prevention of hepatosteatosis, preferably non-alcoholic liver-disease (NAFLD) and non-alcoholic steatohepatitis (NASH). These activities of the compounds of the invention can be assessed in animal models known to the skilled person.

The compounds of the invention may have the ability to reduce nausea of a patient and avoid vomiting. These activities of the compounds of the invention can be assessed in animal models known to the skilled person.

By “treat” or “treating” is meant to administer a compound or composition or a combination of compounds or compositions to a subject in order to eliminate a disease or disorder; arrest or slow a disease or disorder in a subject; inhibit or slow the development of a new disease or disorder in a subject; decrease the frequency or severity of symptoms and/or recurrences in a subject who currently has or who previously has had a disease or disorder; and/or prolong, i.e., increase, the lifespan of the subject. In particular, the term “treating/treatment of a disease or disorder” includes curing, shortening the duration, ameliorating, slowing down or inhibiting progression or worsening of a disease or disorder or the symptoms thereof.

By “prevent” or “preventing” is particularly meant to administer a compound or composition or a combination of compounds or compositions to a subject in order to inhibit or delay the onset of a disease or disorder in a subject.

The term “subject” means according to the invention a subject for treatment, in particular a diseased subject (also referred to as “patient”), including human beings, non-human primates or other animals, in particular mammals, such as cows, horses, pigs, sheep, goats, dogs, cats, rabbits or rodents, such as mice, rats, guinea pigs and hamsters. In one embodiment, the subject/patient is a human being.

The compounds of formula I are particularly suitable for the treatment or prevention of diseases or disorders caused by, associated with and/or accompanied by disturbances in carbohydrate and/or lipid metabolism, e.g. for the treatment or prevention of hyperglycemia, type 2 diabetes, impaired glucose tolerance, type 1 diabetes, obesity and metabolic syndrome. Further, the compounds of the invention may be suitable for the treatment or prevention of degenerative diseases, particularly neurodegenerative diseases. Further, the compounds of the invention may be useful for the treatment or prevention of diseases accompanied by nausea or vomiting, or as an anti-emetic agent for nausea or vomiting.

The compounds described find use, inter alia, in preventing weight gain or promoting weight loss. By “preventing” is meant inhibiting or reducing when compared to the absence of treatment and is not necessarily meant to imply complete cessation of a disorder.

Independently of their effect on body weight, the compounds of the invention may have a beneficial effect on circulating cholesterol levels, being capable of improving lipid levels, particularly LDL, as well as HDL levels (e.g. increasing HDL/LDL ratio).

Thus, the compounds of the invention may be used for direct or indirect therapy of any condition caused or characterised by excess body weight, such as the treatment and/or prevention of obesity, morbid obesity, obesity linked inflammation, obesity linked gallbladder disease, obesity induced sleep apnoea. They may also be used for treatment and prevention of the metabolic syndrome, diabetes, hypertension, atherogenic dyslipidemia, atherosclerosis, arteriosclerosis, coronary heart disease, or stroke. Their effects in these conditions may be as a result of or associated with their effect on body weight or may be independent thereof.

Medical uses include delaying or preventing disease progression in type 2 diabetes, treating metabolic syndrome, treating obesity or preventing overweight, for decreasing food intake, reducing body weight, delaying the progression from impaired glucose tolerance (IGT) to type 2 diabetes; delaying the progression from type 2 diabetes to insulin-requiring diabetes and hepatic steatosis.

The term “disease or disorder” refers to any pathological or unhealthy state, in particular obesity, overweight, metabolic syndrome, diabetes mellitus, hyperglycemia, dyslipidemia and/or atherosclerosis.

The term “metabolic syndrome” can be defined as a clustering of at least three of the following medical conditions: abdominal (central) obesity (e.g., defined as waist circumference >94 cm for Europid men and >80 cm for Europid women, with ethnicity specific values for other groups), elevated blood pressure (e.g., 130/85 mmHg or higher), elevated fasting plasma glucose (e.g., at least 100 mg/dL), high serum triglycerides (e.g., at least 150 mg/dL), and low high-density lipoprotein (HDL) levels (e.g., less than 40 mg/dL for males and less than 50 mg/dL for females).

Obesity is a medical condition in which excess body fat has accumulated to the extent that it may have an adverse effect on health and life expectancy and due to its increasing prevalence in adults and children it has become one of the leading preventable causes of death in modern world. It increases the likelihood of various other diseases, including heart disease, type 2 diabetes, obstructive sleep apnoea, certain types of cancer, as well as osteoarthritis, and it is most commonly caused by a combination of excess food intake, reduced energy expenditure, as well as genetic susceptibility.

In terms of a human (adult) subject, obesity can be defined as a body mass index (BMI) greater than or equal to 30 kg/m2 (BMI>30 kg/m2). The BMI is a simple index of weight-for-height that is commonly used to classify overweight and obesity in adults. It is defined as a person's weight in kilograms divided by the square of his/her height in meters (kg/m2).

The term “overweight” refers to a medical condition in which the amount of body fat is higher than is optimally healthy. In terms of a human (adult) subject, obesity can be defined as a body mass index (BMI) greater than or equal to 25 kg/m2 (e.g., 25 kg/m2<BMI<30 kg/m2).

Diabetes mellitus, often simply called diabetes, is a group of metabolic diseases in which a person has high blood sugar levels, either because the body does not produce enough insulin, or because cells do not respond to the insulin that is produced. The most common types of diabetes are: (1) type 1 diabetes, where the body fails to produce insulin; (2) type 2 diabetes (T2DM), where the body fails to use insulin properly, combined with an increase in insulin deficiency over time, and (3) gestational diabetes, where women develop diabetes due to their pregnancy. All forms of diabetes increase the risk of long-term complications, which typically develop after many years. Most of these long-term complications are based on damage to blood vessels and can be divided into the two categories “macrovascular” disease, arising from atherosclerosis of larger blood vessels and “microvascular” disease, arising from damage of small blood vessels. Examples for macrovascular disease conditions are ischemic heart disease, myocardial infarction, stroke and peripheral vascular disease. Examples for microvascular diseases are diabetic retinopathy, diabetic nephropathy, as well as diabetic neuropathy.

The current WHO diagnostic criteria for diabetes mellitus are as follows: fasting plasma glucose 15≥7.0 mmol/l (126 mg/dL) or 2-h plasma glucose ≥11.1 mmol/l (200 mg/dL).

The term “hyperglycemia” refers to an excess of sugar (glucose) in the blood, e.g. above 11.1 mmol/l (200 mg/dl).

The term “hypoglycemia” refers to a blood glucose level below normal levels, e.g below 3.9 mmol/L (70 mg/dL).

The term “dyslipidemia” refers to a disorder of lipoprotein metabolism, including lipoprotein overproduction (“hyperlipidemia”) or deficiency (“hypolipidemia”). Dyslipidemias may be manifested by elevation of the total cholesterol, low-density lipoprotein (LDL) cholesterol and/or triglyceride concentrations, and/or a decrease in high-density lipoprotein (HDL) cholesterol concentration in the blood.

“Atherosclerosis” is a vascular disease characterized by irregularly distributed lipid deposits called plaque in the intima of large and medium-sized arteries that may cause narrowing of arterial lumens and proceed to fibrosis and calcification. Lesions are usually focal and progress slowly and intermittently. Occasionally plaque rupture occurs leading to obstruction of blood flow resulting in tissue death distal to the obstruction. Limitation of blood flow accounts for most clinical manifestations, which vary with the distribution and severity of the obstruction.

The compounds of formula I are particularly suitable as a suppressant for “vomiting” or “nausea”.

The compounds of formula I are particularly suitable for the treatment or prevention where the vomiting or the nausea is caused by one or more conditions or causes selected from the following (I) to (6):

(I) diseases such as gastroparesis, gastrointestinal hypomotility, peritonitis, abdominal tumor, constipation, gastrointestinal obstruction, cyclic vomiting syndrome, chronic unexplained nausea and vomiting, acute and chronic pancreatitis, hyperkalemia, cerebral edema, intracranial lesion, metabolic disorder, gastritis caused by an infection, postoperative disease, myocardial infarction, migraine, intracranial hypertension, and intracranial hypotension (e. g., altitude sickness);

(2) drugs such as (i) alkylating agents (e. g., cyclophosphamide, carmustine, lomustine, chlorambucil, streptozocin, dacarbazine, ifosfamide, temozolomide, busulfan, bendamustine, and meiphaian), cytotoxic antibiotics (e. g., dactinomycin, doxorubicin, mitomycin-C, bleomycin, epirubicin, actinomycin D, amrubicin, idarubicin, daunorubicin, and pirarubicin), antimetabolic agents (e. g., cytarabine, methotrexate, S-fluorouraci, enocitabine, and ciofarabine), vinca alkaloids (e. g., etoposide, vinblastine, and vincristine), other chemotherapeutic agents such as cisplatin, procarbazine, hydroxyurea, azacytidine, irinotecan, interferon u, interleukin-2, oxaliplatin, carboplatin, nedaplatin, and miriplatin; (ii) opioid analgesics (e. g., morphine); (iii) dopamine receptor D1D2 agonists (e. g., apomorphine); (iv) cannabis and cannabinoid products including cannabis hyperemesis syndrome

(3) radiation sickness or radiation therapy for the chest, the abdomen, or the like used to treat cancers;

(4) a poisonous substance or a toxin;

(5) pregnancy including hyperemesis gravidarium; and

(6) a vestibular disorder such as motion sickness or dizziness.

Additionally, the compound of the present invention may be used as a preventive/therapeutic agent for chronic unexplained nausea and vomiting. The vomiting or nausea also includes imminent unpleasant sensations of wanting to eject the contents of the stomach through the mouth such as feeling queasy and retching and may also be accompanied by autonomic symptoms such as facial pallor, cold sweat, salivary secretion, tachycardia, and diarrhea. The vomiting also includes acute vomiting, protracted vomiting, and anticipatory vomiting.

Pharmaceutical Compositions

In a further aspect, the present invention relates to a composition comprising a compound of the invention in admixture with a carrier. In preferred embodiments, the composition is a pharmaceutically acceptable composition and the carrier is a pharmaceutically acceptable carrier. The compounds of the invention may be in the form of a salt, e.g. a pharmaceutically acceptable salt or a solvate, e.g. a hydrate. In still a further aspect, the present invention relates to a composition for use in a method of medical treatment, particularly in human medicine.

The term “pharmaceutical composition” indicates a mixture containing ingredients that are compatible when mixed and which may be administered. A pharmaceutical composition may include one or more medicinal drugs. Additionally, the pharmaceutical composition may include carriers, buffers, acidifying agents, alkalizing agents, solvents, adjuvants, tonicity adjusters, emollients, expanders, preservatives, physical and chemical stabilizers e.g. surfactants, antioxidants and other components, whether these are considered active or inactive ingredients. Guidance for the skilled in preparing pharmaceutical compositions may be found, for example, in Remington: The Science and Practice of Pharmacy, (20th ed.) ed. A. R. GennaroA. R., 2000, Lippencott Williams & Wilkins and in R. C. Rowe et al. (Ed), Handbook of Pharmaceutical Excipients, PhP, May 2013 update.

The exendin-4 peptide derivatives of the present invention, or salts thereof, are administered in conjunction with a pharmaceutically acceptable carrier, diluent, or excipient as part of a pharmaceutical composition.

A “pharmaceutically acceptable carrier” is a carrier which is physiologically acceptable (e.g. physiologically acceptable pH) while retaining the therapeutic properties of the substance with which it is administered. Standard acceptable pharmaceutical carriers and their formulations are known to one skilled in the art and described, for example, in Remington: The Science and Practice of Pharmacy, (20th ed.) ed. A. R. GennaroA. R., 2000, Lippencott Williams & Wilkins and in R. C. Rowe et al. (Ed), Handbook of Pharmaceutical excipients, PhP, May 2013 update. One exemplary pharmaceutically acceptable carrier is physiological saline solution.

In one embodiment carriers are selected from the group of buffers (e.g. citrate/citric acid, acetate/acetic acid, phosphate/phosphoric acid), acidifying agents (e.g. hydrochloric acid), alkalizing agents (e.g. sodium hydroxide), preservatives (e.g. phenol, m-cresol, benzylic alcohol), co-solvents (e.g. polyethylene glycol 400), tonicity adjusters (e.g. mannitol, glycerol, sodium chloride, propylene glycol), stabilizers (e.g. surfactant, antioxidants, amino acids).

Concentrations used are in a range that is physiologically acceptable.

Acceptable pharmaceutical carriers or diluents include those used in formulations suitable for oral, rectal, nasal or parenteral (including subcutaneous, intramuscular, intravenous, intradermal, and transdermal) administration. The compounds of the present invention will typically be administered parenterally.

The term “pharmaceutically acceptable salt” means salts of the compounds of the invention which are safe and effective for use in mammals, e.g. acetate salts, chloride salts or sodium salts.

The term “solvate” means complexes of the compounds of the invention or salts thereof with solvent molecules, e.g. organic solvent molecules and/or water.

In the pharmaceutical composition, the exendin-4 derivative can be in monomeric or oligomeric form.

The term “therapeutically effective amount” of a compound refers to a nontoxic but sufficient amount of the compound to provide the desired effect. The amount of a compound of the formula I necessary to achieve the desired biological effect depends on a number of factors, for example the specific compound chosen, the intended use, the mode of administration and the clinical condition of the patient. An appropriate “effective” amount in any individual case may be determined by one of ordinary skill in the art using routine experimentation. For example, the “therapeutically effective amount” of a compound of the formula I is about 0.01 to 100 mg/dose, preferably 1 to 30 mg/dose.

Pharmaceutical compositions of the invention are those suitable for parenteral (for example subcutaneous, intramuscular, intradermal or intravenous), rectal, topical and peroral (for example sublingual) administration, although the most suitable mode of administration depends in each individual case on the nature and severity of the condition to be treated and on the nature of the compound of formula I used in each case. In one embodiment, application is parenteral, e.g. subcutaneous.

In case of parenteral application, it could be favorable for the corresponding formulations to include at least one antimicrobial preservative in order to inhibit the growth of microbes and bacteria between administrations. In case of parenteral application, it would be mandatory for the corresponding formulations to include at least one antimicrobial preservative in order to inhibit the growth of microbes and bacteria between administrations when using a multi-dose device. Preferred preservatives are benzylic alcohol or phenolic compounds like phenol or m-cresol. It has been described that these ingredients can induce aggregation for peptides and proteins leading to lower solubility and stability in the formulation (see Bis et al., Int. J. Pharm. 2014, 472, 356; Kamerzell, Adv. Drug Deliv. Rev. 2011, 63, 1118).

Administration Unit, Package, (Pen) Device and Administration

The compound(s) of the present invention can be prepared for use in suitable pharmaceutical compositions. The suitable pharmaceutical compositions may be in the form of one or more administration units.

The compositions may be prepared by any suitable pharmaceutical method which includes a step in which the compound(s) of the present invention and the carrier (which may consist of one or more additional ingredients) are brought into contact. The administration units may be for example capsules, tablets, dragées, granules sachets, drops, solutions, suspensions, lyophylisates and powders, each of which contains a defined amount of the compound(s) of the present invention.

Each of the above-mentioned administration units of the compound(s) of the invention or pharmaceutical composition of the invention (administration units) may be provided in a package for easy transport and storage. The administration units are packaged in standard single or multi-dosage packaging, their form, material and shape depending on the type of units prepared.

In some embodiments, the present invention provides kits that comprise a compound of formula (I), in any of its stereoisomeric forms, or a physiologically acceptable salt or solvate thereof, and a set of instructions relating to the use of the compound for the methods described herein. In some embodiments, the kit further comprises one or more inert carriers and/or diluents. In some embodiments, the kit further comprises one or more other pharmacologically active compounds, such as those described herein.

In certain embodiments administration units may be provided together with a device for application, for example together with a syringe, an injection pen or an autoinjector. Such devices may be provided separate from a pharmaceutical composition or prefilled with the pharmaceutical composition.

A “pen-type injection device”, often briefly referred to as “injection pen”, is typically an injection device having an elongated shape that resembles to a fountain pen for writing. Although such pens usually have a tubular cross-section, they could easily have a different cross-section such as triangular, rectangular or square or any variation around these geometries. Generally, pen-type injection devices comprise three primary elements: a cartridge section that includes a cartridge often contained within a housing or holder; a needle assembly connected to one end of the cartridge section; and a dosing section connected to the other end of the cartridge section. The cartridge, often also referred to as “ampoule”, typically includes a reservoir that is filled with a medication, a movable rubber type bung or stopper located at one end of the cartridge reservoir, and a top having a pierceable rubber seal located at the other, often necked-down, end. A crimped annular metal band is typically used to hold the rubber seal in place. While the cartridge housing may be typically made of plastic, cartridge reservoirs have historically been made of glass.

Combination Therapy

The compounds of formula I are suitable for human treatment without an additional therapeutically effective agent. In other embodiments, however, the compounds may be used together with at least one additional therapeutically active agent, as described in “combination therapy”.

The compounds of the present invention, agonists for the GIP receptor, can be widely combined with other pharmacologically active compounds, such as all drugs mentioned in the Rote Liste 2017, e.g. with all antidiabetics mentioned in the Rote Liste 2016, chapter 12, all weight-reducing agents or appetite suppressants mentioned in the Rote Liste 2017, chapter 6, all lipid-lowering agents mentioned in the Rote Liste 2017, chapter 58, all antihypertensives and nephroprotectives, mentioned in the Rote Liste 2017, chapter 17, and all diuretics mentioned in the Rote Liste 2017, chapter 36.

The active ingredient combinations can be used especially for a synergistic improvement in action. They can be applied either by separate administration of the active ingredients to the patient or in the form of combination products in which a plurality of active ingredients are present in one pharmaceutical preparation. The amount of the compound of the invention and the other pharmaceutically active ingredient(s) and the relative timings of administration will be selected in order to achieve the desired combined therapeutic effect. The administration of the combination may be concomitantly in: (1) a unitary pharmaceutical composition including all pharmaceutically active ingredients; or (2) separate pharmaceutical compositions each including at least one of the pharmaceutically active ingredients. Alternatively, the combination may be administered separately in a sequential manner wherein one treatment agent is administered first and the other treatment agent is administered second, or vice versa. When the active ingredients are administered by separate administration of the active ingredients, this can be done simultaneously or successively.

Other active substances which are suitable for such combinations include in particular those which for example potentiate the therapeutic effect of one or more active substances with respect to one of the indications mentioned and/or which allow the dosage of one or more active substances to be reduced.

Most of the active ingredients mentioned hereinafter are disclosed in the USP Dictionary of USAN and International Drug Names, US Pharmacopeia, Rockville 2014.

Therapeutic agents which are suitable for combinations include, for example, antidiabetic agents such as:

Insulin and insulin derivatives, for example: insulin glargine (e.g. Lantus®), higher than 100 U/ml concentrated insulin glargine, e.g. 270-330 U/ml of insulin glargine or 300 U/ml of insulin glargine (e.g. Toujeo®), insulin glulisine (e.g. Apidra®), insulin detemir (e.g. Levemir®), insulin lispro (e.g. Humalog®, Liprolog®), insulin degludec (e.g. DegludecPlus®, IdegLira (NN9068)), insulin aspart and aspart formulations (e.g. NovoLog®), basal insulin and analogues (e.g. LY2605541, LY2963016, NN1436), PEGylated insulin lispro (e.g. LY-275585), long-acting insulins (e.g. NN1436, Insumera (PE0139), AB-101, AB-102, Sensulin LLC), intermediate-acting insulins (e.g. Humulin®N, Novolin®N), fast-acting and short-acting insulins (e.g. Humulin®R, Novolin®R, Linjeta® (VIAject®), PH20 insulin, NN1218, HinsBet®, premixed insulins, SuliXen®, NN1045, insulin plus Symlin®, PE-0139, ACP-002 hydrogel insulin, and oral, inhalable, transdermal and buccal or sublingual insulins (e.g. Exubera®, Nasulin®, Afrezza®, insulin tregopil, TPM-02 insulin, Capsulin®, Oral-lyn®, Cobalamin® oral insulin, ORMD-0801, Oshadi oral insulin, NN1953, NN1954, NN1956, VIAtab®).

Also suitable are those insulin derivatives which are bonded to albumin or another protein by a bifunctional linker.

GLP-1, GLP-1 analogues and GLP-1 receptor agonists, for example: lixisenatide (e.g. Lyxumia®), exenatide (e.g. exendin-4, rExendin-4, Byetta®, Bydureon®, exenatide NexP), liraglutide (e.g. Victoza®), semaglutide (e.g. Ozempic®), taspoglutide, albiglutide, dulaglutide (e.g. Trulicity®), ACP-003, CJC-1134-PC, GSK-2374697, PB-1023, TTP-054, efpeglenatide (HM-11260C), CM-3, GLP-1 Eligen, AB-201, ORMD-0901, NN9924, NN9926, NN9927, Nodexen, Viador-GLP-1, CVX-096, ZYOG-1, ZYD-1, ZP-3022, CAM-2036, DA-3091, DA-15864, ARI-2651, ARI-2255, exenatide-XTEN (VRS-859), exenatide-XTEN+Glucagon-XTEN (VRS-859+AMX-808) and polymer-bound GLP-1 and GLP-1 analogues.

Dual GLP-1/glucagon receptor agonists, e.g. BHM-034, OAP-189 (PF-05212389, TKS-1225), pegapamodutide (TT-401/402), ZP2929, JNJ64565111 (HM 12525A, LAPS-HMOXM25), MOD-6030, NN9277, LY-3305677, MEDI-0382, MK8521, BI456906, VPD-107, H&D-001A, PB-718, SAR425899 or compounds disclosed in WO2014/056872.

Dual GLP-1/GIP agonists, e.g. RG-7685 (MAR-701), RG-7697 (MAR-709, NN9709), BHM081, BHM089, BHM098, LBT-6030, ZP-1-70), TAK-094, SAR438335, Tirzepatide (LY3298176) or compounds disclosed in WO2014/096145, WO2014/096148, WO2014/096149, WO2014/096150 and WO2020/023386.

Triple GLP-1/glucagon/GIP receptor agonists (e.g. Tri-agonist 1706 (NN9423), HM15211).

Dual GLP-1R agonist/Proprotein convertase subtilisin/kexin type 9 (e.g. MEDI-4166).

Dual GLP-1/GLP-2 receptor agonists (e.g. ZP-GG-72).

Dual GLP-1/gastrin agonists (e.g. ZP-3022).

Other suitable combination partners are:

Further gastrointestinal peptides such as peptide YY 3-36 (PYY3-36) or analogues thereof and pancreatic polypeptide (PP) or analogues thereof (e.g. PYY 1562 (NN9747/NN9748)).

Calcitonin and calcitonin analogs, amylin and amylin analogues (e.g. pramlintide, Symlin®), dual calcitonin and amylin receptor agonists such as Salmon Calcitonin (e.g. Miacalcic®), davalintide (AC2307), mimylin, AM833 (NN9838), KBP-042, KBP-088, and KBP-089, ZP-4982/ZP-5461, elcatonin.

Glucagon-like-peptide 2 (GLP-2), GLP-2 analogues, and GLP-2 receptor agonists, for example: teduglutide (e.g. Gattex®), elsiglutide, glepaglutide, FE-203799, HM15910.

Glucagon receptor agonists (e.g. G530S (NN9030), dasiglucagon, HM15136, SAR438544, DIO-901, AMX-808) or antagonists, glucose-dependent insulinotropic polypeptide (GIP) receptor agonists (e.g. ZP-1-98, AC163794) or antagonists (e.g. GIP(3-30)NH₂), ghrelin antagonists or inverse agonists, xenin and analogues thereof.

Human fibroblast growth factor 21 (FGF21) and derivatives or analogues such as LY2405319 and NN9499 or other variants of FGF21.

Dipeptidyl peptidase-IV (DPP-4) inhibitors, for example: alogliptin (e.g. Nesina®, Kazano®), linagliptin (e.g. Ondero®, Trajenta®, Tradjenta®, Trayenta®), saxagliptin (e.g. Onglyza®, Komboglyze XR®), sitagliptin (e.g. Januvia®, Xelevia®, Tesavel®, Janumet®, Velmetia®, Juvisync®, Janumet XR®), anagliptin, teneligliptin (e.g. Tenelia®), trelagliptin, vildagliptin (e.g. Galvus®, Galvumet®), gemigliptin, omarigliptin, evogliptin, dutogliptin, DA-1229, MK-3102, KM-223, KRP-104, PBL-1427, Pinoxacin hydrochloride, and Ari-2243.

Sodium-dependent glucose transporter 2 (SGLT-2) inhibitors, for example: Canagliflozin (e.g. Invokana®), Dapagliflozin (e.g. Forxiga®), Remogliflozin, Sergliflozin, Empagliflozin (e.g. Jardiance®), Ipragliflozin, Tofogliflozin, Luseogliflozin, Ertuglifozin/PF-04971729, RO-4998452, Bexagliflozin (EGT-0001442), SBM-TFC-039, Henagliflozin (SHR3824), Janagliflozin, Tianagliflozin, AST1935, JRP493, HEC-44616.

Dual inhibitors of SGLT-1 and SGLT-2 (e.g. sotagliflozin, LX-4211, LIK066), SGLT-1 inhibitors (e.g. LX-2761, Mizagliflozin (KGA-3235)) or SGLT-1 inhibitors in combination with anti-obesity drugs such as ileal bile acid transfer (IBAT) inhibitors (e.g. GSK-1614235 and GSK-2330672).

Biguanides (e.g. Metformin, Buformin, Phenformin).

Thiazolidinediones (e.g. Pioglitazone, Rivoglitazone, Rosiglitazone, Troglitazone), glitazone analogues (e.g. Iobeglitazone).

Peroxisome proliferator-activated receptors (PPAR-)(alpha, gamma or alpha/gamma) agonists or modulators (e.g. saroglitazar (e.g. Lipaglyn®), GFT-505), or PPAR gamma partial agonists (e.g. Int-131).

Sulfonylureas (e.g. Tolbutamide, Glibenclamide, Glimepiride (e.g. Amaryl®), Glipizide), Meglitinides (e.g. Nateglinide, Repaglinide, Mitiglinide)

Alpha-glucosidase inhibitors (e.g. Acarbose, Miglitol, Voglibose).

GPR119 agonists (e.g. GSK-1292263, PSN-821, MBX-2982, APD-597, ARRY-981, ZYG-19, DS-8500, HM-47000, YH-Chem1, YH18421, DA-1241).

GPR40 agonists (e.g. TUG-424, P-1736, P-11187, JTT-851, GW9508, CNX-011-67, AM-1638, AM-5262).

GPR120 agonists and GPR142 agonists.

Systemic or low-absorbable TGR5 (GPBAR1=G-protein-coupled bile acid receptor 1) agonists (e.g. INT-777, XL-475, SB756050).

Diabetes immunotherapeutics, for example: oral C—C chemokine receptor type 2 (CCR-2) antagonists (e.g. CCX-140, JNJ-41443532), interleukin 1 beta (IL-1ß) antagonists (e.g. AC-201), or oral monoclonal antibodies (MoA) (e.g. methalozamide, VVP808, PAZ-320, P-1736, PF-05175157, PF-04937319).

Anti-inflammatory agents for the treatment of the metabolic syndrome and diabetes, for example: nuclear factor kappa B inhibitors (e.g. Triolex®).

Adenosine monophosphate-activated protein kinase (AMPK) stimulants, for example: Imeglimin (PXL-008), Debio-0930 (MT-63-78), R-118.

Inhibitors of 11-beta-hydroxysteroid dehydrogenase 1 (11-beta-HSD-1) (e.g. LY2523199, BMS770767, RG-4929, BMS816336, AZD-8329, HSD-016, BI-135585).

Activators of glucokinase (e.g. PF-04991532, TTP-399 (GK1-399), GKM-001 (ADV-1002401), ARRY-403 (AMG-151), TAK-329, TMG-123, ZYGK1).

Inhibitors of diacylglycerol O-acyltransferase (DGAT) (e.g. pradigastat (LCQ-908)), inhibitors of protein tyrosine phosphatase 1 (e.g. trodusquemine), inhibitors of glucose-6-phosphatase, inhibitors of fructose-1,6-bisphosphatase, inhibitors of glycogen phosphorylase, inhibitors of phosphoenol pyruvate carboxykinase, inhibitors of glycogen synthase kinase, inhibitors of pyruvate dehydrogenase kinase.

Modulators of glucose transporter-4, somatostatin receptor 3 agonists (e.g. MK-4256).

One or more lipid lowering agents are also suitable as combination partners, for example: 3-hydroxy-3-methylglutaryl-coenzym-A-reductase (HMG-CoA-reductase) inhibitors such as simvastatin (e.g. Zocor®, Inegy®, Simcor®), atorvastatin (e.g. Sortis®, Caduet®), rosuvastatin (e.g. Crestor®), pravastatin (e.g. Lipostat®, Selipran®), fluvastatin (e.g. Lescol®), pitavastatin (e.g. Livazo®, Livalo®), lovastatin (e.g. Mevacor®, Advicor®), mevastatin (e.g. Compactin®), rivastatin, cerivastatin (e.g. Lipobay®), fibrates such as bezafibrate (e.g. Cedur® retard), ciprofibrate (e.g. Hyperlipen®), fenofibrate (e.g. Antara®, Lipofen®, Lipanthyl®), gemfibrozil (e.g. Lopid®, Gevilon®), etofibrate, simfibrate, ronifibrate, clinofibrate, pemafibrate, clofibrate, clofibride, nicotinic acid and derivatives thereof (e.g. niacin, including slow release formulations of niacin), nicotinic acid receptor 1 agonists (e.g. GSK-256073), PPAR-delta agonists, acetyl-CoA-acetyltransferase (ACAT) inhibitors (e.g. avasimibe), cholesterol absorption inhibitors (e.g. ezetimibe, Ezetrol®, Zetia®, Liptruzet®, Vytorin®, S-556971), bile acid-binding substances (e.g. cholestyramine, colesevelam), ileal bile acid transport (IBAT) inhibitors (e.g. GSK-2330672, LUM-002), microsomal triglyceride transfer protein (MTP) inhibitors (e.g. Iomitapide (AEGR-733), SLx-4090, granotapide), modulators of proprotein convertase subtilisin/kexin type 9 (PCSK9) (e.g. alirocumab (e.g. Praluent®), evolocumab (e.g. Repatha®), LGT-209, PF-04950615, MPSK3169A, LY3015014, ALD-306, ALN-PCS, BMS-962476, SPC5001, ISIS-394814, 1B20, LGT-210, 1D05, BMS-PCSK9Rx-2, SX-PCK9, RG7652), LDL receptor up-regulators, for example liver selective thyroid hormone receptor beta agonists (e.g. eprotirome (KB-2115), MB07811, sobetirome (QRX-431), VIA-3196, ZYT1), HDL-raising compounds such as: cholesteryl ester transfer protein (CETP) inhibitors (e.g. anacetrapib (MK0859), dalcetrapib, evacetrapib, JTT-302, DRL-17822, TA-8995, R-1658, LY-2484595, DS-1442), or dual CETP/PCSK9 inhibitors (e.g. K-312), ATP-binding cassette (ABC1) regulators, lipid metabolism modulators (e.g. BMS-823778, TAP-301, DRL-21994, DRL-21995), phospholipase A2 (PLA2) inhibitors (e.g. darapladib, Tyrisa®, varespladib, rilapladib), ApoA-1 enhancers (e.g. RVX-208, CER-001, MDCO-216, CSL-112), cholesterol synthesis inhibitors (e.g. ETC-1002), lipid metabolism modulators (e.g. BMS-823778, TAP-301, DRL-21994, DRL-21995) and omega-3 fatty acids and derivatives thereof (e.g. icosapent ethyl (AMR101), Epanova®, Lovaza®, Vascepa®, AKR-063, NKPL-66, PRC-4016, CAT-2003).

HDL-raising compounds such as: CETP inhibitors (e.g. Torcetrapib, Anacetrapid, Dalcetrapid, Evacetrapid, JTT-302, DRL-17822, TA-8995) or ABC1 regulators.

Other suitable combination partners are one or more active substances for the treatment of obesity, such as for example:

Bromocriptine (e.g. Cycloset®, Parlodel®), phentermine and phentermine formulations or combinations (e.g. Adipex-P, lonamin, Qsymia®), benzphetamine (e.g. Didrex®), diethylpropion (e.g. Tenuate®), phendimetrazin (e.g. Adipost®, Bontril®), bupropion and combinations (e.g. Zyban®, Wellbutrin XL®, Contrave®, Empatic®), sibutramine (e.g. Reductil®, Meridia®), topiramat (e.g. Topamax®), zonisamid (e.g. Zonegran®), tesofensine, opioid antagonists such as naltrexone (e.g. Naltrexin®, naltrexone and bupropion), cannabinoid receptor 1 (CB1) antagonists (e.g. TM-38837), melanin-concentrating hormone (MCH-1) antagonists (e.g. BMS-830216, ALB-127158(a)), MC4 receptor agonists and partial agonists (e.g. AZD-2820, RM-493), neuropeptide Y5 (NPY5) or NPY2 antagonists (e.g. velneperit, S-234462), NPY4 agonists (e.g. PP-1420), beta-3-adrenergic receptor agonists, leptin or leptin mimetics, agonists of the 5-hydroxytryptamine 2c (5HT2c) receptor (e.g. lorcaserin, Belviq®), pramlintide/metreleptin, lipase inhibitors such as cetilistat (e.g. Cametor®), orlistat (e.g. Xenical®, Calobalin®), angiogenesis inhibitors (e.g. ALS-L1023), betahistidin and histamine H3 antagonists (e.g. HPP-404), AgRP (agouti related protein) inhibitors (e.g. TTP-435), serotonin re-uptake inhibitors such as fluoxetine (e.g. Fluctine®), duloxetine (e.g. Cymbalta®), dual or triple monoamine uptake inhibitors (dopamine, norepinephrine and serotonin re-uptake) such as sertraline (e.g. Zoloft®), tesofensine, methionine aminopeptidase 2 (MetAP2) inhibitors (e.g. beloranib), and antisense oligonucleotides against production of fibroblast growth factor receptor 4 (FGFR4) (e.g. ISIS-FGFR4Rx) or prohibitin targeting peptide-1 (e.g. Adipotide®).

Other suitable combination partners are one or more active substances for the treatment of fatty liver diseases including non-alcoholic fatty liver disease (NAFLD) and non-alcoholic steatohepatitis (NASH), such as for example:

Insulin sensitizers (e.g. rosiglitazone, pioglitazone), other PPAR modulators (e.g. elafibranor, saroglitazar, IVA-337), FXR agonists (e.g. obethicolic acid (INT-747), GS-9674, LJN-452, EDP-305), FGF19 analogues (e.g. NGM-282), FGF21 analogues (PF-05231023), GLP-1 analogues (e.g. Iiraglutide), SCD1 inhibitors (e.g. aramchol), anti-inflammatory compounds (e.g. CCR2/CCR5 antagonist cenicriviroc, pentamidine VLX-103), compounds reducing oxidative stress (e.g. ASK1 inhibitor GS-4997, VAP-1 inhibitor PXS-4728A), caspase inhibitors (e.g. emricasan), LOXL2 inhibitors (e.g. simtuzumab), galectin-3 protein inhibitors (e.g. GR-MD-02).

Moreover, combinations with drugs for influencing high blood pressure, chronic heart failure or atherosclerosis, for example: nitric oxide donors, AT1 antagonists or angiotensin II (AT2) receptor antagonists such as telmisartan (e.g. Kinzal®, Micardis®), candesartan (e.g. Atacand®, Blopress®), valsartan (e.g. Diovan®, Co-Diovan®), losartan (e.g. Cosaar®), eprosartan (e.g. Teveten®), irbesartan (e.g. Aprovel®, CoAprovel®), olmesartan (e.g. Votum®, Olmetec®), tasosartan, azilsartan (e.g. Edarbi®), dual angiotensin receptor blockers (dual ARBs), angiotensin converting enzyme (ACE) inhibitors, ACE-2 activators, renin inhibitors, prorenin inhibitors, endothelin converting enzyme (ECE) inhibitors, endothelin receptor (ET1/ETA) blockers, endothelin antagonists, diuretics, aldosterone antagonists, aldosterone synthase inhibitors, alpha-blockers, antagonists of the alpha-2 adrenergic receptor, beta-blockers, mixed alpha-/beta-blockers, calcium antagonists, calcium channel blockers (CCBs), nasal formulations of the calcium channel blocker diltiazem (e.g. CP-404), dual mineralocorticoid/CCBs, centrally acting antihypertensives, inhibitors of neutral endopeptidase, aminopeptidase-A inhibitors, vasopeptide inhibitors, dual vasopeptide inhibitors such as neprilysin-ACE inhibitors or neprilysin-ECE inhibitors, dual-acting AT receptor-neprilysin inhibitors, dual AT1/ETA antagonists, advanced glycation end-product (AGE) breakers, recombinant renalase, blood pressure vaccines such as anti-RAAS (renin-angiotensin-aldosteron-system) vaccines, AT1- or AT2-vaccines, drugs based on hypertension pharmacogenomics such as modulators of genetic polymorphisms with antihypertensive response, thrombocyte aggregation inhibitors, and others or combinations thereof are suitable.

In another aspect, this invention relates to the use of a compound according to the invention or a physiologically acceptable salt thereof combined with at least one of the active substances described above as a combination partner, for preparing a medicament which is suitable for the treatment or prevention of diseases or conditions which can be affected by binding to the GIP receptor and by modulating its activity. This is preferably a disease in the context of the metabolic syndrome, particularly one of the diseases or conditions listed above, most particularly diabetes or obesity or complications thereof.

The use of the compounds according to the invention, or a physiologically acceptable salt thereof, in combination with one or more active substances may take place simultaneously, separately or sequentially.

The use of the compound according to the invention, or a physiologically acceptable salt thereof, in combination with another active substance may take place simultaneously or at staggered times, but particularly within a short space of time. If they are administered simultaneously, the two active substances are given to the patient together.

Consequently, in another aspect, this invention relates to a medicament which comprises a compound according to the invention or a physiologically acceptable salt of such a compound and at least one of the active substances described above as combination partners, optionally together with one or more inert carriers and/or diluents.

The compound according to the invention, or physiologically acceptable salt or solvate thereof, and the additional active substance to be combined therewith may both be present together in one formulation, for example a tablet, capsule or solution, or separately in two identical or different formulations, for example as so-called kit-of-parts.

Another subject of the present invention are processes for the preparation of the compounds of formula I and their salts and solvates, by which the compounds are obtainable, and which are exemplified in the following.

### LEGENDS TO THE FIGURES

FIG. 1. SEQ ID NO: 9, Blood glucose excursion during an intraperitoneal (i.p.) glucose tolerance test (ipGTT) in C57Bl/6 mice.

FIG. 2. SEQ ID NO: 9, Area under curve (AUC) analysis on blood glucose excursion data in the time period from t=0 h (time point of i.p. glucose challenge) towards t=2 h (after i.p. glucose challenge) shown in FIG. 1.

FIG. 3. SEQ ID NO: 9, Delta blood glucose excursion (normalized to the time point just prior to the i.p. glucose challenge, t=0 h) during an intraperitoneal glucose tolerance test (ipGTT) in C57Bl/6 mice.

FIG. 4. SEQ ID NO: 9, Incremental area under curve (AUCᵢ) analysis on delta blood glucose excursion data in the time period from t=0 h (time point of i.p. glucose challenge) towards t=2 h (after i.p. glucose challenge) shown in FIG. 3.

FIG. 5. SEQ ID NO: 9, Mean±SD Plasma concentration values after the administration of 0.1 mg/kg i.v. or 0.1 mg/kg s.c. to the male cynomolgus monkey.

FIG. 6. SEQ ID NO: 9, Mean±SD Plasma concentration values after the administration of 0.05 mg/kg i.v. or 0.1 mg/kg s.c. to the female Gottingen minipig.

### METHODS

Abbreviations employed are as follows:
AA amino acid
AEEA (2-(2-aminoethoxy)ethoxy)acetyl
ACN acetonitrile
Aib alpha-amino-isobutyric acid, 2-methylalanine
AUC Area under the curve
cAMP cyclic adenosine monophosphate
Boc tert-butyloxycarbonyl
BOP (benzotriazol-1-yloxy)tris(dimethylamino)phosphonium hexafluorophosphate
BSA bovine serum albumin
BW body weight
tBu tertiary butyl
CV column volume
dAla d-Ala, D-Ala, D-alanine
Dab (S)-2,4-diaminobutyric acid
Dap (S)-2,3-diaminopropionic acid
DCM dichloromethane
Dde 1-(4,4-dimethyl-2,6-dioxocyclohexylidene)-ethyl
ivDde 1-(4,4-dimethyl-2,6-dioxocyclohexylidene)-3-methyl-butyl
DIC N,N′-diisopropylcarbodiimide
DIO diet-induced obese
DIPEA N,N-diisopropylethylamine
dl deciliter
DLS Dynamic light scattering
DMEM Dulbecco's modified Eagle's medium
DMF dimethyl formamide
DMS dimethylsulfide
DODT 3,6-dioxa-1,8-octanedithiol
DPBS Dulbecco's phosphate-buffered saline
EDT ethanedithiol
EDTA ethylenediaminetetraacetic acid
EGTA Ethylene glycol-bis(2-aminoethylether)-N,N,N′,N′-tetraacetic acid
eq equivalents
FA formic acid
FBS fetal bovine serum
FI fluorescence intensity
Fmoc fluorenylmethyloxycarbonyl
g gram
GIP glucose-dependent insulinotropic polypeptide
GIPR GIP receptor
GLP-1 glucagon-like peptide 1
GLP-1R GLP-1 receptor
gGlu gamma-glutamate (γE, γGlu)
h hour(s)
HATU O-(7-azabenzotriazol-1-yl)-N,N,N′,N′-tetramethyluronium hexafluorophosphate
HBSS Hanks' Balanced Salt Solution
HBTU 2-(1H-benzotriazol-1-yl)-1,1,3,3-tetramethyl-uronium hexafluorophosphate
HEPES 2-[4-(2-hydroxyethyl)piperazin-1-yl]ethanesulfonic acid
HOAt 1-hydroxy-7-azabenzotriazole
HOBt 1-hydroxybenzotriazole
HOSu N-hydroxysuccinimide
HPLC High Performance Liquid Chromatography
HSA human serum albumin
HTRF Homogenous Time Resolved Fluorescence
IBMX 3-isobutyl-1-methylxanthine
i.p. intraperitoneal
ipGTT intraperitoneal glucose tolerance test
i.v. intravenous
kg kilogram
l liter
LC/MS Liquid Chromatography/Mass Spectrometry
M molar
MBHA 4-methylbenzhydrylamine
min minute(s)
ml m illiliter
mm millimeter
μm micrometer
mM millimolar
mmol millimole(s)
Mmt monomethoxy-trityl
n.a. not available
n.d. not determined
nM nanomolar
nm nanometer
nmol nanomole(s)
μmol micromole(s)
NMP N-methyl pyrrolidone
Palm palmitoyl
Pbf 2,2,4,6,7-pentamethyldihydro-benzofuran-5-sulfonyl
PBS phosphate buffered saline
PEG polyethylene glycol
PK pharmacokinetic
pM picomolar
RCF relative centrifugal acceleration
Rₕ Stoke radius
RP-HPLC reversed-phase high performance liquid chromatography
rpm revolutions per minute
s.c. subcutaneous
SD standard deviation
sec second(s)
SEM standard error of the mean
Stea stearyl
TFA trifluoroacetic acid
TFE trifluorethanol
ThT Thioflavin T
TIS/TIPS triisopropylsilane
Trt trityl/triphenymethyl
TSTU N,N,N′,N′-tetramethyl-O—(N-succinimidyl)uronium tetrafluoroborate
UHPLC Ultra High Performance Liquid Chromatography/Ultra high pressure liquid chromatography
UV ultraviolet
v volume

General Synthesis of Peptidic Compounds

Materials

Different Rink-Amide resins (e.g. 4-(2′,4′-Dimethoxyphenyl-Fmoc-aminomethyl)-phenoxyacetamido-norleucylaminomethyl resin, Merck Biosciences; 4-[(2,4-Dimethoxyphenyl)(Fmoc-amino)methyl]phenoxy acetamido methyl resin, Agilent Technologies) were used for the synthesis of peptide amides with loadings in the range of 0.2-0.7 mmol/g. Alternatively, different preloaded Wang resins (e.g. ((S)-(9H-Fluoren-9-yl)methyl (1-(tert-butoxy)-3-oxopropan-2-yl)carbamate resin, Fmoc-Ser(tBu)-Wang resin, Bachem) were used for the synthesis of peptide acids with loadings in the range of 0.2-0.7 mmol/g.

Fmoc protected natural amino acids were purchased e.g. from Protein Technologies Inc., Senn Chemicals, Merck Biosciences, Novabiochem, Iris Biotech, Bachem, Chem-Impex International or MATRIX Innovation. The following standard amino acids were used throughout the syntheses: Fmoc-L-Ala-OH, Fmoc-Arg(Pbf)-OH, Fmoc-L-Asn(Trt)-OH, Fmoc-L-Asp(OtBu)-OH, Fmoc-L-Cys(Trt)-OH, Fmoc-L-Gln(Trt)-OH, Fmoc-L-Glu(OtBu)-OH, Fmoc-Gly-OH, Fmoc-L-His(Trt)-OH, Fmoc-L-Ile-OH, Fmoc-L-Leu-OH, Fmoc-L-Lys(Boc)-OH, Fmoc-L-Met-OH, Fmoc-L-Phe-OH, Fmoc-L-Pro-OH, Fmoc-L-Ser(tBu)-OH, Fmoc-L-Thr(tBu)-OH, Fmoc-L-Trp(Boc)-OH, Fmoc-L-Tyr(tBu)-OH, Fmoc-L-Val-OH.

In addition, the following special amino acids were purchased from the same suppliers as above: Fmoc-L-Lys(ivDde)-OH, Fmoc-L-Lys(Dde)-OH, Fmoc-L-Lys(Mmt)-OH, Fmoc-Aib-OH, Fmoc-N-Me-Gly-OH, Boc-N-Me-L-Tyr(tBu)-OH, and Boc-L-Tyr(tBu)-OH.

Furthermore, the building blocks N-alpha-(9-fluorenylmethyloxycarbonyl)-N-epsilon-(N-alpha′-palmitoyl-L-glutamic-acid alpha′-t-butyl ester)-L-lysine, (2S)-6-[[2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetyl]amino]-2-(9H-fluoren-9-ylmethoxycarbonyl-amino)hexanoic acid (Fmoc-L-Lys[{AEEA}2-gGlu(OtBu)-C18OtBu]-OH), Fmoc-AEEA-OH ([2-[2-(Fmoc-amino)ethoxy]ethoxy]acetic acid, CAS-No. 166108-71-0), Fmoc-AEEA-AEEA-OH ([2-(2-(Fmoc-amino)ethoxy)ethoxy]acetic acid, CAS-No. 560088-89-3), Fmoc-L-Ile-Aib-OH, and Boc-L-Tyr-Aib-OH can be applied. These building blocks were either acquired from commercial sources or synthesized separately, e.g. via stepwise synthesis or solid phase synthesis as described for example in CN104356224.

Furthermore, the side chain building blocks
HO-{AEEA}2-gGlu(OtBu)-C18OtBu (2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid; (S)-22-(tert-butoxycarbonyl)-10,19,24-trioxo-3,6,12,15-tetraoxa-9,18,23-triazahentetracontane-1,41-dioic acid; CAS-No. 1118767-16-0),
HO-{AEEA}2-gGlu(OtBu)-C20OtBu (2-[2-[2-[[2-[2-[2-[[(4S)-5-tert-butoxy-4-[(20-tert-butoxy-20-oxo-eicosanoyl)amino]-5-oxo-pentanoyl]amino]ethoxy]ethoxy]acetyl]amino]ethoxy]ethoxy]acetic acid; CAS-No. 1188328-37-1),
HO-{AEEA}2-{gGlu(OtBu)}2-C18OtBu (2-[2-[2-[[2-[2-[2-[[(4S)-5-tert-butoxy-4-[[(4S)-5-tert-butoxy-4-[(18-tert-butoxy-18-oxo-octadecanoyl)amino]-5-oxo-pentanoyl]amino]-5-oxo-pentanoyl]amino]ethoxy]ethoxy]acetyl]amino]ethoxy]ethoxy]acetic acid),
HO-{AEEA}2-{gGlu(OtBu)}2-C20OtBu (2-[2-[2-[[2-[2-[2-[[(4S)-5-tert-butoxy-4-[[(4S)-5-tert-butoxy-4-[(20-tert-butoxy-20-oxo-eicosanoyl)amino]-5-oxo-pentanoyl]amino]-5-oxo-pentanoyl]amino]ethoxy]ethoxy]acetyl]amino]ethoxy]ethoxy]acetic acid),
HO-{Gly}3-gGlu(OtBu)-C18OtBu (2-[[2-[[2-[[(4S)-5-tert-butoxy-4-[(18-tert-butoxy-18-oxo-octadecanoyl)amino]-5-oxo-pentanoyl]amino]acetyl]amino]acetyl]amino]acetic acid), and
HO—{N-MeGly}3-gGlu(OtBu)-C18OtBu (2-[[2-[[2-[[(4S)-5-tert-butoxy-4-[(18-tert-butoxy-18-oxo-octadecanoyl)amino]-5-oxo-pentanoyl]-methyl-amino]acetyl]-methyl-amino]acetyl]-methyl-amino]acetic acid)

have been applied. These building blocks were either acquired from commercial sources (e.g. Chengdu Pukang) or synthesized separately, e.g. via stepwise synthesis or solid phase synthesis as described analogously in WO09022006, WO09115469, or WO15028966.

The solid phase peptide syntheses were performed for example on a Prelude Peptide Synthesizer (Mesa Laboratories/Gyros Protein Technologies) or a similar automated synthesizer using standard Fmoc chemistry and HBTU/DIPEA or HATU/DIPEA activation. DMF was used as the solvent.

Deprotection: 20% piperidine/DMF for 2×2.5 min.

Washes: 7×DMF.

Coupling 2:5:10 200 mM AA/500 mM HBTU/2M DIPEA in DMF 2× for 20 min.

Washes: 5×DMF.

HBTU/DIPEA activiation was used for all standard couplings.

HATU/DIPEA activiation was used for the following couplings: Ile-Aib, Aib-Lys[{AEEA}2-gGlu(OtBu)-C18OtBu], Lys[{AEEA}2-gGlu(OtBu)-C18OtBu]-Asp, Gln-Aib, Leu-Leu. HATU couplings were left reacting in general 2× for 40 min, sometimes 2× for 1 h, and also up to 12 h.

In cases where a Lys-side-chain was modified, Fmoc-L-Lys(ivDde)-OH, Fmoc-L-Lys(Dde)-OH or Fmoc-L-Lys(Mmt)-OH was used in the corresponding position. After completion of the synthesis, the ivDde group was removed according to a modified literature procedure (S. R. Chhabra et al., Tetrahedron Lett., 1998, 39, 1603), using 4% hydrazine hydrate in DMF. The Mmt group was removed by repeated treatment with AcOH/TFE/DCM (1/2/7) for 15 min at RT, the resin then repeatedly washed with DCM, 5% DIPEA in DCM and 5% DIPEA in DCM/DMF. The following acylations were carried out by treating the resin with the N-hydroxy succinimide esters of the desired acid or using the free acids with coupling reagents like HBTU/DIPEA, HATU/DIPEA, HATU/HOAt/DIPEA or HOBt/DIC.

Stepwise attachment of acyl sidechains e.g. {AEEA}2-gGlu-C180H attachment to peptide:

The deprotection of the Mmt-group from the epsilon amino group of the lysine was carried out with 3×30 ml of a mixture of acetic acid and trifluoroethanol in dichloromethane (1:2:7) 15 min each. The resin was washed with DCM (3×), 5% DIPEA in DCM (3×), DCM (2×) and DMF (2×). The resin was then treated for 24 h with a solution of 2-[2-[2-(9H-fluoren-9-ylmethoxycarbonylamino)ethoxy]ethoxy]acetic acid (1 eq) in DMF preactivated with HATU (3 eq), HOAt (3 eq.), and DIPEA (4 eq). The product was washed with DMF, dichloromethane, ether and dried. After cleavage of the Fmoc protecting group with piperidine (20% in DMF), the procedure above was repeated to yield the 2-[2-[2-[[2-[2-[2-(9H-fluoren-9-ylmethoxycarbonylamino)ethoxy]ethoxy]acetyl]amino]ethoxy]ethoxy]acetic amide derivative. The Fmoc protecting group was cleaved and the resin was treated overnight with a solution of (4S)-5-tert-butoxy-4-(9H-fluoren-9-ylmethoxycarbonylamino)-5-oxo-pentanoic acid (1 eq) in DMF preactivated with HATU (3 eq), HOAt (3 eq.), and DIPEA (4 eq). The resin was washed as above. The Fmoc protecting group was cleaved and the product treated with a solution of 18-tert-butoxy-18-oxo-octadecanoic acid (1 eq) in DMF preactivated with HATU (3 eq), HOAt (3 eq), and DIPEA (4 eq). The resin was washed as above.

The peptides that have been synthesized on the automated synthesizer were cleaved from the resin with King's cleavage cocktail consisting of 82.5% TFA, 5% phenol, 5% water, 5% thioanisole, and 2.5% EDT or a modified cleavage cocktail consisting of 82.5% TFA, 5% phenol, 5% water, 5% thioanisole, and 2.5% DODT. The crude peptides were then precipitated in diethyl or diisopropyl ether, centrifuged, and lyophilized. Peptides were analyzed by analytical HPLC and checked by ESI mass spectrometry. Crude peptides were purified by a conventional preparative RP-HPLC purification procedure.

Alternatively, peptides were synthesized by a manual synthesis procedure.

Solid Phase Synthesis (Manual Synthesis Procedure)

0.3 g Desiccated Rink amide MBHA Resin (0.5-0.8 mmol/g) was placed in a polyethylene vessel equipped with a polypropylene filter. Resin was swollen in DCM (15 ml) for 1 h and DMF (15 ml) for 1 h. The Fmoc group on the resin was de-protected by treating it twice with 20% (v/v) piperidine/DMF solution for 5 and 15 min. The resin was washed with DMF/DCM/DMF (6/6/6 time each). A Kaiser test (quantitative method) was used for the confirmation of removal of Fmoc from solid support. The C-terminal Fmoc-amino acid (5 equiv. excess corresponding to resin loading) in dry DMF was added to the de-protected resin and coupling of the next Fmoc-amino acid was initiated with 5 equivalent excess of DIC and HOBT in DMF. The concentration of each reactant in the reaction mixture was approximately 0.4 M. The mixture was rotated on a rotor at room temperature for 2 h. Resin was filtered and washed with DMF/DCM/DMF (6/6/6 time each). Kaiser test on peptide resin aliquot upon completion of coupling was negative (no colour on the resin). After the first amino acid attachment, the unreacted amino group, if any, in the resin was capped used acetic anhydride/pyridine/DCM (1/8/8) for 20 min to avoid any deletion of the sequence. After capping, resin was washed with DCM/DMF/DCM/DMF (6/6/6/6 time each). The Fmoc group on the C-terminal amino acid attached peptidyl resin was deprotected by treating it twice with 20% (v/v) piperidine/DMF solution for 5 and 15 min. The resin was washed with DMF/DCM/DMF (6/6/6 time each). The Kaiser test on peptide resin aliquot upon completion of Fmoc-deprotection was positive.

The remaining amino acids in target sequence on Rink amide MBHA Resin were sequentially coupled using FmocAA/DIC/HOBt method using 5 equivalent excess corresponding to resin loading in DMF. The concentration of each reactant in the reaction mixture was approximately 0.4 M. The mixture was rotated on a rotor at room temperature for 2 h. Resin was filtered and washed with DMF/DCM/DMF (6/6/6 time each). After each coupling step and Fmoc deprotection step, a Kaiser test was carried out to confirm the completeness of the reaction.

After the completion of the linear sequence, the ε-amino group of lysine (protected with Dde) used as branching point or modification point was deprotected by using 2.5% hydrazine hydrate in DMF for 15 min×2 and washed with DMF/DCM/DMF (6/6/6 time each). The γ-carboxyl end of glutamic acid was attached to the ε-amino group of Lys using Fmoc-Glu(OH)-OtBu with DIC/HOBt method (5 equivalent excess with respect to resin loading) in DMF. The mixture was rotated on a rotor at room temperature for 2 h. The resin was filtered and washed with DMF/DCM/DMF (6/6/6 time each, 30 ml each). The Fmoc group on the glutamic acid was de-protected by treating it twice with 20% (v/v) piperidine/DMF solution for 5 and 15 min (25 ml each). The resin was washed with DMF/DCM/DMF (6/6/6 time each). A Kaiser test on peptide resin aliquot upon completion of Fmoc-deprotection was positive.

If the side chain branching also contains one more γ-glutamic acid, a second Fmoc-Glu(OH)-OtBu was used for the attachment to the free amino group of γ-glutamic acid with DIC/HOBt method (5 equivalent excess with respect to resin loading) in DMF. The mixture was rotated on a rotor at room temperature for 2 h. Resin was filtered and washed with DMF/DCM/DMF (6/6/6 time each, 30 ml each). The Fmoc group on the γ-glutamic acid was de-protected by treating it twice with 20% (v/v) piperidine/DMF solution for 5 and 15 min (25 ml). The resin was washed with DMF/DCM/DMF (6/6/6 time each). A Kaiser test on peptide resin aliquot upon completion of Fmoc-deprotection was positive.

### 18-[[(1 S)-1-carboxy-4-[2-[2-[2-[2-[2-(carboxymethoxy)ethoxy]ethylamino]-2-oxo-ethoxy]ethoxy]ethylamino]-4-oxo-butyl]amino]-18-oxo-octadecanoic acid attachment to peptide (Gradual Synthesis)

The deprotection of the Mmt-group from the epsilon amino group of the lysine was carried out with 3×30 ml acetic acid and trifluoroethanol in dichloromethane (1:2:7). The resin was then treated for 24 h with a solution of 2-[2-[2-(9H-fluoren-9-ylmethoxycarbonylamino)ethoxy]ethoxy]acetic acid (1 eq) in DMF preactivated with TSTU (3 eq), DIPEA (3 eq), and N-hydroxy-bezotriazole (3 eq). The product was washed with DMF, dichloromethane, ether and dried. After cleavage of the Fmoc protection group with piperidine (20% in DMF), the procedure above was repeated to yield 2-[2-[2-[[2-[2-[2-(9H-fluoren-9-ylmethoxycarbonylamino)ethoxy]ethoxy]acetyl]amino]ethoxy]ethoxy]acetic amide derivative. The Fmoc protecting group was cleaved and the resin was treated overnight with a solution of (4S)-5-tert-butoxy-4-(9H-fluoren-9-ylmethoxycarbonylamino)-5-oxo-pentanoic acid (1 eq) in DMF preactivated with TSTU (3 eq), DIPEA (3 eq), and N-hydroxy-bezotriazole (3 eq). The resin was washed as above. The Fmoc protection group was cleaved and the product treated with a solution of 18-tert-butoxy-18-oxo-octadecanoic acid (1 eq) in DMF preactivated with TSTU (3 eq), DIPEA (3 eq), and N-hydroxy-bezotriazole (3 eq). The t-butylester was cleaved in the final peptide cleavage from the resin.

Final Cleavage of Peptide from the Resin (Manual Synthesis Procedure)

The peptidyl resin synthesized by manual synthesis was washed with DCM (6×10 ml), MeOH (6×10 ml) and ether (6×10 ml) and dried in vacuum desiccators overnight. The cleavage of the peptide from the solid support was achieved by treating the peptide-resin with reagent cocktail (92% TFA, 2% thioanisole, 2% phenol, 2% water and 2% TIPS) at room temperature for 3 to 4 h. Cleavage mixture was collected by filtration and the resin was washed with TFA (2 ml) and DCM (2×5 ml). The excess TFA and DCM was concentrated to small volume under nitrogen and a small amount of DCM (5-10 ml) was added to the residue and evaporated under nitrogen. The process was repeated 3-4 times to remove most of the volatile impurities. The residue was cooled to 0° C. and anhydrous ether was added to precipitate the peptide. The precipitated peptide was centrifuged, the supernatant ether was removed, fresh ether was added to the peptide and re-centrifuged. The crude sample was purified by preparative HPLC and lyophilized. The identity of peptide was confirmed by LCMS.

In addition, a different route for the introduction of the lysine side chain is used, applying a prefunctionalized building block where the side chain is already attached to the lysine (e.g. Fmoc-L-Lys[{AEEA}2-gGlu(OtBu)-C18OtBu]-OH) as coupling partner in the peptide synthesis. 0.67 mmol of peptide resin bearing an amino-group is washed with 20 ml of dimethylformamide. 2.93 g of Fmoc-L-Lys[{AEEA}2-gGlu(OtBu)-C18OtBu]-OH is dissolved in 20 ml of dimethylformamide together with 310 mg of hydroxybenzotriazol hydrate and 0.32 ml of diisopropylcarbodiimide. After stirring of 5 min the solution is added to the resin. The resin is agitated for 20 h and then washed 3 times with 20 ml of dimethylformamide each. A small resin sample is taken and subjected to the Kaiser-test and the Chloranil-test (E. Kaiser, R. L. Colescott, C. D. Bossinger, P. I. Cook, Anal. Biochem. 1970, 34, 595-598; Chloranil-Test: T. Vojkovsky, Peptide Research 1995, 8, 236-237). This procedure avoids the need of a selective deprotection step as well as the selective attachment of the side chain building blocks on a very advanced synthesis intermediate.

Analytical HPLC/UHPLC

Method A: Detection at 214 nm
column: Waters ACQUITY UPLCO CSH™ C18 1.7 μm (150×2.1 mm) at 50° C.
solvent: H₂O+0.05% TFA:ACN+0.045% TFA (flow 0.5 ml/min)
gradient: 80:20 (0 min) to 80:20 (3 min) to 25:75 (23 min) to 5:95 (23.5 min) to 5:95 (26.5 min) to 80:20 (27 min) to 80:20 (33 min)

optionally with mass analyzer: LCT Premier, electrospray positive ion mode

Method B: Detection at 214 nm
column: Waters ACQUITY UPLCO CSH™ C18 1.7 μm (150×2.1 mm) at 50° C.
solvent: H₂O+0.05% TFA:ACN+0.035% TFA (flow 0.5 ml/min)
gradient: 80:20 (0 min) to 80:20 (3 min) to 25:75 (23 min) to 2:98 (23.5 min) to 2:98 (30.5 min) to 80:20 (31 min) to 80:20 (37 min)
mass analyzer: Agilent 6230 Accurate-Mass TOF or Agilent 6550 Funnel Q-TOF; both equipped with a Dual Agilent Jet Stream ESI ion source.

Method C: Detection at 214 nm
column: Waters ACQUITY UPLCO CSH™ C18 1.7 μm (150×2.1 mm) at 70° C.
solvent: H₂O+0.05% TFA:ACN+0.035% TFA (flow 0.5 ml/min)
gradient: 63:37 (0 min) to 63:37 (3 min) to 45:55 (23 min) to 2:98 (23.5 min) to 2:98 (30.5 min) to 63:37 (31 min) to 63:37 (38 min)
mass analyzer: Agilent 6230 Accurate-Mass TOF, Agilent Jet Stream ESI

General Preparative HPLC Purification Procedure

The crude peptides were purified either on an Äkta Purifier System, a Jasco semiprep HPLC System, an Agilent 1100 HPLC system or a similar HPLC system.

Preparative RP-C18-HPLC columns of different sizes and with different flow rates were used depending on the amount of crude peptide to be purified, e.g. the following columns have been used: Waters XSelect CSH C18 OBD Prep 5 μm 30×250 mm, Waters SunFire C18 OBD Prep 5 μm 30×250 mm, Waters SunFire C18 OBD Prep 5 μm 50×150 mm, and Phenomenex Luna Prep C18 5 μm 21.2×250 mm. Acetonitrile (B) and water+0.1% TFA (A) or water+0.1% FA (A) were employed as eluents. Product-containing fractions were collected and lyophilized to obtain the purified product, typically as TFA salt.

Alternatively, the peptides can be isolated as acetate salts via the following procedure: The peptide was dissolved in water and the solution adjusted to pH 7.05 with NaHCO₃. Then, the dissolved compound was purified with a RP Kinetex 21.2×250 mm (Column Volume CV 88 ml, 5 μm, C18, 100A, Åkta avant 25): The column was equilibrated with solvent A (3×CV), the compound was injected and then washed with a mixture of solvent A (95%) and solvent B (5%) with 3 CV. Then, a gradient solvent A:B (95:5) to A:B (20:80) was run with 15 CV. The purified peptide was collected and lyophilized.
column: Kinetex AXIA 5 μm C18 21.2×250 mm
solvent: A (H₂O+0.5% acetic acid): B (ACN+H₂O+0.5% acetic acid) (flow 7 ml/min)
gradient: 95:5 (0 min) to 95:5 (37 min) to 20:80 (180 min) to 0:100 (6 min)

Solubility Assessment

Prior to the solubility measurement of a peptide batch, its purity was determined through UHPLC/MS.

For solubility testing the target concentration was 10 mg pure compound/ml. Therefore, solutions from solid samples were prepared in a buffer system with a concentration of 10 mg/ml compound based on the previously determined % purity:

Solubility buffer system A) 100 mM phosphate buffer pH 7.4

Solubility buffer system B) 8 mM phosphate buffer pH 7.4, 14 mg/ml propylene glycol, 5.5 mg/ml phenol

Solubility buffer system C) 100 mM phosphate buffer pH 7.4, 2.7 mg/ml m-cresol

UHPLC-UV was performed after 1 h of gentle agitation and storage at 5° C. over night (24 h) from the supernatant, which was obtained after 15 min of centrifugation at 2500 RCF (relative centrifugal acceleration).

The solubility was determined by the comparison of the UV peak area of 2 μl-injection of a buffered sample diluted 1:10 with a standard curve of a reference peptide with known concentration. The different UV extinction coefficients of sample and reference peptide were calculated based on the different amino acid sequences and considered in the concentration calculation.

The analytical method used was Analytical UHPLC Method A.

Chemical Stability Assessment

Purity of a peptide batch was determined through UHPLC/MS prior to chemical stability measurement. The target concentration was 300 pM pure compound. Solutions from solid samples were prepared in the following buffer systems with a concentration of ˜300 μM compound based on the previously determined % purity:

Chemical stability buffer system A) 20 mM phosphate buffer pH 7.4,

Chemical stability buffer system B) 8 mM phosphate buffer pH 7.4, 14 mg/ml propylene glycol, 5.5 mg/ml phenol

Chemical stability buffer system C) 100 mM phosphate buffer pH 7.4, 2.7 mg/ml m-cresol

Prepared solutions were filtered through 0.22 μM pore size and filled into sterilized glass containers under laminar flow conditions.

Glass containers were stored for 28 days at 5 and 40° C. After this time, the samples were centrifuged for 15 min at 2500 RCF. Then 1.5 μl of the undiluted supernatant were analysed with UHPLC-UV.

The chemical stability was rated through the relative loss of purity calculated by the equation:

[(purity after 28 days at 5° C.)−(purity after 28 days at 40° C.)]/(purity after 28 days at 5° C.)]*100%

The purity is calculated as

[(peak area peptide)/(total peak area)]*100%

The analytical methods used were Analytical UHPLC Method B or C.

Dynamic Light Scattering (DLS) for the Assessment of Physical Stability

A monochromatic and coherent light beam (laser) is used to illuminate the liquid sample. Dynamic Light Scattering (DLS) measures light scattered from particles (1 nm≤radius≤1 μm) that undergo Brownian motion. This motion is induced by collisions between the particles and solvent molecules, that themselves are moving due to their thermal energy. The diffusional motion of the particles results in temporal fluctuations of the scattered light (R. Pecora, Dynamic Light Scattering: Applications of Photon Correlation Spectroscopy, Plenum Press, 1985).

The scattered light intensity fluctuations are recorded and transformed into an autocorrelation function. By fitting the autocorrelation curve to an exponential function, the diffusion coefficient D of the particles in solution can be derived. The diffusion coefficient is then used to calculate the hydrodynamic radius Rₕ (or apparent Stokes radius) through the Stokes-Einstein equation assuming spherical particles. This calculation is defined in ISO 13321 and ISO 22412 (International Standard ISO13321 Methods for Determination of Particle Size Distribution Part 8: Photon Correlation Spectroscopy, International Organisation for Standardisation (ISO) 1996; International Standard ISO22412 Particle Size Analysis—Dynamic Light Scattering, International Organisation for Standardisation, 2008).

In case of polydisperse samples, the autocorrelation function is the sum of the exponential decays corresponding to each of the species. The temporal fluctuations of the scattered light can then be used to determine the size distribution profile of the particle fraction or family. The first order result is an intensity distribution of scattered light as a function of the particle size. The intensity distribution is naturally weighted according to the scattering intensity of each particle fraction or family. For biological materials or polymers, the particle scattering intensity is proportional to the square of the molecular weight. Thus, small amount of aggregates/agglomerates or presence or a larger particle species can dominate the intensity distribution. However, this distribution can be used as a sensitive detector for the presence of large material in the sample.

The DLS technique produces distributions with inherent peak broadening. The polydispersity index % Pd is a measure of the width of the particle size distribution and is calculated by standard methods described in ISO13321 and ISO22412 [International Standard ISO13321 Methods for Determination of Particle Size Distribution Part 8: Photon Correlation Spectroscopy, International Organisation for Standardisation (ISO) 1996; International Standard ISO22412 Particle Size Analysis—Dynamic Light Scattering, International Organisation for Standardisation (ISO) 2008].

Solutions from solid samples were prepared in buffer systems (see below) with a target concentration of 300 μM compound based on the previously determined % purity.

DLS buffer system A) 20 mM phosphate buffer pH 7.4

DLS buffer system B) 8 mM phosphate buffer pH 7.4, 14 mg/ml propylene glycol, 5.5 mg/ml phenol

DLS buffer system C) 100 mM phosphate buffer pH 7.4, 2.7 mg/ml m-cresol Solutions were filtered through 0.22 μm pore size and filled into sterilized glass containers under laminar flow conditions. For every peptide solution, the apparent hydrodynamic radius (Rₕ), the corresponding Scattering Intensity (I), and the Mass Contribution (M) were determined as an average over 3-6 replicates from the intensity distribution of the scattered light averaging only high-quality measurements. Relative standard deviations (RSD) for these parameters were calculated from the same number of replicates.

DLS measurements were performed on a DynaPro Plate Reader II (Wyatt Technology, Santa Barbara, Calif., US) and using one of the following black, low volume, and non-treated plates: polystyrene 384-assay plate with clear bottom (Corning, N.Y., US), or cyclo olefin polymer (COP) 384-assay plate with clear bottom (Aurora, Mont., US), or polystyrene 384-assay plate with clear bottom (Greiner Bio-One, Germany). The data were processed with the Dynamics software provided by Wyatt Technology. Parameters of the particle size distribution were determined with non-negatively constrained least squares (NNLS) methods using DynaLS algorithms. Measurements were taken at 25° C. with an 830 nm laser light source at an angle of 1580.

ThT Assay for the Assessment of Physical Stability

Low physical stability of a peptide solution may lead to amyloid fibril formation, which is observed as well-ordered, thread-like macromolecular structures in the sample, which eventually may lead to gel formation. Thioflavin T (ThT) is widely used to visualize and quantify the presence of misfolded protein aggregates [Biancalana et al., Biochim. Biophys. Acta 2010, 1804(7), 1405]. When it binds to fibrils, such as those in amyloid aggregates, the dye displays a distinct fluorescence signature [Naiki et al., Anal. Biochem. 1989, 177, 244; LeVine et al., Methods. Enzymol. 1999, 309, 274]. The time course for fibril formation often follows the characteristic shape of a sigmoidal curve and can be separated into three regions: a lag phase, a fast growth phase, and a plateau phase.

The typical fibril formation process starts with the lag phase in which the amount of partially folded peptide turned into fibrils is not significant enough to be detected. The lag-time corresponds to the time the critical mass of the nucleus is built. Afterwards, a drastic elongation phase follows, and fibril concentration increases rapidly.

Investigations were carried out to determine fibrillation tendencies under stress conditions by shaking at 37° C. within Fluoroskan Ascent FL or Fluoroskan Ascent.

For the tests in Fluoroskan Ascent (FL), 200 μl sample were placed into a 96-well mictrotiter plate PS, flat bottom, Greiner Fluotrac No. 655076. Plates were sealed with Scotch Tape (Qiagen). Samples were stressed by continuous cycles of 10 sec shaking at 960 rpm and 50 sec rest period at 37° C. The kinetic was monitored by measuring fluorescence intensity every 20 min.

Peptides were diluted in a buffer system to a final concentration of 3 mg/ml. 20 μl of a 10.1 mM ThT solution in H₂O were added to 2 ml of peptide solution to receive a final concentration of 100 μM ThT. For each sample eight replicates were tested.

ThT buffer system A) 100 mM phosphate buffer pH 7.4

ThT buffer system B) 100 mM phosphate buffer pH 7.4, 2.7 mg/ml m-cresol

In Vitro Cellular Assays for GLP-1 and GIP Receptor Efficacy (HEK-293 Cell Line Over-Expressing Receptors)

Agonism of compounds at the human glucagon-like peptide-1 (GLP-1), or glucose-dependent insulinotropic polypeptide (GIP) receptors was determined by functional assays measuring cAMP response of recombinant PSC-HEK-293 cell lines stably expressing human GLP-1, or GIP receptors, respectively.

The cells were grown in a T-175 culture flask placed at 37° C. to near confluence in medium (DMEM/10% FBS) and collected in 2 ml vials in cell culture medium containing 10% DMSO in concentration of 10-50 million cells/ml. Each vial contained 1.8 ml cells. The vials were slowly frozen to −80° C. in isopropanol, and then transferred in liquid nitrogen for storage.

Prior to their use, frozen cells were thawed quickly at 37° C. and washed (5 min at 900 rpm) with 20 ml cell buffer (1× HBSS; 20 mM HEPES, plus 0.1% HSA if indicated in Example conditions). Cells were resuspended in assay buffer (cell buffer plus 2 mM IBMX) and adjusted to a cell density of 1 million cells/ml.

For measurement of cAMP generation, 5 μl cells (final 5000 cells/well) and 5 μl of test compound were added to a 384-well plate, followed by incubation for 30 min at room temperature.

The cAMP generated was determined using a kit from Cisbio Corp. based on HTRF (Homogenous Time Resolved Fluorescence). The cAMP assay was performed according to manufacturer's instructions (Cisbio).

After addition of HTRF reagents diluted in lysis buffer (kit components), the plates were incubated for 1 h, followed by measurement of the fluorescence ratio at 665/620 nm. In vitro potency of agonists was quantified by determining the concentrations that caused 50% activation of the maximal response (EC50).

In Vitro Cellular Assay for GIP Receptor Efficacy (Adipocytes)

Additionally, GIPR agonism of compounds was determined by a functional assay measuring cAMP response of human adipocytes endogenously expressing the human GIP receptor.

For this, one vial of human preadipocytes (˜10⁶ cells; Lonza) was thawed in a T-75 cell culture dish. The cells were cultivated at 37° C., 5% C02, 95% humidity in Preadipocyte Growth Medium with Supplement Mix from Promo Cell.

After 3 days, the cells were washed with PBS and 1.5 ml Trypsin, incubated for 4 min, then resuspended in medium, centrifugated for 10 min @ 300 rcf RT, resuspended again and distributed to four T-75 cell culture dishes. Again, the cells were cultivated at 37° C., 5% C02, 95% humidity.

After 5 days, the cells were washed with PBS and 1.5 ml Trypsin, incubated for 4 min, then resuspended in medium, centrifugated for 10 min @ 300 rcf RT, resuspended again and sawn in T-75 dishes (2.5×10⁶ cells per dish) in 15 ml Differentiation medium each.

The differentiation medium had the following composition: DMEM (Gibco), Ham's F10 (Gibco), 15 mM HEPES (Gibco), 3% FCS (PAA), 33 μM biotin (Sigma-Aldrich), 17 μM Pantothenate (Sigma-Aldrich), 0.1 μM human insulin (Sigma-Aldrich), 1 μM dexamethason (Sigma-Aldrich), 0.1 μM PPARgamma agonist (#R2408, Sigma-Aldrich), 0.6× Anti-Anti (#15240, ThermoFisher), 200 μM IBMX (AppliChem), and 0.01 μM L-thyroxine (Sigma-Aldrich).

After 6 days of differentiation, 5000 cells per well were dispensed on a 96-well plate (#CLS3694 from Corning®, Sigma-Aldrich). For measurement of cAMP generation, 25 μL of test compound was added to each well of the 96-well plate, followed by incubation for 30 min at room temperature.

The cAMP generated following test compound stimulation was determined using a kit from Cisbio Corp. based on HTRF (Homogenous Time Resolved Fluorescence). The cAMP assay was performed according to manufacturer's instructions (Cisbio).

The cAMP content of cells was determined using a kit from Cisbio Corp. based on HTRF (Homogenous Time Resolved Fluorescence).

After addition of HTRF reagents diluted in lysis buffer (kit components), the plates were incubated for 1 h, followed by measurement of the fluorescence ratio at 665/620 nm.

In vitro potency of agonists was quantified by determining the concentrations that caused 50% activation of the maximal response (EC50).

In Vitro Assays for Binding to the Human GIP and the Human GLP-1 Receptor

(1) Preparation of Membranes from HEK-293 Cells Over-Expressing GIPR or GLP-1R

HEK-293 cells recombinantly over-expressing GIPR or GLP-1R were grown to 50% confluency, washed with warm 1×PBS (Gibco) and detached in HEPES/EDTA-buffer (100 mM HEPES pH 7.5, 5 mM EDTA). Cells were harvested by centrifugation at 4° C. and 3000×g and the pellets were stored at −80° C. until further processing.

After thawing on ice, pellets were resuspended in HEPES/EDTA-buffer and homogenized on ice for 1 min using Ultra-Turray T25. After subsequent sonification the cell debris was removed by centrifugation at 1000×g and 4° C. Supernatants were then ultra-centrifuged at 100000×g and 4° C. under vacuum for 30 min. Pellets were resuspended in HEPES/EDTA/NaCl-buffer (20 mM HEPES, 1 mM EDTA, 150 mM NaCl; add 1 Complete Mini Protease inhibitor cocktail to 10 ml buffer) and protein content was determined via BCA-Protein assay.

(2) Measurement of Binding Activity of Test Compounds to Human GIPR or GLP-1R

For the measurement of the binding activity to GIPR or GLP-1R, [¹²⁵I]GIP or [¹²⁵I]GLP-1 (PerkinElmer), respectively, in a final concentration of 100 pM and a test compound in 10 concentrations were mixed with PVT-WGA SPA beads (0.125 mg/well; Perkin-Elmer) coated with HEK-293 cell membranes (1 μg/well of protein) expressing GLP-1R or GIPR in assay buffer [50 mM HEPES (pH 7.4, WAKO), 5 mM EGTA (WAKO), 5 mM MgCl₂ (WAKO), and 0.005% Tween 20 (BioRad)] and incubated at room temperature for 2 h. Specific binding was calculated as the difference between the amount of [¹²⁵I]labeled hot ligand (GIP, GLP-1) bound in the absence (total binding) and presence (nonspecific binding) of 1 and 2 μM unlabeled cold reference ligand, respectively.

Pharmacokinetic Evaluation of Exendin-4 Derivatives in Mice, Rats, Monkeys and Pigs

Compounds were administered in a suitable buffer system, e.g. PBS buffer solution at pH7.4 or DPBS solution at concentrations of 0.05, 0.1, 0.5 or 1 mg/ml depending on dose, species and administration volume.

Mice

Female C57Bl/6 mice were dosed 0.25 mg/kg, 0.5 mg/kg or 1 mg/kg intravenously (i.v.) or subcutaneously (s.c.). The mice were sacrificed, and blood samples were collected after 0.08, 0.25, 0.5, 1, 2, 4, 8, 24, 32, and 48 h post i.v. application and 0.25, 0.5, 1, 2, 4, 8, 24, 32, and 48 h post s.c. application, respectively. Plasma samples were analyzed after protein precipitation via liquid chromatography mass spectrometry (LC/MS). PK parameters and half-life were calculated using Phoenix-WinNonlin 8.1 using a non-compartmental model and linear trapezoidal interpolation calculation.

Rats

Male SD rats were dosed 0.25 mg/kg, 0.5 mg/kg or 1 mg/kg intravenously (i.v.) or subcutaneously (s.c.). Blood samples were collected after 0.08, 0.25, 0.5, 1, 2, 4, 8, 24, 32, and 48 h post i.v. application and 0.25, 0.5, 1, 2, 4, 8, 24, 32, and 48 h post s.c. application, respectively. Plasma samples were analyzed after protein precipitation via liquid chromatography mass spectrometry (LC/MS). PK parameters and half-life were calculated using Phoenix-WinNonlin 8.1 using a non-compartmental model and linear trapezoidal interpolation calculation.

Monkeys

Male cynomolgus monkeys were dosed 0.1 mg/kg intravenously (i.v.) or subcutaneously (s.c.). Blood samples were collected after 0.083, 0.25, 0.5, 1, 2, 4, 8, 24, 32, 48, and 72 h post i.v. application and 0.5, 1, 2, 4, 8, 24, 48, 72, and 96 h post s.c. application, respectively. Plasma samples were analyzed after protein precipitation via liquid chromatography mass spectrometry (LC/MS). PK parameters and half-life were calculated using Phoenix-WinNonlin 8.1 using a non-compartmental model and linear trapezoidal interpolation calculation.

Minipigs

Female Gottingen minipigs were dosed 0.05 mg/kg intravenously (i.v.) or 0.1 mg/kg subcutaneously (s.c.). Blood samples were collected at 0 h and after 0.083, 0.25, 0.5, 1, 2, 4, 8, 24, 32, 48, 56, 72, 80, and 96 h post i.v. application and at 0 h and after 0.25, 0.5, 1, 2, 4, 8, 24, 32, 48, 56, 72, 80, and 96 h post s.c. application, respectively. Plasma samples were analyzed after protein precipitation via liquid chromatography mass spectrometry (LC/MS). PK parameters and half-life were calculated using Phoenix-WinNonlin 8.1 using a non-compartmental model and linear trapezoidal interpolation calculation.

Acute Effects after Subcutaneous (s.c.) Treatment on Blood Glucose in an Intraperitoneal (i.p.) Glucose Tolerance Test (ipGTT) in Healthy, Male C57Bl/6 Mice

Healthy, normoglycemic male C57Bl/6NCrl mice were ordered in an age of 9-10 weeks with an approximate body weight (BW) of 24-26 g at Charles River Laboratories Deutschland GmbH, 97633 Sulzfeld, Germany. Mice were shipped grouped housed (N=4 per cage), acclimatized for one week and remained grouped housed throughout the entire study. Mice were housed under vivarium conditions that included a 12 h light/dark cycle (light phase 06:00 AM-06:00 PM), mean room temperature of 22±2° C. and a relative mean humidity of 55±10%. All animals had free access to food (Ssniff R/M-H diet) and water prior to study start. At study start mice were between 10-11 weeks old and had a mean BW of 24.7±0.13 g (n=48).

The primary objective of the study was to investigate the compound-induced lowering of the blood glucose excursion and improvement of glucose tolerance in a mouse ipGTT setting and therefore the primary parameters included blood glucose, delta blood glucose (normalized to the time point just prior to the i.p. glucose challenge, t=0 h) and their respective calculated area under curve (AUC) values. The study was performed as an acute, single dosing study with 6 groups and the male animals were randomly divided to groups of 7-8 mice per group. Dose-dependent pharmacodynamic blood glucose lowering efficacy of the GIPR agonist was analyzed with s.c. injections 6 h before the i.p. glucose load in the dose range from 3 up to 100 nmol/kg and compared to the vehicle group as well as the semaglutide positive control at a dose of 10 nmol/kg.

In more detail, mice were fed overnight and on the following morning brought to the lab with food removed but ad libitum access to water. Blood samplings (5 μl) were performed at: −6.5, −0.5, 0, 0.17, 0.5, 1, 1.5, 2 and 3 h before and after the glucose bolus of 1 g glucose per kg BW i.p. given at time point t=0 h. On time point t=0.17 h an additional K-EDTA plasma sample from 60-80 μl blood was taken for plasma insulin analysis. Blood was withdrawn from the tip of the tail. The GIPR agonist and semaglutide were dissolved in 10 mM phosphate buffer pH 7.4 with 2.3% glycerol and 0.01% polysorbat 20 (vehicle) and s.c. treatment was performed −6 h before the glucose load (t=0 h) using an injection volume of 5 ml/kg. The injection solutions were freshly prepared prior to the experiment using sterile filtered vehicle solution.

Blood glucose was determined enzymatically (Gluco-quant® Glucose/HK kit on Roche/Hitachi 912). Plasma insulin was determined using a mouse/rat insulin sandwich immunoassay kit from Meso Scale Discovery.

Data Collection and Statistical Analysis

All data were collected using Microsoft Excel. None of the data were collected online. The results are expressed as means±standard error of the mean (SEM). As primary study parameters, blood glucose, baseline (time point t=0 h) subtracted delta blood glucose and their respective calculated area under curve (AUC) values were determined. The respective AUC data were calculated using the trapezoid rule for the time period t=0 h until t=2 h.

The statistical analysis of the ipGTT blood glucose excursion data response following subcutaneous compound or vehicle treatment was performed on the calculated AUC values of blood glucose raw data as well as the calculated AUC values for baseline subtracted delta blood glucose data. In a first step the Levene's test was used to test for equality of variances between groups. Where Levene's test was significant (p s 0.05), a rank transformation of the calculated AUC data was applied to stabilize the variances before ANOVA analysis was conducted. Where Levene's test was not significant (p>0.05) ANOVA was conducted without prior rank transformation. In a second step a One-way ANOVA analysis for factor treatment followed by Dunnett's multiple comparisons test vs. vehicle group was used to test for statistical differences. Different levels of significance were defined as *=p<0.05, **=p<0.001, ***=p<0.0001. All analyses were performed using SAS (version 9.4) under Linux via the interface software EverSt@t V6.1.

### EXAMPLES

The invention is further illustrated by the following examples.

### Example 1: Synthesis of SEQ ID NO: 8

The solid phase synthesis as described in Methods was carried out on Novabiochem Rink-Amide resin (4-(2′,4′-Dimethoxyphenyl-Fmoc-aminomethyl)-phenoxyacetamido-norleucylaminomethyl resin), 100-200 mesh, loading of 0.35 mmol/g. The automated Fmoc-synthesis strategy was applied with HBTU/DIPEA-activation or HATU/DIPEA-activation depending on the amino acid sequence. In position 14 Fmoc-Lys(Mmt)-OH and in position 1 Boc-N-Me-L-Tyr(tBu)-OH were used in the solid phase synthesis protocol. The Mmt-group was cleaved from the peptide on resin as described in the Methods. Hereafter HO-{AEEA}2-gGlu(OtBu)-C18OtBu (CAS-No. 1118767-16-0) was coupled to the liberated amino-group employing DIPEA as base and HATU/HOAt as coupling reagents. The peptide was cleaved from the resin with King's cocktail (D. S. King, C. G. Fields, G. B. Fields, Int. J. Peptide Protein Res. 1990, 36, 255-266). The crude product was purified via preparative HPLC on a Waters column (Waters SunFire C18 OBD Prep 5 μm 50×150 mm) using an acetonitrile/water gradient (water with 0.1% TFA). The purified peptide was analysed by LCMS (Method B). Deconvolution of the mass signals found under the peak with retention time 14.86 min revealed the peptide mass 4968.61 which is in line with the expected value of 4968.60.

### Example 2: Synthesis of SEQ ID NO: 9

The solid phase synthesis as described in Methods was carried out on Novabiochem Rink-Amide resin (4-(2′,4′-Dimethoxyphenyl-Fmoc-aminomethyl)-phenoxyacetamido-norleucylaminomethyl resin), 100-200 mesh, loading of 0.35 mmol/g. The automated Fmoc-synthesis strategy was applied with HBTU/DIPEA-activation or HATU/DIPEA-activation depending on the amino acid sequence. In position 14 Fmoc-Lys(Mmt)-OH and in position 1 Boc-Tyr(tBu)-OH were used in the solid phase synthesis protocol. The Mmt-group was cleaved from the peptide on resin as described in the Methods. Hereafter, HO-{AEEA}2-gGlu(OtBu)-C18OtBu (CAS-No. 1118767-16-0) was coupled to the liberated amino-group employing DIPEA as base and HATU/HOAt as coupling reagents. The peptide was cleaved from the resin with King's cocktail (D. S. King, C. G. Fields, G. B. Fields, Int. J. Peptide Protein Res. 1990, 36, 255-266). The crude product was purified via preparative HPLC first on a Waters column (Waters SunFire C18 OBD Prep 5 μm 50×150 mm) using an acetonitrile/water gradient (water with 0.1% TFA) and thereafter via preparative HPLC on a Waters column (Waters Xselect CSH Prep C18 5 μm 50×150 mm) using an acetonitrile/water gradient (water with 0.1% TFA). The purified peptide was collected and lyophilized.

Afterwards, the peptide was dissolved in water, the pH adjusted to pH 7.05 with NaHCO₃ and purified for a third time via preparative HPLC (Åkta avant 25a, Column: RP Kinetex 21.2×250 mm, volume CV 88 ml, 5 μm, C18, 100A) using an acetonitrile/water gradient (both buffers with 0.5% acetic acid). The purified peptide was collected and lyophilized.

The purified peptide was analysed by LCMS (Method B). Deconvolution of the mass signals found under the peak with retention time 14.20 min revealed the peptide mass 4998.60 which is in line with the expected value of 4998.58.

### Example 3: Synthesis of SEQ ID NO: 11

The solid phase synthesis as described in Methods was carried out on Fmoc-Ser(tBu)-Wang resin ((S)-(9H-Fluoren-9-yl)methyl (1-(tert-butoxy)-3-oxopropan-2-yl)carbamate resin), 100-200 mesh, loading of 0.42 mmol/g. The automated Fmoc-synthesis strategy was applied with HBTU/DIPEA-activation or HATU/DIPEA-activation depending on the amino acid sequence. In position 14 Fmoc-Lys(Mmt)-OH and in position 1 Boc-Tyr(tBu)-OH were used in the solid phase synthesis protocol. The Mmt-group was cleaved from the peptide on resin as described in the Methods. Hereafter, HO-{AEEA}2-gGlu(OtBu)-C18OtBu (CAS-No. 1118767-16-0) was coupled to the liberated amino-group employing DIPEA as base and HATU/HOAt as coupling reagents. The peptide was cleaved from the resin with King's cocktail (D. S. King, C. G. Fields, G. B. Fields, Int. J. Peptide Protein Res. 1990, 36, 255-266). The crude product was purified via preparative HPLC first on a Waters column (Waters Xselect CSH Prep C18 5 μm 30×250 mm) using an acetonitrile/water gradient (water with 0.1% TFA) and thereafter via preparative HPLC on a Waters column (Waters Xselect CSH Prep C18 5 μm 50×150 mm) using an acetonitrile/water gradient (water with 0.1% formic acid). The purified peptide was collected and lyophilized.

The purified peptide was analysed by LCMS (Method B). Deconvolution of the mass signals found under the peak with retention time 14.21 min revealed the peptide mass 4999.58 which is in line with the expected value of 4999.56.

### Example 4: Synthesis of SEQ ID NO: 12

The solid phase synthesis as described in Methods was carried out on Novabiochem Rink-Amide resin (4-(2′,4′-Dimethoxyphenyl-Fmoc-aminomethyl)-phenoxyacetamido-norleucylaminomethyl resin), 100-200 mesh, loading of 0.35 mmol/g. The automated Fmoc-synthesis strategy was applied with HBTU/DIPEA-activation or HATU/DIPEA-activation depending on the amino acid sequence. In position 14 Fmoc-Lys(Mmt)-OH and in position 1 Boc-Tyr(tBu)-OH were used in the solid phase synthesis protocol. The Mmt-group was cleaved from the peptide on resin as described in the Methods. Hereafter, 8-(9-Fluorenylmethyloxycarbonyl-amino)-3,6-dioxaoctanoic acid (CAS-No. 166108-71-0) was coupled to the liberated amino-group employing DIPEA as base and HATU/HOAt as coupling reagents. The Fmoc-protecting group was deprotected using standard conditions and HO-{AEEA}2-gGlu(OtBu)-C18OtBu (CAS-No. 1118767-16-0) was coupled to the liberated amino-group employing DIPEA as base and HATU/HOAt as coupling reagents. The peptide was cleaved from the resin with King's cocktail (D. S. King, C. G. Fields, G. B. Fields, Int. J. Peptide Protein Res. 1990, 36, 255-266). The crude product was purified via preparative HPLC on a Waters column (Waters Xselect CSH Prep C18 5 μm 30×250 mm) using an acetonitrile/water gradient (water with 0.1% TFA). The purified peptide was collected and lyophilized.

The purified peptide was analysed by LCMS (Method B). Deconvolution of the mass signals found under the peak with retention time 12.24 min revealed the peptide mass 5143.68 which is in line with the expected value of 5143.65.

### Example 5: Synthesis of SEQ ID NO: 13

The solid phase synthesis as described in Methods was carried out on Novabiochem Rink-Amide resin (4-(2′,4′-Dimethoxyphenyl-Fmoc-aminomethyl)-phenoxyacetamido-norleucylaminomethyl resin), 100-200 mesh, loading of 0.35 mmol/g. The automated Fmoc-synthesis strategy was applied with HBTU/DIPEA-activation or HATU/DIPEA-activation depending on the amino acid sequence. In position 14 Fmoc-L-Lys[{AEEA}2-gGlu(OtBu)-C18OtBu] and in position 1 Boc-N-Me-L-Tyr(tBu)-OH were used in the solid phase synthesis protocol. The peptide was cleaved from the resin with King's cocktail (D. S. King, C. G. Fields, G. B. Fields, Int. J. Peptide Protein Res. 1990, 36, 255-266). The crude product was purified via preparative HPLC first on a Waters column (Waters SunFire C18 OBD Prep 5 μm 50×150 mm) using an acetonitrile/water gradient (water with 0.1% TFA) and thereafter via preparative HPLC on a Waters column (Waters Xselect CSH Prep C18 5 μm 30×250 mm) using an acetonitrile/water gradient (water with 0.1% formic acid). The purified peptide was collected and lyophilized. The purified peptide was analysed by LCMS (Method B). Deconvolution of the mass signals found under the peak with retention time 12.30 min revealed the peptide mass 5012.62 which is in line with the expected value of 5012.60.

In an analogous way, the other peptides listed in Table 2 were synthesized and characterized.

TABLE 2
list of synthesized peptides and comparison
of calculated vs. found molecular weight

| SEQ | | | | | |
|---|---|---|---|---|---|
| ID | calc. Mass | found | Monoisotopic or | Retention | |
| NO | monoisotopic | mass | average mass | time [min] | Method |
| 4 | 4954.59 | 4954.59 | monoisotopic | 13.09 | B |
| 5 | 4835.51 | 4835.52 | monoisotopic | 14.77 | B |
| 6 | 4877.55 | 4877.57 | monoisotopic | 14.98 | B |
| 7 | 4982.62 | 4982.65 | monoisotopic | 15.65 | B |
| 8 | 4968.60 | 4968.61 | monoisotopic | 14.86 | B |
| 9 | 4998.58 | 4998.60 | monoisotopic | 14.20 | B |
| 10 | 5026.61 | 5026.64 | monoisotopic | 15.0 | B |
| 11 | 4999.56 | 4999.58 | monoisotopic | 14.21 | B |
| 12 | 5143.65 | 5143.68 | monoisotopic | 12.24 | B |
| 13 | 5012.60 | 5012.62 | monoisotopic | 12.30 | B |
| 14 | 5127.62 | 5127.65 | monoisotopic | 12.14 | B |
| 15 | 5155.65 | 5155.68 | monoisotopic | 12.89 | B |
| 16 | 4879.50 | 4879.52 | monoisotopic | 13.93 | B |
| 17 | 4921.54 | 4921.56 | monoisotopic | 14.14 | B |
| 18 | 5083.63 | 5083.66 | monoisotopic | 14.55 | B |
| 19 | 5111.66 | 5111.69 | monoisotopic | 15.27 | B |
| 20 | 4955.57 | 4955.61 | monoisotopic | 14.85 | B |

### Example 6: Chemical Stability

Peptide samples were prepared in Chemical stability buffer system A or B and stability was assessed as described in Methods. The results are given in Table 3 and Table 4.

TABLE 3
Stability in Chemical stability buffer system A

| | Relative purity loss over |
|---|---|
| SEQ ID NO | 28 days at 40° C. [%] |
| 4 | 6.4 |
| 5 | 5.8 |
| 6 | 5.4 |
| 7 | 4.5 |
| 8 | 4.7 |
| 9 | 6.9 |
| 10 | 3.3 |
| 11 | 6.1 |
| 12 | n.d. |
| 13 | n.d. |
| 14 | n.d. |
| 15 | n.d. |
| 16 | n.d. |
| 17 | n.d. |

TABLE 4
Stability in Chemical stability buffer system B

| | Relative purity loss over |
|---|---|
| SEQ ID NO | 28 days at 40° C. [%] |
| 9 | 8.2 |

### Example 7: Solubility

Peptide samples were prepared in solubility buffer system A or C and solubility was assessed as described in Methods. The results are given in Table 5 and Table 6.

TABLE 5
Solubility in Solubility buffer system A

| SEQ ID | Soluble amount |
|---|---|
| NO | [mg/ml] |
| 4 | >9.5 |
| 5 | >9.3 |
| 6 | >10.8 |
| 7 | >6.9 |
| 8 | >7.5 |
| 9 | >30.0 |
| 10 | >8.0 |
| 11 | >9.8 |
| 12 | >8.8 |
| 13 | >10.2 |
| 14 | >9.3 |
| 15 | >10.3 |
| 16 | >10.0 |
| 17 | >10.9 |
| 18 | n.d. |
| 19 | n.d. |
| 20 | n.d. |

TABLE 6
Solubility in Solubility buffer system C

| SEQ ID | Soluble amount |
|---|---|
| NO | [mg/ml] |
| 4 | >8.4 |
| 5 | >8.3 |
| 6 | >8.9 |
| 7 | >5.9 |
| 8 | >6.4 |
| 9 | >9.3 |
| 10 | >8.1 |
| 11 | >9.8 |
| 12 | >9.1 |
| 13 | >11.0 |
| 14 | >9.7 |
| 15 | >9.2 |
| 16 | >9.9 |
| 17 | >11.8 |
| 18 | n.d. |
| 19 | n.d. |
| 20 | n.d. |

### Example 8: Stability as Assessed in the ThT Assay

Lag time in hours (h) in Thioflavin T (ThT) assay of peptide samples was determined in ThT buffer system A as described in Methods. The results are given in Table 7.

TABLE 7
Increase in fluorescence intensity (FI) and lag time in hours (h)
in Thioflavin T (ThT) assay for samples in ThT buffer system A

| SEQ ID | Increase | Lag time before |
|---|---|---|
| NO | in FI | increase [h] |
| 4 | no | >45 |
| 5 | no | >45 |
| 6 | no | >45 |
| 7 | n.d. | n.d. |
| 8 | no | >45 |
| 9 | no | >45 |
| 10 | n.d. | n.d. |
| 11 | n.d. | n.d. |
| 12 | n.d. | n.d. |
| 13 | n.d. | n.d. |
| 14 | n.d. | n.d. |
| 15 | n.d. | n.d. |
| 16 | n.d. | n.d. |
| 17 | n.d. | n.d. |
| 18 | n.d. | n.d. |
| 19 | n.d. | n.d. |
| 20 | n.d. | n.d. |

### Example 9: Stability as Assessed in the DLS Assay

The apparent hydrodynamic radius (Rₕ), the Scattering Intensity and Mass Contribution were determined after manufacturing (0 weeks) and after 4 weeks storage at 40° C. as described in Methods in DLS buffer system A or B. The results are given in Table 8 and Table 9.

TABLE 8
Apparent hydrodynamic radius (Rₕ) and Scattering Intensity
(I) and the Mass Contribution (M) as determined with
DLS for samples prepared in DLS buffer system A.

| | apparent Rₕ and SD | | I and SD | | M and SD | |
|---|---|---|---|---|---|---|
| SEQ | [nm] | | [%] | | [%] | |
| ID NO | 0 weeks | 4 weeks | 0 weeks | 4 weeks | 0 weeks | 4 weeks |
| 4 | 1.91 ± 0.05 | 1.9 ± 0.04 | 93 ± 1 | 93 ± 1 | 100 ± 0 | 100 ± 0 |
| 5 | 2.05 ± 0.04 | 1.99 ± 0.03 | 76 ± 2 | 89 ± 3 | 100 ± 0 | 100 ± 0 |
| 6 | 1.92 ± 0.03 | 1.94 ± 0.03 | 86 ± 2 | 83 ± 1 | 100 ± 0 | 100 ± 0 |
| 7 | 2.06 ± 0.03 | 2.04 ± 0.02 | 85 ± 2 | 73 ± 4 | 100 ± 0 | 100 ± 0 |
| 8 | 2.06 ± 0.03 | 2.00 ± 0.07 | 73 ± 3 | 78 ± 9 | 100 ± 0 | 100 ± 0 |
| 9 | 1.86 ± 0.03 | 1.87 ± 0.03 | 80 ± 3 | 80 ± 3 | 100 ± 0 | 100 ± 0 |
| 10 | 1.97 ± 0.03 | 1.91 ± 0.04 | 60 ± 3 | 75 ± 3 | 100 ± 0 | 100 ± 0 |
| 11 | 1.82 ± 0.04 | 1.83 ± 0.05 | 96 ± 1 | 85 ± 2 | 100 ± 0 | 100 ± 0 |
| 12 | n.d. | n.d. | n.d. | n.d. | n.d. | n.d. |
| 13 | n.d. | n.d. | n.d. | n.d. | n.d. | n.d. |
| 14 | n.d. | n.d. | n.d. | n.d. | n.d. | n.d. |
| 15 | n.d. | n.d. | n.d. | n.d. | n.d. | n.d. |
| 16 | n.d. | n.d. | n.d. | n.d. | n.d. | n.d. |
| 17 | n.d. | n.d. | n.d. | n.d. | n.d. | n.d. |
| 18 | n.d. | n.d. | n.d. | n.d. | n.d. | n.d. |
| 19 | n.d. | n.d. | n.d. | n.d. | n.d. | n.d. |
| 20 | n.d. | n.d. | n.d. | n.d. | n.d. | n.d. |

TABLE 9
Apparent hydrodynamic radius (Rₕ) and Scattering Intensity
(I) and the Mass Contribution (M) as determined with
DLS for samples prepared in DLS buffer system B.

| | apparent Rₕ and SD | | I and SD | | M and SD | |
|---|---|---|---|---|---|---|
| SEQ | [nm] | | [%] | | [%] | |
| ID NO | 0 weeks | 4 weeks | 0 weeks | 4 weeks | 0 weeks | 4 weeks |
| 9 | 1.85 ± 0.04 | 1.73 ± 0.03 | 82 ± 3 | 97 ± 4 | 100 ± 0 | 100 ± 0 |

### Example 10: In Vitro Data for the Human GLP-1 and GIP Receptors (HEK-293 Cell Lines Over-Expressing Receptors)

Potencies of peptidic compounds at the human GLP-1 or GIP receptors were determined by exposing cells expressing human GIP receptor (hGIPR) or human GLP-1 receptor (hGLP-1R) to the listed compounds at increasing concentrations and measuring the cAMP generated as described in Methods in the presence of 0.1% HSA or without albumin (0% HSA).

The results are shown in Table 10.

TABLE 10
EC50 values at human GLP-1 and GIP receptors (indicated
in pM) - HEK-293 cell lines over-expressing receptors

| | EC50 | EC50 | EC50 |
|---|---|---|---|
| SEQ ID | hGLP-1R | hGIPR | hGIPR |
| NO | 0% HSA [pM] | 0% HSA [pM] | 0.1% HSA [pM] |
| 4 | >10000 | 0.12 | 0.88 |
| 5 | >10000 | 0.16 | 1.75 |
| 6 | >10000 | 0.17 | 1.05 |
| 7 | >10000 | 0.17 | 2.86 |
| 8 | >10000 | 0.19 | 1.37 |
| 9 | >10000 | 0.19 | 1.94 |
| 10 | >10000 | 0.25 | 5.37 |
| 11 | >10000 | 0.19 | 2.24 |
| 12 | >10000 | 0.26 | 2.38 |
| 13 | >10000 | 0.20 | 1.76 |
| 14 | >10000 | 0.23 | 1.56 |
| 15 | >10000 | 0.34 | 7.65 |
| 16 | >10000 | 0.19 | 2.55 |
| 17 | >10000 | 0.29 | 3.39 |
| 18 | n.d. | n.d. | n.d. |
| 19 | n.d. | n.d. | n.d. |
| 20 | n.d. | n.d. | n.d. |
| 1 | >10000 | 0.36 | 0.12 |
| 2 | 0.70 | >10000 | >10000 |

### Example 11: In Vitro Data for the Human GIP Receptor (Human Adipocytes)

Potencies of peptidic compounds at the human GIP receptor were determined by exposing cells expressing human GIP receptor (human adipocytes) to the listed compounds at increasing concentrations and measuring the cAMP generated as described in Methods.

The results are shown in Table 11.

TABLE 11
EC50 and Emax values at human GIP receptors
(indicated in nM and %, respectively)

| SEQ ID | hGIPR | hGIPR |
|---|---|---|
| NO | EC50 [nM] | Emax [%] |
| 4 | 0.5 | 116 |
| 5 | 0.5 | 137 |
| 6 | 0.3 | 112 |
| 7 | 0.3 | 121 |
| 8 | 0.5 | 116 |
| 9 | 1.4 | 125 |
| 10 | 0.4 | 127 |
| 11 | 1.0 | 122 |
| 12 | n.d. | n.d. |
| 13 | n.d. | n.d. |
| 14 | n.d. | n.d. |
| 15 | n.d. | n.d. |
| 16 | n.d. | n.d. |
| 17 | n.d. | n.d. |
| 18 | n.d. | n.d. |
| 19 | n.d. | n.d. |
| 20 | n.d. | n.d. |
| 1 | 4.6 | 100 |
| 2 | not active | — |

### Example 12: In Vitro Affinity Data for the Human GLP-1 and GIP Receptors (Binding Assay)

Affinity of peptidic compounds to the human GIP receptor and the human GLP-1 receptor were determined as described in Methods.

The results are shown in Table 12.

TABLE 12
IC50 values at human GIP and GLP-1 receptors (indicated in nM)

| SEQ ID | IC50 hGIPR | IC50 hGLP-1R |
|---|---|---|
| NO | [nM] | [nM] |
| 4 | 0.2 | 144 |
| 5 | 0.1 | 145 |
| 6 | 0.1 | 173 |
| 7 | 0.2 | 105 |
| 8 | 0.2 | 186 |
| 9 | 0.9 | 307 |
| 10 | 0.3 | 223 |
| 11 | 0.5 | 288 |
| 12 | n.d. | n.d. |
| 13 | n.d. | n.d. |
| 14 | n.d. | n.d. |
| 15 | n.d. | n.d. |
| 16 | n.d. | n.d. |
| 17 | n.d. | n.d. |
| 18 | n.d. | n.d. |
| 19 | n.d. | n.d. |
| 20 | n.d. | n.d. |
| 1 | 3.13 | >3000 |
| 2 | >10000 | 1.44 |

### Example 13: Pharmacokinetic Testing

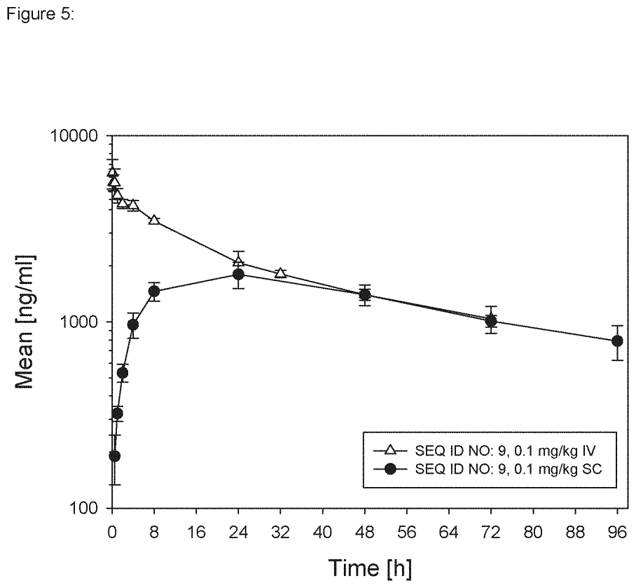

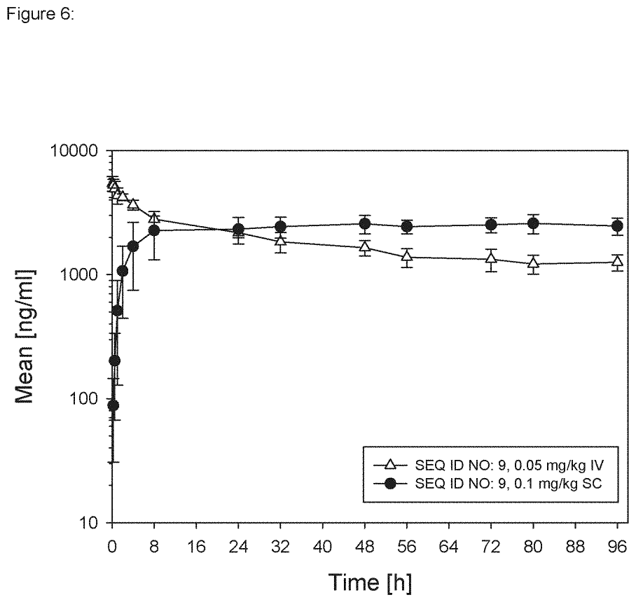

Pharmacokinetic profiles were determined as described in Methods. Calculated T_{1/2} and Cmax values are shown in Table 13 to 16, plasma levels over time are shown in FIGS. 5 and 6.

TABLE 13
Pharmacokinetic profiles in mice.

| SEQ | | T_{1/2} | Cmax | AUClast | CI | F |
|---|---|---|---|---|---|---|
| ID NO | Treatment | [h] | [ng/ml] | [h*ng/ml] | [L/h/kg] | [%] |
| 9 | 0.25 mg/kg | 8.0 | 3920 | 25700 | 0.00959 | |
| | i.v. | | | | | |
| 9 | 0.5 mg/kg | 7.3 | 2190 | 42200 | | 82 |
| | s.c. | | | | | |

TABLE 14
Pharmacokinetic profiles in rats.

| SEQ | | T_{1/2} | Cmax | AUClast | CI | F |
|---|---|---|---|---|---|---|
| ID NO | Treatment | [h] | [ng/ml] | [h*ng/ml] | [L/h/kg] | [%] |
| 4 | 0.25 mg/kg | 12.3 | 6890 | 50200 | 0.00468 | |
| | i.v. | | | | | |
| 4 | 0.5 mg/kg | 16.5 | 1270 | 44400 | — | 52 |
| | s.c. | | | | | |
| 9 | 0.25 mg/kg | 14.8 | 5600 | 54500 | 0.00419 | |
| | i.v. | | | | | |
| 9 | 0.5 mg/kg | 16.9 | 2490 | 72900 | — | 73 |
| | s.c. | | | | | |

TABLE 15
Pharmacokinetic profiles in monkeys.

| SEQ | | T_{1/2} | Cmax | AUClast | CI | F |
|---|---|---|---|---|---|---|
| ID NO | Treatment | [h] | [ng/ml] | [h*ng/ml] | [L/h/kg] | [%] |
| 9 | 0.1 mg/kg | 51.4 | 6740 | 149000 | 0.000445 | |
| | i.v. | | | | | |
| 9 | 0.1 mg/kg | 58.4 | 1800 | 122000 | | 83 |
| | s.c. | | | | | |

TABLE 16
Pharmacokinetic profiles in minipigs.

| SEQ | | T_{1/2} | Cmax | AUClast | CI | F |
|---|---|---|---|---|---|---|
| ID NO | Treatment | [h] | [ng/ml] | [h*ng/ml] | [L/h/kg] | [%] |
| 9 | 0.05 mg/kg | 75.8 | 5530 | 177000 | 0.00016 | |
| | i.v. | | | | | |
| 9 | 0.1 mg/kg | Not | 3080 | 228000 | | 64 |
| | s.c. | calc.* | | | | |

*After s.c. administration, the terminal half-life in plasma could not be determined, since the elimination phase was not reached within the 96-h study timeframe.

### Example 14: Acute Effects of Subcutaneous Treatment of SEQ ID NO: 9 on Blood Glucose Excursion and Glucose Tolerance During an ipGTT in C57Bl/6 Mice

Male C57Bl/6NCrl mice were fed overnight and on the following morning brought to the lab with food removed but ad libitum access to water. Six groups of mice (N=7-8 mice per group) were treated once with a subcutaneous injection of vehicle, increasing doses of the GIPR agonist SEQ ID NO: 9 (3, 10, 30 or 100 nmol/kg) or 10 nmol/kg semaglutide as positive control. The applied volume was 5 ml/kg and the dose was adjusted to the most recent body mass recording of each individual that was taken in the morning. The dosing was initiated and completed between 06:30 and 07:00 AM. Six h after dosing mice were challenged with an intraperitoneal bolus injection of a glucose solution and dose-dependent pharmacodynamic efficacy on blood glucose lowering and glucose tolerance improvement of the GIPR agonist was analyzed compared to the vehicle group as well as the semaglutide positive control.

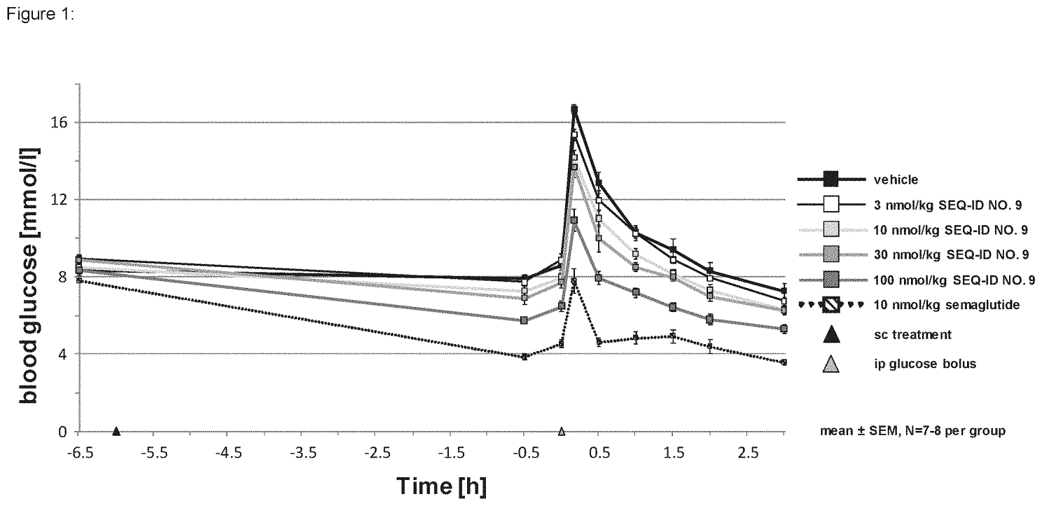

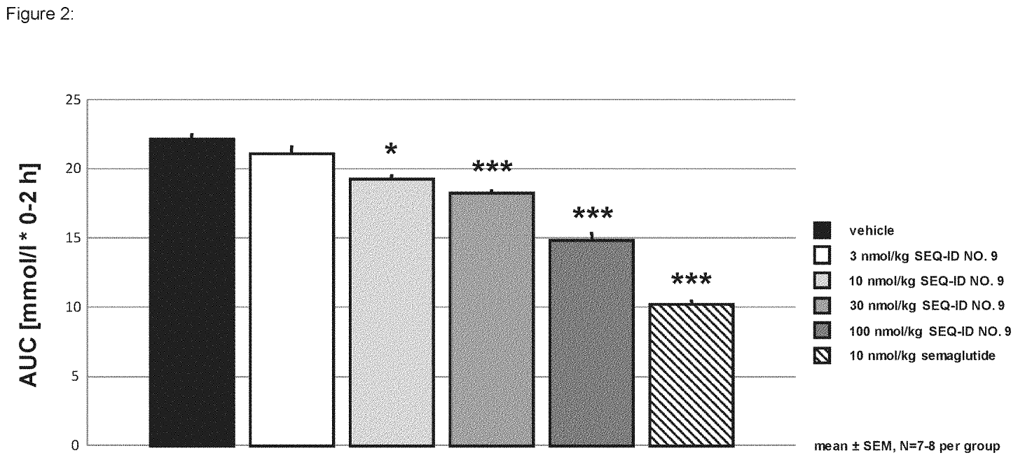

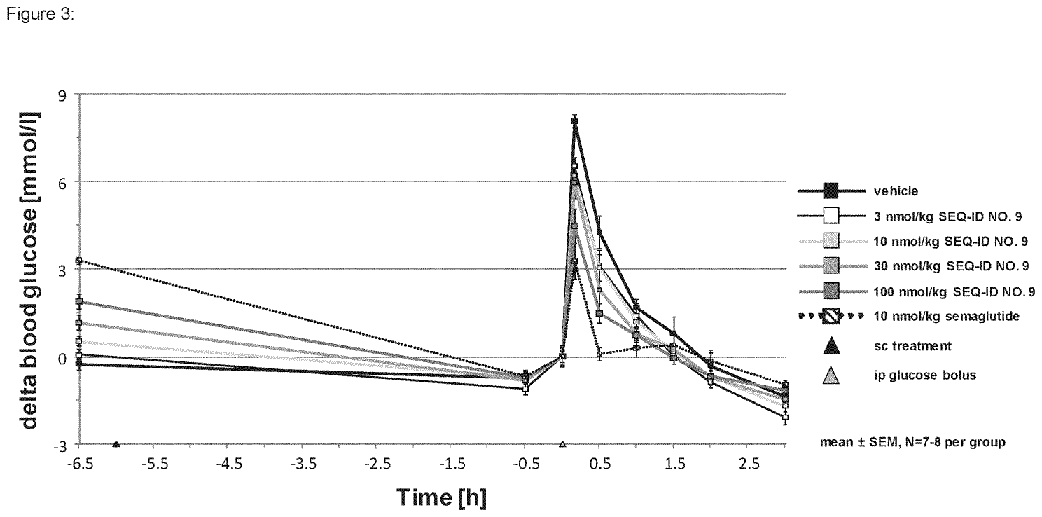

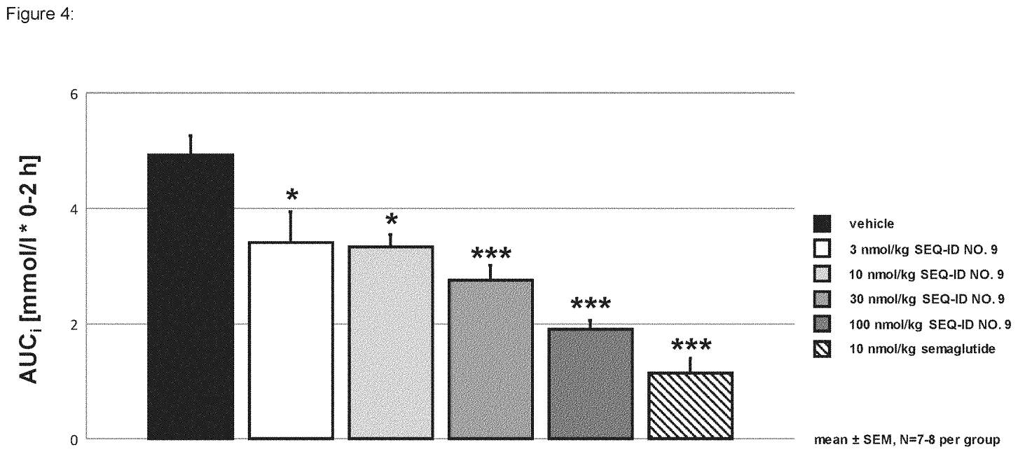

When compared to the vehicle group single dose treatment with the GIPR agonist SEQ ID NO: 9 induced a significant and dose-dependent improvement of i.p. glucose tolerance in C57Bl/6 mice after i.p. glucose load with a minimal effective dose in the range of 3-10 nmol/kg as indicated by the observed reduction of either the AUC analysis data on raw blood glucose concentration (p=0.0045 for 10 nmol/kg dose, see Table 17 and FIGS. 1, 2) or the incremental AUCᵢ analysis on baseline corrected blood glucose concentration values (p=0.0058 for 3 nmol/kg dose, see Table 18, FIGS. 3, 4).

TABLE 17
Acute effects of increasing doses of subcutaneous
treatment of SEQ ID NO: 9 (3, 10, 30 or 100 nmol/kg) on
blood glucose excursion during an ipGTT in C57BI/6 mice
as analysed by an area under curve (AUC) analysis in the
time period from t = 0 h (time point of i.p. glucose challenge)
towards t = 2 h (after i.p. glucose challenge). Data are
means ± SEM. N = 7-8 mice per group. One-way ANOVA,
multiple comparisons versus vehicle (Dunnett's Method).

| Example (Dose) | Blood glucose concentration AUC |
|---|---|
| (0, 3, 10, 30 or 100 nmol/kg) | (mmol/L* 0-2 h) |
| vehicle (N = 8) | +22.15 ± 0.662 |
| SEQ ID NO: 9 (N = 8) | +21.07 ± 0.544 |
| (3 nmol/kg) | p = 0.5479 |
| SEQ ID NO: 9 (N = 8) | +19.28 ± 0.518 |
| (10 nmol/kg) | p = 0.0045 |
| SEQ ID NO: 9 (N = 8) | +18.20 ± 0.662 |
| (30 nmol/kg) | p < 0.0001 |
| SEQ ID NO: 9 (N = 8) | +14.81 ± 0.533 |
| (100 nmol/kg) | p < 0.0001 |
| semaglutide (N = 7) | +10.21 ± 0.503 |
| (10 nmol/kg) | p < 0.0001 |

TABLE 18
Acute effects of increasing doses of subcutaneous treatment of
SEQ ID NO: 9 (3, 10, 30 or 100 nmol/kg) on blood glucose excursion
during an ipGTT in C57BI/6 mice as analysed by an incremental
area under curve (AUCᵢ) analysis on delta blood glucose excursion
data in the time period from t = 0 h (time point of i.p. glucose
challenge) towards t = 2 h (after i.p. glucose challenge). Data are
means ± SEM. N = 7-8 mice per group. One-way ANOVA,
multiple comparisons versus vehicle (Dunnett's Method).

| Example (Dose) | Incremental blood glucose |
|---|---|
| (0, 3, 10, 30 or 100 nmol/kg) | concentration AUCᵢ (mmol/L* 0-2 h) |
| vehicle (N = 8) | +4.93 ± 0.324 |
| SEQ ID NO: 9 (N = 8) | +3.41 ± 0.520 |
| (3 nmol/kg) | p = 0.0058 |
| SEQ ID NO: 9 (N = 8) | +3.33 ± 0.210 |
| (10 nmol/kg) | p = 0.0034 |
| SEQ ID NO: 9 (N = 8) | +2.76 ± 0.268 |
| (30 nmol/kg) | p < 0.0001 |
| SEQ ID NO: 9 (N = 8) | +1.90 ± 0.158 |
| (100 nmol/kg) | p < 0.0001 |
| semaglutide (N = 7) | +1.15 ± 0.255 |
| (10 nmol/kg) | p < 0.0001 |

The invention is further characterized by the following items:

Item 1. Compounds of the formula I

I
R¹HN-Tyr-Aib-Glu-Gly-Thr-Phe-Ile-Ser-Asp-
Leu-Ser-Ile-Aib-X14-Asp-Arg-Ile-His
Gln-X20-Glu-Phe-Ile-Glu-Trp-Leu-Leu-Ala-
Gln-Gly-Pro-Ser-Ser-Gly-Ala-Pro-
Pro-Pro-Ser-R²

wherein
R¹ is H or C₁-C₄-alkyl
X14 represents Lys wherein the —NH₂ side chain group is functionalized by-Z1-Z2-C(O)—R⁵, wherein
   —Z1-Z2- represents a linker in all stereoisomeric forms and
   R⁵ is a moiety comprising up to 70 carbon atoms and heteroatoms selected from N and O,
X20 represents an amino acid residue selected from Glu and Aib,
R² is NH₂ or OH,
or a salt or solvate thereof.

Item 2. Compounds of item 1, which are capable of activating the human GIP receptor.

Item 3. Compounds of items 1 to 2, which are an agonist at the human GIP receptor.

Item 4. Compounds of items 1 to 3, which are capable of activating the human GIP receptor in an assay with whole cells expressing the human GIP receptor.

Item 5. Compounds of items 1 to 4, having an EC50 for hGIP receptor as determined by the method of Example 10 without HSA of 10 pM or less.

Item 6. Compounds of items 1 to 4, having an EC50 for hGIP receptor as determined by the method of Example 10 without HSA of 5 pM or less.

Item 7. Compounds of items 1 to 4, having an EC50 for hGIP receptor as determined by the method of Example 10 without HSA of 1 pM or less.

Item 8. Compounds of items 1 to 4, having an EC50 for hGIP receptor as determined by the method of Example 10 without HSA of 0.36 pM or less.

Item 9. Compounds of items 1 to 8, having a lower EC50 for hGIP receptor than at the human GLP-1 receptor receptor.

Item 10. Compounds of any one of items 1 to 9, having an EC50 for hGLP-1 receptor as determined by the method of Example 10 without HSA of 100 pM or more.

Item 11. Compounds of any one of items 1 to 9, having an EC50 for hGLP-1 receptor as determined by the method of Example 10 without HSA of 1000 pM or more.

Item 12. Compounds of any one of items 1 to 9, having an EC50 for hGLP-1 receptor as determined by the method of Example 10 without HSA of 5000 pM or more.

Item 13. Compounds of any one of items 1 to 9, having an EC50 for hGLP-1 receptor as determined by the method of Example 10 without HSA of 10000 pM or more.

Item 14. Compounds of any one of items 1 to 13 having an EC50 for hGIP receptor as determined by the method of Example 11 of 10 nM or less.

Item 15. Compounds of any one of items 1 to 13 having an EC50 for hGIP receptor as determined by the method of Example 11 of 8 nM or less.

Item 16. Compounds of any one of items 1 to 13 having an EC50 for hGIP receptor as determined by the method of Example 11 of 4.6 nM or less.

Item 17. Compounds of any one of items 1 to 13 having an EC50 for hGIP receptor as determined by the method of Example 11 of 2 nM or less.

Item 18. Compounds of any one of items 1 to 17 binding to the hGIP receptor as determined using the method of Example 12 with an IC50 of 10 nM or less.

Item 19. Compounds of any one of items 1 to 17 binding to the hGIP receptor as determined using the method of Example 12 with an IC50 of 8 nM or less.

Item 20. Compounds of any one of items 1 to 17 binding to the hGIP receptor as determined using the method of Example 12 with an IC50 of 5 nM or less.

Item 21. Compounds of any one of items 1 to 17 binding to the hGIP receptor as determined using the method of Example 12 with an IC50 of 3.13 nM or less.

Item 22. Compounds of any one of items 1 to 17 binding to the hGIP receptor as determined using the method of Example 12 with an IC50 of 1 nM or less.

Item 23. Compounds of any one of items 1 to 22 binding to the hGLP-1 receptor as determined using the method of Example 12 with an IC50 of 10 nM or more.

Item 24. Compounds of any one of items 1 to 22 binding to the hGLP-1 receptor as determined using the method of Example 12 with an IC50 of 50 nM or more.

Item 25. Compounds of any one of items 1 to 22 binding to the hGLP-1 receptor as determined using the method of Example 12 with an IC50 of 100 nM or more.

Item 26. Compounds of any one of items 1 to 25 having a high solubility at physiological pH values.

Item 27. Compounds of any one of items 1 to 25 having a high solubility at pH 6 to 8.

Item 28. Compounds of any one of items 1 to 25 having a high solubility at pH 6 to 8 at 25° C. or 40° C.

Item 29. Compounds of any one of items 1 to 25 having a high solubility at pH 7.4 at 25° C. or 40° C.

Item 30. Compounds of any one of items 1 to 25 having a high solubility of at least 1 mg/ml.

Item 31. Compounds of any one of items 1 to 25 having a high solubility of at least 5 mg/ml.

Item 32. Compounds of any one of items 1 to 25 having a high solubility of at least 10 mg/ml.

Item 33. Compounds of any one of items 1 to 25 having a high solubility of at least 30 mg/ml.

Item 34. Compounds of any one of items 1 to 33 having a high solubility at physiological pH values in the presence of an antimicrobial preservative like phenol or m-cresol.

Item 35. Compounds of any one of items 1 to 33 having a high solubility at physiological pH values in the presence of an antimicrobial preservative like phenol or m-cresol, at an acidity range from pH 7 to 8.

Item 36. Compounds of any one of items 1 to 33 having a high solubility at physiological pH values in the presence of an antimicrobial preservative like phenol or m-cresol at pH 7.4 at 25° C. or 40° C.

Item 37. Compounds of any one of items 1 to 33 having a high solubility at physiological pH values in the presence of an antimicrobial preservative like phenol or m-cresol of at least 1 mg/ml.

Item 38. Compounds of any one of items 1 to 33 having a high solubility at physiological pH values in the presence of an antimicrobial preservative like phenol or m-cresol of at least 5 mg/ml.

Item 39. Compounds of any one of items 1 to 33 having a high solubility at physiological pH values in the presence of an antimicrobial preservative like phenol or m-cresol of at least 10 mg/ml.

Item 40. Compounds of any one of items 1 to 39 having a high chemical stability when stored in solution.

Item 41. Compounds of any one of items 1 to 39 having a high chemical stability after 28 days at 40° C. in solution at pH 7.4 the relative purity loss is no more than 20%.

Item 42. Compounds of any one of items 1 to 39 having a high chemical stability after 28 days at 40° C. in solution at pH 7.4 the relative purity loss is no more than 15%.

Item 43. Compounds of any one of items 1 to 39 having a high chemical stability after 28 days at 40° C. in solution at pH 7.4 the relative purity loss is no more than 12%.

Item 44. Compounds of any one of items 1 to 43 having a high chemical stability when stored in solution in the presence of an antimicrobial preservative like phenol or m-cresol.

Item 45. Compounds of any one of items 1 to 43 having a high chemical stability in the presence of an antimicrobial preservative like phenol or m-cresol, after 28 days at 40° C. in solution at pH 7.4 the relative purity loss is no more than 20%,

Item 46. Compounds of any one of items 1 to 43 having a high chemical stability in the presence of an antimicrobial preservative like phenol or m-cresol, after 28 days at 40° C. in solution at pH 7.4 the relative purity loss is no more than 15%,

Item 47. Compounds of any one of items 1 to 43 having a high chemical stability in the presence of an antimicrobial preservative like phenol or m-cresol, after 28 days at 40° C. in solution at pH 7.4 the relative purity loss is no more than 12%.

Item 48. Compounds of any one of items 1 to 47 having a high physical stability.

Item 49. Compounds of any one of items 1 to 48 which do not show an increase in fluorescence intensity with Thioflavin T as fluorescence probe at concentrations of 3 mg/ml.

Item 50. Compounds of any one of items 1 to 48 which do not show an increase in fluorescence intensity with Thioflavin T as fluorescence probe at concentrations of 3 mg/ml at a pH range from pH 6 to 8.

Item 51. Compounds of any one of items 1 to 48 which do not show an increase in fluorescence intensity with Thioflavin T as fluorescence probe at concentrations of 3 mg/ml at pH 7.4 at 37° C. over 5 h.

Item 52. Compounds of any one of items 1 to 48 which do not show an increase in fluorescence intensity with Thioflavin T as fluorescence probe at concentrations of 3 mg/ml at pH 7.4 at 37° C. over 10 h.

Item 53. Compounds of any one of items 1 to 48 which do not show an increase in fluorescence intensity with Thioflavin T as fluorescence probe at concentrations of 3 mg/ml at pH 7.4 at 37° C. over 30 h.

Item 54. Compounds of any one of items 1 to 48 which do not show an increase in fluorescence intensity with Thioflavin T as fluorescence probe at concentrations of 3 mg/ml at pH 7.4 at 37° C. over 45 h.

Item 55. Compounds of any one of items 1 to 54 which do not show an increase in fluorescence intensity with Thioflavin T as fluorescence probe at concentrations of 3 mg/ml in the presence of an antimicrobial preservative like phenol or m-cresol.

Item 56. Compounds of any one of items 1 to 54 which do not show an increase in fluorescence intensity with Thioflavin T as fluorescence probe at concentrations of 3 mg/ml in the presence of an antimicrobial preservative like phenol or m-cresol at an acidity range from pH 6 to 8.

Item 57. Compounds of any one of items 1 to 54 which do not show an increase in fluorescence intensity with Thioflavin T as fluorescence probe at concentrations of 3 mg/ml in the presence of an antimicrobial preservative like phenol or m-cresol at pH 7.4 at 37° C. over 5 h.

Item 58. Compounds of any one of items 1 to 54 which do not show an increase in fluorescence intensity with Thioflavin T as fluorescence probe at concentrations of 3 mg/ml in the presence of an antimicrobial preservative like phenol or m-cresol at pH 7.4 at 37° C. over 10 h.

Item 59. Compounds of any one of items 1 to 54 which do not show an increase in fluorescence intensity with Thioflavin T as fluorescence probe at concentrations of 3 mg/ml in the presence of an antimicrobial preservative like phenol or m-cresol at pH 7.4 at 37° C. over 30 h.

Item 60. Compounds of any one of items 1 to 54 which do not show an increase in fluorescence intensity with Thioflavin T as fluorescence probe at concentrations of 3 mg/ml in the presence of an antimicrobial preservative like phenol or m-cresol at pH 7.4 at 37° C. over 45 h.

Item 61. Compounds of any one of items 1 to 60 which have improved pharmacokinetic properties.

Item 62. Compounds of any one of items 1 to 60 which have an increased half-life in vivo.

Item 63. Compounds of any one of items 1 to 60 which have an increased half-life when determined in minipigs.

Item 64. Compounds of any one of items 1 to 60 which have an increased half-life when determined in cynomolgous monkeys.

Item 65. Compounds of any one of items 1 to 64 which have the effect of improving glucose tolerance in vivo as determined in an acute study in mice, such as in Example 14 described herein.

Item 66. Compounds of any one of items 1 to 65 wherein R¹ is H or methyl.

Item 67. Compounds of any one of items 1 to 65 wherein R¹ is H.

Item 68. Compounds of any one of items 1 to 65 wherein R¹ is methyl.

Item 69. Compounds of formula I of any one of items 1 to 65 wherein R² is NH₂.

Item 70. Compounds of formula I of any one of items 1 to 65 wherein R² is OH.

Item 71. Compounds of formula I of any one of items 1 to 70 wherein
X14 represents Lys wherein the —NH₂ side chain group is functionalized with a group-Z1-Z2-C(O)R⁵, wherein
Z1 represents a group selected from AEEA, {AEEA}2, {AEEA}3, Gly, Gly-Gly, {Gly}3, N-MeGly, {N-MeGly}2, {N-MeGly}3;
Z2 represents a group selected from gGlu, or gGlu-gGlu; and
R⁵ represents a group —(CH2)x-COOH, wherein x is an integer from 15 to 22.

Item 72. Compounds of formula I of any one of items 1 to 71 in form of enantiomers of —Z—C(O)—R⁵ groups, either S- or R-enantiomers.

Item 73. Compounds of formula I of any one of items 1 to 71 wherein
X14 represents Lys wherein the —NH₂ side chain group is functionalized with a group-Z1-Z2-C(O)R⁵, wherein
Z1 represents a group selected from {AEEA}2, {AEEA}3, {Gly}3, {N-MeGly}3; Z2 represents a group selected from gGlu, or gGlu-gGlu; and
R⁵ represents a group —(CH2)x-COOH, wherein x is an integer from 15 to 22.

Item 74. Compounds of formula I of any one of items 1 to 71 wherein
X14 represents Lys wherein the —NH₂ side chain group is functionalized with a group-Z1-Z2-C(O)R⁵, wherein
Z1 represents a group selected from AEEA, {AEEA}2, {AEEA}3, Gly, Gly-Gly, {Gly}3, N-MeGly, {N-MeGly}2, {N-MeGly}3;
Z2 represents a group selected from gGlu, or gGlu-gGlu; and
R⁵ represents 17-carboxy-1-oxoheptadecyl or 19-carboxy-1-oxononadecyl.

Item 75. Compounds of formula I of any one of items 1 to 71 wherein
X14 represents Lys wherein the —NH₂ side chain group is functionalized with a group-Z1-Z2-C(O)R⁵, wherein
Z1 represents a group selected from {AEEA}2, {AEEA}3, {Gly}3, {N-MeGly}3;
Z2 represents a group selected from gGlu, or gGlu-gGlu; and
R⁵ represents 17-carboxy-1-oxoheptadecyl or 19-carboxy-1-oxononadecyl.

Item 76. Compounds of formula I of any one of items 1 to 71 wherein
[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl-,
[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl-,
[2-[2-[2-[[2-[2-[2-[[2-[2-[2-[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl-,
[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]-ethoxy]acetyl]amino]ethoxy]ethoxy]acetyl-,
[2-[2-[2-[[2-[2-[2-[[(4S)-4-carboxy-4-[[(4S)-4-carboxy-4-(19-carboxynonadecanoylamino)butanoyl]amino]butanoyl]amino]ethoxy]-ethoxy]acetyl]amino]ethoxy]ethoxy]acetyl-,
[2-[[2-[[2-[[(4S)-4-carboxy-4-(17-carboxyheptadecanoyl-amino)butanoyl]amino]acetyl]amino]acetyl]amino]acetyl]-,
[2-[methyl-[2-[methyl-[2-[methyl-[(4S)-4-carboxy-4-(17-carboxy-heptadecanoylamino)butanoyl]amino]acetyl]amino]acetyl]amino]acetyl].

Item 77. Compounds of formula I of any one of items 1 to 71 wherein
X14 represents Lys wherein the —NH₂ side chain group is functionalized with a group-Z1-Z2-C(O)R⁵, wherein
Z1-Z2- represents AEEA-AEEA-gGlu and
R⁵ represents a group selected from pentadecenoyl, heptadecenoyl, nonadecanoyl, 17-carboxy-1-oxoheptadecyl, or 19-carboxy-1-oxononadecyl.

Item 78. Compounds of formula I of any one of items 1 to 71 wherein
X14 represents Lys wherein the —NH₂ side chain group is functionalized with a group-Z1-Z2-C(O)R⁵, wherein
—Z1-Z2- represents AEEA-AEEA-gGlu- and
R⁵ represents a group selected from 17-carboxy-1-oxoheptadecyl, or 19-carboxy-1-oxononadecyl.

Item 79. Compounds of formula I of any one of items 1 to 71 wherein
X14 represents Lys wherein the —NH₂ side chain group is functionalized by [2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl- or [2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl-.

Item 80. Compounds of formula I of any one of items 1 to 71 wherein
X14 represents Lys wherein the —NH₂ side chain group is functionalized by 2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl-.

Item 81. Compounds of formula I of any one of items 1 to 71 wherein
X14 represents Lys wherein the —NH₂ side chain group is functionalized with a group-Z1-Z2-C(O)R⁵, wherein
—Z1-Z2- represents AEEA-AEEA-AEEA-gGlu- and
R⁵ represents a group selected from 17-carboxy-1-oxoheptadecyl, or 19-carboxy-1-oxononadecyl.

Item 82. Compounds of formula I of any one of items 1 to 71 wherein
X14 represents Lys wherein the —NH₂ side chain group is functionalized with a group-Z1-Z2-C(O)R⁵, wherein
—Z1-Z2- represents AEEA-AEEA-gGlu-gGlu- and
R⁵ represents a group selected from 17-carboxy-1-oxoheptadecyl, or 19-carboxy-1-oxononadecyl.

Item 83. Compounds of formula I of any one of items 1 to 71 wherein
X14 represents Lys wherein the —NH₂ side chain group is functionalized by a group selected from
[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl-,
[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl-,
[2-[2-[2-[[2-[2-[2-[[2-[2-[2-[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl-,
[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]-ethoxy]acetyl]amino]ethoxy]ethoxy]acetyl-,
[2-[2-[2-[[2-[2-[2-[[(4S)-4-carboxy-4-[[(4S)-4-carboxy-4-(19-carboxynonadecanoylamino)butanoyl]amino]butanoyl]amino]ethoxy]-ethoxy]acetyl]amino]ethoxy]ethoxy]acetyl-,
[2-[[2-[[2-[[(4S)-4-carboxy-4-(17-carboxyheptadecanoyl-amino)butanoyl]amino]acetyl]amino]acetyl]amino]acetyl]-,
[2-[methyl-[2-[methyl-[2-[methyl-[(4S)-4-carboxy-4-(17-carboxy-heptadecanoylamino)butanoyl]amino]acetyl]amino]acetyl]amino]acetyl],
   R² represents NH₂,
   or a salt or solvate thereof.

Item 84. Compounds of formula I of any one of items 1 to 71 wherein
X14 represents Lys wherein the —NH₂ side chain group is functionalized by a group selected from
[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl-,
[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl-,
[2-[2-[2-[[2-[2-[2-[[2-[2-[2-[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl-,
[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]-ethoxy]acetyl]amino]ethoxy]ethoxy]acetyl-,
[2-[2-[2-[[2-[2-[2-[[(4S)-4-carboxy-4-[[(4S)-4-carboxy-4-(19-carboxynonadecanoylamino)butanoyl]amino]butanoyl]amino]ethoxy]-ethoxy]acetyl]amino]ethoxy]ethoxy]acetyl-,
[2-[[2-[[2-[[(4S)-4-carboxy-4-(17-carboxyheptadecanoyl-amino)butanoyl]amino]acetyl]amino]acetyl]amino]acetyl]-,
[2-[methyl-[2-[methyl-[2-[methyl-[(4S)-4-carboxy-4-(17-carboxy-heptadecanoylamino)butanoyl]amino]acetyl]amino]acetyl]amino]acetyl],
   R² represents OH,
   or a salt or solvate thereof.

Item 85. Compounds of formula I of any one of items 1 to 65 and 71 to 84 wherein
R¹ is selected from H or methyl,
X20 is Glu,
R² is NH₂ or OH,
or a salt or solvate thereof.

Item 86. Compounds of formula I of any one of items 1 to 65 and 71 to 84 wherein
R¹ is H,
X20 is Glu,
R² is NH₂ or OH,
or a salt or solvate thereof.

Item 87. Compounds of formula I of any one of items 1 to 65 and 71 to 84 wherein
R¹ is H,
X20 is Glu,
R² is NH₂,
or a salt or solvate thereof.

Item 88. Compounds of formula I of any one of items 1 to 65 and 71 to 84 wherein
R¹ is H,
X20 is Glu,
R² is OH,
or a salt or solvate thereof.

Item 89. Compounds of formula I of any one of items 1 to 65 and 71 to 84 wherein
R¹ is methyl,
X20 is Glu,
R² is NH₂ or OH,
or a salt or solvate thereof.

Item 90. Compounds of formula I of any one of items 1 to 65 and 71 to 84 wherein
R¹ is methyl,
X20 is Glu,
R² is NH₂,
or a salt or solvate thereof.

Item 91. Compounds of formula I of any one of items 1 to 65 and 71 to 84 wherein
R¹ is methyl,
X20 is Glu,
R² is OH,
or a salt or solvate thereof.

Item 92. Compounds of formula I of any one of items 1 to 65 and 71 to 84 wherein
R¹ is selected from H or methyl,
X20 is Aib,
R² is NH₂ or OH,
or a salt or solvate thereof.

Item 93. Compounds of formula I of any one of items 1 to 65 and 71 to 84 wherein
R¹ is H,
X20 is Aib,
R² is NH₂ or OH,
or a salt or solvate thereof.

Item 94. Compounds of formula I of any one of items 1 to 65 and 71 to 84 wherein
R¹ is H,
X20 is Aib,
R² is NH₂,
or a salt or solvate thereof.

Item 95. Compounds of formula I of any one of items 1 to 65 and 71 to 84 wherein
R¹ is H,
X20 is Aib,
R² is OH,
or a salt or solvate thereof.

Item 96. Compounds of formula I of any one of items 1 to 65 and 71 to 84 wherein
R¹ is methyl,
X20 is Aib,
R² is NH₂ or OH,
or a salt or solvate thereof.

Item 97. Compounds of formula I of any one of items 1 to 65 and 71 to 84 wherein
R¹ is methyl,
X20 is Aib,
R² is NH₂,
or a salt or solvate thereof.

Item 98. Compounds of formula I of any one of items 1 to 65 and 71 to 84 wherein
R¹ is methyl,
X20 is Aib,
R² is OH,
or a salt or solvate thereof.

Item 99. Compounds of SEQ ID NO: 9-17 as well as salts or solvates thereof.

Item 100. Compounds of SEQ ID NO: 4-8 and 18-20 as well as salts or solvates thereof.

Item 101. Compounds of SEQ ID NO: 11 and 20 as well as salts or solvates thereof.

Item 102. Compounds of SEQ ID NO: 8 and 13 as well as salts or solvates thereof.

Item 103. Compounds of SEQ ID NO: 4-10 and 12-19 as well as salts or solvates thereof.

Item 104. Compound of SEQ ID NO: 4-17 as well as salts or solvates thereof.

Item 105. Compound of SEQ ID NO: 4-20 as well as salts or solvates thereof.

Item 106. Compound of SEQ ID NO: 4 as well as salts or solvates thereof.

Item 107. Compound of SEQ ID NO: 7 as well as salts or solvates thereof.

Item 108. Compound of SEQ ID NO: 9 as well as salts or solvates thereof.

Item 109. Composition comprising a compound of formula I of any one of items 1 to 108 in admixture with a carrier.

Item 110. Composition comprising a compound of formula I of any one of items 1 to 108 in admixture with a carrier.

Item 111. Composition comprising a compound of formula I of any one of items 1 to 108 in admixture with a carrier wherein the composition is a pharmaceutically acceptable composition and the carrier is a pharmaceutically acceptable carrier.

Item 112. Composition of any one of items 1 to 111 for use in a method of medical treatment.

Item 113. Composition of any one of items 1 to 112 for use in a method of medical treatment in human medicine.

Item 114. Compounds of formula I of any one of items 1 to 113 for the treatment or prevention of diseases or disorders caused by, associated with and/or accompanied by disturbances in carbohydrate and/or lipid metabolism.

Item 115. Compounds of formula I of any one of items 1 to 113 for the treatment or prevention of hyperglycemia, type 2 diabetes, impaired glucose tolerance, type 1 diabetes, obesity and metabolic syndrome.

Item 116. Compounds of formula I of any one of items 1 to 113 for the treatment or prevention of degenerative diseases.

Item 117. Compounds of formula I of any one of items 1 to 113 for the treatment or prevention of neurodegenerative diseases.

Item 118. Compounds of formula I of any one of items 1 to 113 for the prevention of weight gain or promoting weight loss.

Item 119. Compounds of formula I of any one of items 1 to 113 for the decrease in food intake, resulting in the observed effect on body weight.

Item 120. Compounds of formula I of any one of items 1 to 113 for the treatment or prevention of obesity, morbid obesity, obesity linked inflammation, obesity linked gallbladder disease or obesity induced sleep apnoea.

Item 121. Compounds of formula I of any one of items 1 to 113 for the treatment or prevention of metabolic syndrome, diabetes, hypertension, atherogenic dyslipidemia, atherosclerosis, arteriosclerosis, coronary heart disease, or stroke.

Item 122. Compounds of formula I of any one of items 1 to 113 for delaying or preventing disease progression in type 2 diabetes, treating metabolic syndrome, treating obesity or preventing overweight, for decreasing food intake, reducing body weight, delaying the progression from impaired glucose tolerance (IGT) to type 2 diabetes; delaying the progression from type 2 diabetes to insulin-requiring diabetes and hepatic steatosis.

Item 123. Compounds of formula I of any one of items 1 to 113 for treatment of glucose intolerance, insulin resistance, pre-diabetes, increased fasting glucose (hyperglycemia), type 2 diabetes, hypertension, dyslipidemia, arteriosclerosis, coronary heart disease, peripheral artery disease, stroke or any combination of these individual disease components.

Item 124. Compounds of formula I of any one of items 1 to 113 for control of appetite, feeding and caloric intake, prevention of weight gain, promotion of weight loss, reduction of excess body weight and altogether treatment of obesity, including morbid obesity.

Item 125. Compounds of formula I of any one of items 1 to 113 for simultaneous treatment of diabetes and obesity.

Item 126. Compounds of formula I of any one of items 1 to 113 for the treatment of obesity-linked inflammation, obesity-linked gallbladder disease and obesity-induced sleep apnoea.

Item 127. Compounds of formula I of any one of items 1 to 113 for the treatment of Alzheimer's disease or Parkinson's disease.

Item 128. Compounds of formula I of any one of items 1 to 113 for the treatment or prevention of hyperglycemia, type 2 diabetes, and/or obesity.

Item 129. Compounds of formula I of any one of items 1 to 113 for the reduce of blood glucose level, and/or for the reduce of HbA1c levels of a patient.

Item 130. Compounds of formula I of any one of items 1 to 113 for the reduce of body weight of a patient.

Item 131. Compounds of formula I of any one of items 1 to 113 for the treatment or prevention of osteoporosis.

Item 132. Compounds of formula I of any one of items 1 to 113 for the treatment or prevention preferably non-alcoholic liver-disease (NAFLD) and non-alcoholic steatohepatitis (NASH).

Item 133. Compounds of formula I of any one of items 1 to 113 for the treatment or prevention of nausea and/or vomiting.

Item 134. Kit that comprises a compound of formula (I) of any one of items 1 to 133 in any of its stereoisomeric forms, or a physiologically acceptable salt or solvate thereof, and a set of instructions relating to the use of the compound for the methods described herein.

Item 135. Kit of item 134 comprising one or more inert carriers and/or diluents.

Item 136. Kit of item 134 comprising one or more inert carriers and/or diluents. and comprising one or more other pharmacologically active compounds.

Item 137. Kit of any one of items 134 to 136 comprising a device for application.

Item 138. Kit of any one of items 134 to 137 comprising a syringe, an injection pen or an autoinjector.

Item 139. Kit of any one of items 134 to 138 wherein the device may be provided separate from a pharmaceutical composition or prefilled with the pharmaceutical composition.

Item 140. Combination of a compound of formula I of any one of items 1 to 133 with a antidiabetic agent.

Item 141. Combination of a compound of formula I of any one of items 1 to 133 with Insulin and insulin derivatives.

Item 142. Combination of a compound of formula I of any one of items 1 to 133 with insulin glargine (e.g. Lantus®), higher than 100 U/ml concentrated insulin glargine, e.g. 270-330 U/ml of insulin glargine or 300 U/ml of insulin glargine (e.g. Toujeo®), insulin glulisine (e.g. Apidra®), insulin detemir (e.g. Levemir®), insulin lispro (e.g. Humalog®, Liprolog®), insulin degludec (e.g. DegludecPlus®, IdegLira (NN9068)), insulin aspart and aspart formulations (e.g. NovoLog®), basal insulin and analogues (e.g. LY2605541, LY2963016, NN1436), PEGylated insulin lispro (e.g. LY-275585), long-acting insulins (e.g. NN1436, Insumera (PE0139), AB-101, AB-102, Sensulin LLC), intermediate-acting insulins (e.g. Humulin®N, Novolin®N), fast-acting and short-acting insulins (e.g. Humulin®R, Novolin®R, Linjeta® (VIAject®), PH20 insulin, NN1218, HinsBet®, premixed insulins, SuliXen®, NN1045, insulin plus Symlin®, PE-0139, ACP-002 hydrogel insulin, and oral, inhalable, transdermal and buccal or sublingual insulins (e.g. Exubera®, Nasulin®, Afrezza®, insulin tregopil, TPM-02 insulin, Capsulin®, Oral-lyn®, Cobalamin® oral insulin, ORMD-0801, Oshadi oral insulin, NN1953, NN1954, NN1956, VIAtab®).

Item 143. Combination of a compound of formula I of any one of items 1 to 133 with insulin derivatives which are bonded to albumin or another protein by a bifunctional linker.

Item 144. Combination of a compound of formula I of any one of items 1 to 133 with GLP-1, GLP-1 analogues and GLP-1 receptor agonists, for example: lixisenatide (e.g. Lyxumia®), exenatide (e.g. exendin-4, rExendin-4, Byetta®, Bydureon®, exenatide NexP), liraglutide (e.g. Victoza®), semaglutide (e.g. Ozempic®), taspoglutide, albiglutide, dulaglutide (e.g. Trulicity®), ACP-003, CJC-1134-PC, GSK-2374697, PB-1023, TTP-054, efpeglenatide (HM-11260C), CM-3, GLP-1 Eligen, AB-201, ORMD-0901, NN9924, NN9926, NN9927, Nodexen, Viador-GLP-1, CVX-096, ZYOG-1, ZYD-1, ZP-3022, CAM-2036, DA-3091, DA-15864, ARI-2651, ARI-2255, exenatide-XTEN (VRS-859), exenatide-XTEN+Glucagon-XTEN (VRS-859+AMX-808) and polymer-bound GLP-1 and GLP-1 analogues.

Item 145. Combination of a compound of formula I of any one of items 1 to 133 with dual GLP-1/glucagon receptor agonists, e.g. BHM-034, OAP-189 (PF-05212389, TKS-1225), pegapamodutide (TT-401/402), ZP2929, JNJ64565111 (HM 12525A, LAPS-HMOXM25), MOD-6030, NN9277, LY-3305677, MEDI-0382, MK8521, B1456906, VPD-107, H&D-001A, PB-718, SAR425899 or compounds disclosed in WO2014/056872.

Item 146. Combination of a compound of formula I of any one of items 1 to 133 with dual GLP-1/GIP agonists, e.g. RG-7685 (MAR-701), RG-7697 (MAR-709, NN9709), BHM081, BHM089, BHM098, Tirzepatide (LY3298176), LBT-6030, ZP-1-70, TAK-094, SAR438335 or compounds disclosed in WO2014/096145, WO2014/096148, WO2014/096149, WO2014/096150 and WO2020/023386.

Item 147. Combination of a compound of formula I of any one of items 1 to 133 with triple GLP-1/glucagon/GIP receptor agonists (e.g. Tri-agonist 1706 (NN9423), HM15211).

Item 148. Combination of a compound of formula I of any one of items 1 to 133 with dual GLP-1R agonist/Proprotein convertase subtilisin/kexin type 9 (e.g. MEDI-4166).

Item 149. Combination of a compound of formula I of any one of items 1 to 133 with dual GLP-1/GLP-2 receptor agonists (e.g. ZP-GG-72).

Item 150. Combination of a compound of formula I of any one of items 1 to 133 with dual GLP-1/gastrin agonists (e.g. ZP-3022).

Item 151. Combination of a compound of formula I of any one of items 1 to 133 with peptide YY 3-36 (PYY3-36) or analogues thereof and pancreatic polypeptide (PP) or analogues thereof (e.g. PYY 1562 (NN9747/NN9748)).

Item 152. Combination of a compound of formula I of any one of items 1 to 133 with Calcitonin and calcitonin analogs, amylin and amylin analogues (e.g. pramlintide, Symlin®), dual calcitonin and amylin receptor agonists such as Salmon Calcitonin (e.g. Miacalcic®), davalintide (AC2307), mimylin, AM833 (NN9838), KBP-042, KBP-088, and KBP-089, ZP-4982/ZP-5461, elcatonin.

Item 153. Combination of a compound of formula I of any one of items 1 to 133 with Glucagon-like-peptide 2 (GLP-2), GLP-2 analogues, and GLP-2 receptor agonists, for example: teduglutide (e.g. Gattex®), elsiglutide, glepaglutide, FE-203799, HM15910.

Item 154. Combination of a compound of formula I of any one of items 1 to 133 with Glucagon receptor agonists (e.g. G530S (NN9030), dasiglucagon, HM15136, SAR438544, DIO-901, AMX-808) or antagonists, glucose-dependent insulinotropic polypeptide (GIP) receptor agonists (e.g. ZP-1-98, AC163794) or antagonists (e.g. GIP(3-30)NH₂), ghrelin antagonists or inverse agonists, xenin and analogues thereof.

Item 155. Combination of a compound of formula I of any one of items 1 to 133 with Human fibroblast growth factor 21 (FGF21) and derivatives or analogues such as LY2405319 and NN9499 or other variants of FGF21.

Item 156. Combination of a compound of formula I of any one of items 1 to 133 with Dipeptidyl peptidase-IV (DPP-4) inhibitors.

Item 157. Combination of a compound of formula I of any one of items 1 to 133 with alogliptin (e.g. Nesina®, Kazano®), linagliptin (e.g. Ondero®, Trajenta®, Tradjenta®, Trayenta®), saxagliptin (e.g. Onglyza®, Komboglyze XR®), sitagliptin (e.g. Januvia®, Xelevia®, Tesavel®, Janumet®, Velmetia®, Juvisync®, Janumet XR®), anagliptin, teneligliptin (e.g. Tenelia®), trelagliptin, vildagliptin (e.g. Galvus®, Galvumet®), gemigliptin, omarigliptin, evogliptin, dutogliptin, DA-1229, MK-3102, KM-223, KRP-104, PBL-1427, Pinoxacin hydrochloride, and Ari-2243.

Item 158. Combination of a compound of formula I of any one of items 1 to 133 with Sodium-dependent glucose transporter 2 (SGLT-2) inhibitors.

Item 159. Combination of a compound of formula I of any one of items 1 to 133 with Canagliflozin (e.g. Invokana®), Dapagliflozin (e.g. Forxiga®), Remogliflozin, Sergliflozin, Empagliflozin (e.g. Jardiance®), Ipragliflozin, Tofogliflozin, Luseogliflozin, Ertuglifozin/PF-04971729, RO-4998452, Bexagliflozin (EGT-0001442), SBM-TFC-039, Henagliflozin (SHR3824), Janagliflozin, Tianagliflozin, AST1935, JRP493, HEC-44616.

Item 160. Combination of a compound of formula I of any one of items 1 to 133 with Dual inhibitors of SGLT-1 and SGLT-2 (e.g. sotagliflozin, LX-4211, LIK066), SGLT-1 inhibitors (e.g. LX-2761, Mizagliflozin (KGA-3235)) or SGLT-1 inhibitors in combination with anti-obesity drugs such as ileal bile acid transfer (IBAT) inhibitors (e.g. GSK-1614235 and GSK-2330672).

Item 161. Combination of a compound of formula I of any one of items 1 to 133 with Biguanides.

Item 162. Combination of a compound of formula I of any one of items 1 to 133 with Metformin, Buformin or Phenformin).

Item 163. Combination of a compound of formula I of any one of items 1 to 133 with Thiazolidinediones.

Item 164. Combination of a compound of formula I of any one of items 1 to 133 with Pioglitazone, Rivoglitazone, Rosiglitazone, Troglitazone and lobeglitazone.

Item 165. Combination of a compound of formula I of any one of items 1 to 133 with Peroxisome proliferator-activated receptors (PPAR-)(alpha, gamma or alpha/gamma) agonists or modulators (e.g. saroglitazar (e.g. Lipaglyn®), GFT-505), or PPAR gamma partial agonists (e.g. Int-131).

Item 166. Combination of a compound of formula I of any one of items 1 to 133 with Sulfonylureas (e.g. Tolbutamide, Glibenclamide, Glimepiride (e.g. Amaryl®), Glipizide), Meglitinides (e.g. Nateglinide, Repaglinide, Mitiglinide)

Item 167. Combination of a compound of formula I of any one of items 1 to 133 with Alpha-glucosidase inhibitors (e.g. Acarbose, Miglitol, Voglibose).

Item 168. Combination of a compound of formula I of any one of items 1 to 133 with GPR119 agonists (e.g. GSK-1292263, PSN-821, MBX-2982, APD-597, ARRY-981, ZYG-19, DS-8500, HM-47000, YH-Chem1, YH18421, DA-1241).

Item 169. Combination of a compound of formula I of any one of items 1 to 133 with GPR40 agonists (e.g. TUG-424, P-1736, P-11187, JTT-851, GW9508, CNX-011-67, AM-1638, AM-5262).

Item 170. Combination of a compound of formula I of any one of items 1 to 133 with GPR120 agonists and GPR142 agonists.

Item 171. Combination of a compound of formula I of any one of items 1 to 133 with Systemic or low-absorbable TGR5 (GPBAR1=G-protein-coupled bile acid receptor 1) agonists (e.g. INT-777, XL-475, SB756050).

Item 172. Combination of a compound of formula I of any one of items 1 to 133 with Diabetes immunotherapeutics, for example: oral C—C chemokine receptor type 2 (CCR-2) antagonists (e.g. CCX-140, JNJ-41443532), interleukin 1 beta (IL-1BR) antagonists (e.g. AC-201), or oral monoclonal antibodies (MoA) (e.g. methalozamide, VVP808, PAZ-320, P-1736, PF-05175157, PF-04937319).

Item 173. Combination of a compound of formula I of any one of items 1 to 133 with Anti-inflammatory agents for the treatment of the metabolic syndrome and diabetes, for example: nuclear factor kappa B inhibitors (e.g. Triolex®).

Item 174. Combination of a compound of formula I of any one of items 1 to 133 with Adenosine monophosphate-activated protein kinase (AMPK) stimulants, for example: Imeglimin (PXL-008), Debio-0930 (MT-63-78), R-118.

Item 175. Combination of a compound of formula I of any one of items 1 to 133 with Inhibitors of 11-beta-hydroxysteroid dehydrogenase 1 (11-beta-HSD-1) (e.g. LY2523199, BMS770767, RG-4929, BMS816336, AZD-8329, HSD-016, BI-135585).

Item 176. Combination of a compound of formula I of any one of items 1 to 133 with Activators of glucokinase (e.g. PF-04991532, TTP-399 (GK1-399), GKM-001 (ADV-1002401), ARRY-403 (AMG-151), TAK-329, TMG-123, ZYGK1).

Item 177. Combination of a compound of formula I of any one of items 1 to 133 with Inhibitors of diacylglycerol O-acyltransferase (DGAT) (e.g. pradigastat (LCQ-908)), inhibitors of protein tyrosine phosphatase 1 (e.g. trodusquemine), inhibitors of glucose-6-phosphatase, inhibitors of fructose-1,6-bisphosphatase, inhibitors of glycogen phosphorylase, inhibitors of phosphoenol pyruvate carboxykinase, inhibitors of glycogen synthase kinase, inhibitors of pyruvate dehydrogenase kinase.

Item 178. Combination of a compound of formula I of any one of items 1 to 133 with Modulators of glucose transporter-4, somatostatin receptor 3 agonists (e.g. MK-4256).

Item 179. Combination of a compound of formula I of any one of items 1 to 133 with one or more lipid lowering agents.

Item 180. Combination of a compound of formula I of any one of items 1 to 133 with 3-hydroxy-3-methylglutaryl-coenzym-A-reductase (HMG-CoA-reductase) inhibitors such as simvastatin (e.g. Zocor®, Inegy®, Simcor®), atorvastatin (e.g. Sortis®, Caduet®), rosuvastatin (e.g. Crestor®), pravastatin (e.g. Lipostat®, Selipran®), fluvastatin (e.g. Lescol®), pitavastatin (e.g. Livazo®, Livalo®), lovastatin (e.g. Mevacor®, Advicor®), mevastatin (e.g. Compactin®), rivastatin, cerivastatin (e.g. Lipobay®), fibrates such as bezafibrate (e.g. Cedur® retard), ciprofibrate (e.g. Hyperlipen®), fenofibrate (e.g. Antara®, Lipofen®, Lipanthyl®), gemfibrozil (e.g. Lopid®, Gevilon®), etofibrate, simfibrate, ronifibrate, clinofibrate, pemafibrate, clofibrate, clofibride, nicotinic acid and derivatives thereof (e.g. niacin, including slow release formulations of niacin), nicotinic acid receptor 1 agonists (e.g. GSK-256073), PPAR-delta agonists, acetyl-CoA-acetyltransferase (ACAT) inhibitors (e.g. avasimibe), cholesterol absorption inhibitors (e.g. ezetimibe, Ezetrol®, Zetia®, Liptruzet®, Vytorin®, S-556971), bile acid-binding substances (e.g. cholestyramine, colesevelam), ileal bile acid transport (IBAT) inhibitors (e.g. GSK-2330672, LUM-002), microsomal triglyceride transfer protein (MTP) inhibitors (e.g. Iomitapide (AEGR-733), SLx-4090, granotapide), modulators of proprotein convertase subtilisin/kexin type 9 (PCSK9) (e.g. alirocumab (e.g. Praluent®), evolocumab (e.g. Repatha®), LGT-209, PF-04950615, MPSK3169A, LY3015014, ALD-306, ALN-PCS, BMS-962476, SPC5001, ISIS-394814, 1B20, LGT-210, 1D05, BMS-PCSK9Rx-2, SX-PCK9, RG7652), LDL receptor up-regulators, for example liver selective thyroid hormone receptor beta agonists (e.g. eprotirome (KB-2115), MB07811, sobetirome (QRX-431), VIA-3196, ZYT1), HDL-raising compounds such as: cholesteryl ester transfer protein (CETP) inhibitors (e.g. anacetrapib (MK0859), dalcetrapib, evacetrapib, JTT-302, DRL-17822, TA-8995, R-1658, LY-2484595, DS-1442), or dual CETP/PCSK9 inhibitors (e.g. K-312), ATP-binding cassette (ABC1) regulators, lipid metabolism modulators (e.g. BMS-823778, TAP-301, DRL-21994, DRL-21995), phospholipase A2 (PLA2) inhibitors (e.g. darapladib, Tyrisa®, varespladib, rilapladib), ApoA-I enhancers (e.g. RVX-208, CER-001, MDCO-216, CSL-112), cholesterol synthesis inhibitors (e.g. ETC-1002), lipid metabolism modulators (e.g. BMS-823778, TAP-301, DRL-21994, DRL-21995) and omega-3 fatty acids and derivatives thereof (e.g. icosapent ethyl (AMR101), Epanova®, Lovaza®, Vascepa®, AKR-063, NKPL-66, PRC-4016, CAT-2003).

Item 181. Combination of a compound of formula I of any one of items 1 to 133 with HDL-raising compounds such as: CETP inhibitors (e.g. Torcetrapib, Anacetrapid, Dalcetrapid, Evacetrapid, JTT-302, DRL-17822, TA-8995) or ABC1 regulators.

Item 182. Combination of a compound of formula I of any one of items 1 to 133 with one or more active substances for the treatment of obesity.

Item 183. Combination of a compound of formula I of any one of items 1 to 133 with Bromocriptine (e.g. Cycloset®, Parlodel®), phentermine and phentermine formulations or combinations (e.g. Adipex-P, lonamin, Qsymia®), benzphetamine (e.g. Didrex®), diethylpropion (e.g. Tenuate®), phendimetrazin (e.g. Adipost®, Bontril®), bupropion and combinations (e.g. Zyban®, Wellbutrin XL®, Contrave®, Empatic®), sibutramine (e.g. Reductil®, Meridia®), topiramat (e.g. Topamax®), zonisamid (e.g. Zonegran®), tesofensine, opioid antagonists such as naltrexone (e.g. Naltrexin®, naltrexone and bupropion), cannabinoid receptor 1 (CB1) antagonists (e.g. TM-38837), melanin-concentrating hormone (MCH-1) antagonists (e.g. BMS-830216, ALB-127158(a)), MC4 receptor agonists and partial agonists (e.g. AZD-2820, RM-493), neuropeptide Y5 (NPY5) or NPY2 antagonists (e.g. velneperit, S-234462), NPY4 agonists (e.g. PP-1420), beta-3-adrenergic receptor agonists, leptin or leptin mimetics, agonists of the 5-hydroxytryptamine 2c (5HT2c) receptor (e.g. lorcaserin, Belviq®), pramlintide/metreleptin, lipase inhibitors such as cetilistat (e.g. Cametor®), orlistat (e.g. Xenical®, Calobalin®), angiogenesis inhibitors (e.g. ALS-L1023), betahistidin and histamine H3 antagonists (e.g. HPP-404), AgRP (agouti related protein) inhibitors (e.g. TTP-435), serotonin re-uptake inhibitors such as fluoxetine (e.g. Fluctine®), duloxetine (e.g. Cymbalta®), dual or triple monoamine uptake inhibitors (dopamine, norepinephrine and serotonin re-uptake) such as sertraline (e.g. Zoloft®), tesofensine, methionine aminopeptidase 2 (MetAP2) inhibitors (e.g. beloranib), and antisense oligonucleotides against production of fibroblast growth factor receptor 4 (FGFR4) (e.g. ISIS-FGFR4Rx) or prohibitin targeting peptide-1 (e.g. Adipotide®).

Item 184. Combination of a compound of formula I of any one of items 1 to 133 with one or more active substances for the treatment of fatty liver diseases including non-alcoholic fatty liver disease (NAFLD) and non-alcoholic steatohepatitis (NASH).

Item 185. Combination of a compound of formula I of any one of items 1 to 133 with Insulin sensitizers (e.g. rosiglitazone, pioglitazone), other PPAR modulators (e.g. elafibranor, saroglitazar, IVA-337), FXR agonists (e.g. obethicolic acid (INT-747), GS-9674, LJN-452, EDP-305), FGF19 analogues (e.g. NGM-282), FGF21 analogues (PF-05231023), GLP-1 analogues (e.g. Iiraglutide), SCD1 inhibitors (e.g. aramchol), anti-inflammatory compounds (e.g. CCR2/CCR5 antagonist cenicriviroc, pentamidine VLX-103), compounds reducing oxidative stress (e.g. ASK1 inhibitor GS-4997, VAP-1 inhibitor PXS-4728A), caspase inhibitors (e.g. emricasan), LOXL2 inhibitors (e.g. simtuzumab), galectin-3 protein inhibitors (e.g. GR-MD-02).

Item 186. Combination of a compound of formula I of any one of items 1 to 133 with with drugs for influencing high blood pressure, chronic heart failure or atherosclerosis.

Item 187. Combination of a compound of formula I of any one of items 1 to 133 with nitric oxide donors, AT1 antagonists or angiotensin II (AT2) receptor antagonists such as telmisartan (e.g. Kinzal®, Micardis®), candesartan (e.g. Atacand®, Blopress®), valsartan (e.g. Diovan®, Co-Diovan®), losartan (e.g. Cosaar®), eprosartan (e.g. Teveten®), irbesartan (e.g. Aprovel®, CoAprovel®), olmesartan (e.g. Votum®, Olmetec®), tasosartan, azilsartan (e.g. Edarbi®), dual angiotensin receptor blockers (dual ARBs), angiotensin converting enzyme (ACE) inhibitors, ACE-2 activators, renin inhibitors, prorenin inhibitors, endothelin converting enzyme (ECE) inhibitors, endothelin receptor (ET1/ETA) blockers, endothelin antagonists, diuretics, aldosterone antagonists, aldosterone synthase inhibitors, alpha-blockers, antagonists of the alpha-2 adrenergic receptor, beta-blockers, mixed alpha-/beta-blockers, calcium antagonists, calcium channel blockers (CCBs), nasal formulations of the calcium channel blocker diltiazem (e.g. CP-404), dual mineralocorticoid/CCBs, centrally acting antihypertensives, inhibitors of neutral endopeptidase, aminopeptidase-A inhibitors, vasopeptide inhibitors, dual vasopeptide inhibitors such as neprilysin-ACE inhibitors or neprilysin-ECE inhibitors, dual-acting AT receptor-neprilysin inhibitors, dual AT1/ETA antagonists, advanced glycation end-product (AGE) breakers, recombinant renalase, blood pressure vaccines such as anti-RAAS (renin-angiotensin-aldosteron-system) vaccines, AT1- or AT2-vaccines, drugs based on hypertension pharmacogenomics such as modulators of genetic polymorphisms with antihypertensive response, thrombocyte aggregation inhibitors.

Item 188. The use of the compounds of formula I of any one of items 1 to 133, or a physiologically acceptable salt thereof, in combination with one or more active substances according to any one of items 140 to 187 may take place simultaneously, separately or sequentially.

Item 189. The use of the compounds of formula I of any one of items 1 to 139, or a physiologically acceptable salt thereof in combination with another active substance according to any one of items 140 to 187 may take place simultaneously or at staggered times.

TABLE 19
Sequences

| SEQ | |
|---|---|
| ID | |
| NO | sequence |
| 1 | H2N-Y-A-E-G-T-F-I-S-D-Y-S-I-A-M-D-K-I-H-Q-Q-D-F-V-N-W-I-L-A-Q- |
| | K-G-K-K-N-D-W-K-H-N-I-T-Q-OH |
| 2 | H2N-H-A-E-G-T-F-T-S-D-V-S-S-Y-I-E-G-Q-A-A-K-E-F-I-A-W-I-V-K-G- |
| | R-NH2 |
| 3 | H2N-H-G-E-G-T-F-T-S-D-I-S-K-Q-M-E-E-E-A-V-R-I-F-I-E-W-I-K-N-G- |
| | G-P-S-S-G-A-P-P-P-S-NH2 |
| 4 | H2N-Y-Aib-E-G-T-F-I-S-D-I-S-I-Aib-K[{AEEA}2-gGlu-C18OH]-D-R-I-H- |
| | Q-Aib-E-F-I-E-W-I-L-A-Q-G-P-S-S-G-A-P-P-P-S-NH2 |
| 5 | H2N-Y-Aib-E-G-T-F-I-S-D-I-S-I-Aib-K[{Gly}3-gGlu-C18OH]-D-R-I-H-Q- |
| | Aib-E-F-I-E-W-I-L-A-Q-G-P-S-S-G-A-P-P-P-S-NH2 |
| 6 | H2N-Y-Aib-E-G-T-F-I-S-D-I-S-I-Aib-K[{N-MeGly}3-gGlu-C18OH]-D-R-I- |
| | H-Q-Aib-E-F-I-E-W-I-L-A-Q-G-P-S-S-G-A-P-P-P-S-NH2 |
| 7 | H2N-Y-Aib-E-G-T-F-I-S-D-I-S-I-Aib-K[{AEEA}2-gGlu-C20OH]-D-R-I-H- |
| | Q-Aib-E-F-I-E-W-I-L-A-Q-G-P-S-S-G-A-P-P-P-S-NH2 |
| 8 | MeHN-Y-Aib-E-G-T-F-I-S-D-I-S-I-Aib-K[{AEEA}2-gGlu-C180H]-D-R-I- |
| | H-Q-Aib-E-F--E-W-I-L-A-Q-G-P-S-S-G-A-P-P-P-S-NH2 |
| 9 | H2N-Y-Aib-E-G-T-F-I-S-D-I-S-I-Aib-K[{AEEA}2-gGlu-C18OH]-D-R-I-H- |
| | Q-E-E-F-I-E-W-I-L-A-Q-G-P-S-S-G-A-P-P-P-S-NH2 |
| 10 | H2N-Y-Aib-E-G-T-F-I-S-D-I-S-I-Aib-K[{AEEA}2-gGlu-C20OH]-D-R-I-H- |
| | Q-E-E-F-I-E-W-I-L-A-Q-G-P-S-S-G-A-P-P-P-S-NH2 |
| 11 | H2N-Y-Aib-E-G-T-F-I-S-D-I-S-I-Aib-K[{AEEA}2-gGlu-C18OH]-D-R-I-H- |
| | Q-E-E-F-I-E-W-I-L-A-Q-G-P-S-S-G-A-P-P-P-S-OH |
| 12 | H2N-Y-Aib-E-G-T-F-I-S-D-I-S-I-Aib-K[{AEEA}3-gGlu-C18OH]-D-R-I-H- |
| | Q-E-E-F-I-E-W-I-L-A-Q-G-P-S-S-G-A-P-P-P-S-NH2 |
| 13 | MeHN-Y-Aib-E-G-T-F-I-S-D-I-S-I-Aib-K[{AEEA}2-gGlu-C18OH]-D-R-I- |
| | H-Q-E-E-F-I-E-W-I-L-A-Q-G-P-S-S-G-A-P-P-P-S-NH2 |
| 14 | H2N-Y-Aib-E-G-T-F-I-S-D-I-S-I-Aib-K[{AEEA}2-{gGlu}2-C18OH]-D-R-I- |
| | H-Q-E-E-F-I-E-W-I-L-A-Q-G-P-S-S-G-A-P-P-P-S-NH2 |
| 15 | H2N-Y-Aib-E-G-T-F-I-S-D-I-S-I-Aib-K[{AEEA}2-{gGlu}2-C20OH]-D-R-I- |
| | H-Q-E-E-F-I-E-W-I-L-A-Q-G-P-S-S-G-A-P-P-P-S-NH2 |
| 16 | H2N-Y-Aib-E-G-T-F-I-S-D-I-S-I-Aib-K[{Gly}3-gGlu-C18OH]-D-R-I-H-Q- |
| | E-E-F-I-E-W-I-L-A-Q-G-P-S-S-G-A-P-P-P-S-NH2 |
| 17 | H2N-Y-Aib-E-G-T-F-I-S-D-I-S-I-Aib-K[{N-MeGly}3-gGlu-C18OH]-D-R-I- |
| | H-Q-E-E-F-I-E-W-I-L-A-Q-G-P-S-S-G-A-P-P-P-S-NH2 |
| 18 | H2N-Y-Aib-E-G-T-F-I-S-D-I-S-I-Aib-K[{AEEA}2-{gGlu}2-C18OH]-D-R-I- |
| | H-Q-Aib-E-F-I-E-W-I-L-A-Q-G-P-S-S-G-A-P-P-P-S-NH2 |
| 19 | H2N-Y-Aib-E-G-T-F-I-S-D-I-S-I-Aib-K[{AEEA}2-{gGlu}2-C20OH]-D-R-I- |
| | H-Q-Aib-E-F-I-E-W-I-L-A-Q-G-P-S-S-G-A-P-P-P-S-NH2 |
| 20 | H2N-Y-Aib-E-G-T-F-I-S-D-I-S-I-Aib-K[{AEEA}2-gGlu-C18OH]-D-R-I-H- |
| | Q-Aib-E-F-I-E-W-I-L-A-Q-G-P-S-S-G-A-P-P-P-S-OH |

1. A compound of formula I:
I
R¹HN-Tyr-Aib-Glu-Gly-Thr-Phe-Ile-Ser-Asp-Leu-
Ser-Ile-Aib-X14-Asp-Arg-Ile-His[[ ]_Gln-X20-
Glu-Phe-Ile-Glu-Trp-Leu-Leu-Ala-GIn-Gly-Pro-
Ser-Ser-Gly-Ala-Pro-Pro-Pro-Ser-R²
wherein
R¹ is H or C₁-C₄-alkyl;
X14 represents lysine (Lys), wherein the —NH₂ side chain group is functionalized by-Z1-Z2-C(O)—R⁵, wherein
—Z1-Z2 represents a linker in all stereoisomeric forms comprising 1 to 5 amino acid linker groups selected from the group consisting of gamma-glutamate (gGlu), glycine (Gly), N-Methyl-glycine (N-MeGly), and 8-amino-3,6-dioxa-octanoic acid (AEEA), and
R⁵ is an acyclic linear or branched (C₈-C₃₀) saturated or unsaturated hydrocarbon group, which is unsubstituted or substituted by halogen, —OH, and/or —CO₂H;
X20 represents Glu or 2-aminoisobutyric acid (Aib);
R² is NH₂ or OH;
or a salt or solvate thereof.

2. The compound of claim 1,
wherein
Z1 represents a group selected from {AEEA}2, {AEEA}3, {Gly}3, and {N-MeGly}3;
Z2 represents gGlu or gGlu-gGlu; and
R⁵ represents —(CH2)x-COOH, wherein x is an integer from 15 to 22.

3. The compound of claim 1,
wherein the —Z1-Z2-C(O)—R⁵ group is selected from any of:
[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl-,
[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]a cetyl-,
[2-[2-[2-[[2-[2-[2-[[2-[2-[2-[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl-,
[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]-ethoxy]acetyl]amino]ethoxy]ethoxy]acetyl-,
[2-[2-[2-[[2-[2-[2-[[(4S)-4-carboxy-4-[[(4S)-4-carboxy-4-(19-carboxynonadecanoylamino)butanoyl]amino]butanoyl]amino]ethoxy]-ethoxy]acetyl]amino]ethoxy]ethoxy]acetyl-,
[2-[[2-[[2-[[(4S)-4-carboxy-4-(17-carboxyheptadecanoyl-amino)butanoyl]amino]acetyl]amino]acetyl]amino]acetyl]-, and
[2-[methyl-[2-[methyl-[2-[methyl-[(4S)-4-carboxy-4-(17-carboxy-heptadecanoylamino)butanoyl]amino]acetyl]amino]acetyl]amino]acetyl].

4. The compound of claim 1, wherein
R¹ is H or methyl, and
X20 is Glu.

5. The compound of claim 1,
wherein
R¹ is H or methyl, and
X20 is Aib.

6. The compound of claim 1,
wherein
R¹ is H, and
R² is NH₂.

7. The compound of claim 1,
wherein
R¹ is H,
X20 is Glu, and
R² is NH₂.

8. The compound of claim 1, wherein
R² represents NH₂.

9. The compound of claim 1, wherein
R² represents OH.

10. The compound of claim 1, wherein the compound comprises a sequence of any of SEQ ID NOs: 4-20, or a salt or solvate thereof.

11. The compound of claim 1, wherein the compound comprises the sequence of SEQ ID NO: 9, or a salt or solvate thereof.

12. (canceled)

13. A pharmaceutical composition comprising the compound of claim 1 and a pharmaceutically acceptable carrier.

14. A method of treating a disease in an individual, comprising administering to the individual an effective amount of the compound of claim 1, wherein the disease is selected from the group consisting of glucose intolerance, insulin resistance, pre-diabetes, increased fasting glucose, hyperglycemia, type 2 diabetes or complications thereof, impaired glucose tolerance, type 1 diabetes or complications thereof, hypertension, dyslipidemia, metabolic syndrome, obesity or complications thereof, atherosclerosis, arteriosclerosis, coronary heart disease, peripheral artery disease, stroke, non-alcoholic fatty liver disease (NAFLD), non-alcoholic steatohepatitis (NASH), osteoporosis, nausea, vomiting, and any combination thereof.

15. The method of claim 14, wherein the method achieves one or more of: control of appetite, control of feeding and caloric intake, prevention of weight gain, promotion of weight loss, and reduction of excess body weight.

16-18. (canceled)

19. The method of claim 14, wherein the method achieves one or more of: reducing blood glucose levels, and reducing HbA1c levels.

20-21. (canceled)

22. The compound of claim 1, which is an agonist of gastric inhibitory polypeptide (GIP) receptor.

23. The compound of claim 1, which has one or more properties selected from:
(i) having a lower EC50 for GIP receptor than for glucagon-like peptide 1 (GLP-1) receptor;
(ii) having a high solubility at pH 6 to 8;
(iii) having a high solubility at physiological pH in the presence of an antimicrobial preservative;
(iv) having a high chemical stability when stored in solution;
(v) having a high physical stability;
(vi) having an improved pharmacokinetic property; and
(vii) improving glucose tolerance in vivo.

24. The method of claim 14, comprising further administering to the individual an effective amount of one or more of: an antidiabetic agent, an GLP-1 receptor agonist, an GLP-2 receptor agonist, an GIP receptor agonist, and a glucagon receptor agonist.

25. The method of claim 14, wherein the compound is administered intravenously or subcutaneously.
